(19) United States
(12) Patent Application Publication
(10) Pub. No.: US 2015/0155498 A1
(43) Pub. Date: Jun. 4, 2015

(54) NOVEL ORGANIC ELECTROLUMINESCENCE COMPOUNDS AND ORGANIC ELECTROLUMINESCENCE DEVICE CONTAINING THE SAME

(71) Applicant: Rohm and Haas Electronic Materials Korea Ltd., Cheonan (KR)

(72) Inventors: Hee-Choon Ahn, Suwon (KR); Bong-Ok Kim, Seoul (KR); Seung-Ae Kim, Anyang (KR); Yong-Gil Kim, Suwon (KR); Kyung-Joo Lee, Seoul (KR); Tae-Jin Lee, Seoul (KR); Hyuck-Joo Kwon, Seoul (KR)

(21) Appl. No.: 14/400,415

(22) PCT Filed: May 30, 2013
(86) PCT No.: PCT/KR2013/004742

§ 371 Date: Nov. 11, 2014

(30) Foreign Application Priority Data
May 30, 2012 (KR) 10-2012-0057553

(51) Int. Cl. H01L 51/00; C07D 495/04

(52) U.S. Cl. CPC H01L 51/0061; C07D 495/04; H01L 51/0071; H01L 51/0072; H01L 51/5012

(57) ABSTRACT

The present invention relates to a novel organic electroluminescent compound and an organic electroluminescent device comprising the same. Using the organic electroluminescent compound according to the present invention, it is possible to manufacture an OLED device of lowered driving voltages and advanced power efficiency.

### TECHNICAL FIELD

The present invention relates to novel organic electroluminescence compounds and organic electroluminescence device containing the same.

### BACKGROUND ART

An electroluminescent (EL) device is a self-light-emitting device which has advantages in that it provides a wider viewing angle, a greater contrast ratio, and a faster response time. An organic EL device was first developed by Eastman Kodak, by using small aromatic diamine molecules, and aluminum complexes as materials for forming a light-emitting layer [Appl. Phys. Lett. 51, 913, 1987].

The most important factor determining luminous efficiency in an organic EL device is the light-emitting material. Until now, fluorescent materials have been widely used as a light-emitting material. However, in view of electroluminescent mechanisms, since phosphorescent materials theoretically enhance luminous efficiency by four (4) times compared to fluorescent materials, development of phosphorescent light-emitting materials are widely being researched. Iridium(III) complexes have been widely known as phosphorescent materials, including bis(2-(2′-benzothienyl)-pyridinato-N,C3′)iridium(acetylacetonate) ((acac)Ir(btp)₂), tris(2-phenylpyridine)iridium (Ir(ppy)₃) and bis(4,6-difluorophenylpyridinato-N,C2)picolinate iridium (Firpic) as red, green and blue materials, respectively.

A luminescent material (dopant) can be used in combination with a host material as a light emitting material to improve color purity, luminous efficiency, and stability. Since host materials greatly influence the efficiency and performance of the EL device when using a host material/dopant system as a light emitting material, their selection is important.

At present, 4,4′—N,N′-dicarbazol-biphenyl (CBP) is the most widely known host material for phosphorescent substances. Recently, Pioneer (Japan) et al. developed a high performance organic EL device using bathocuproine (BCP) and aluminum(III)bis(2-methyl-8-quinolinate)(4-phenylphenolate) (BAIq) etc. as host materials, which were known as hole blocking layer materials.

Though these phosphorous host materials provide good light-emitting characteristics, they have the following disadvantages: (1) Due to their low glass transition temperature and poor thermal stability, their degradation may occur during a high-temperature deposition process in a vacuum. (2) The power efficiency of an organic EL device is given by [(π/voltage)×current efficiency], and the power efficiency is inversely proportional to the voltage. Although an organic EL device comprising phosphorescent host materials provides higher current efficiency (cd/A) than one comprising fluorescent materials, a significantly high driving voltage is necessary. Thus, there is no merit in terms of power efficiency (lm/W). (3) Further, the operational lifespan of an organic EL device is short and luminous efficiency is still required to be improved.

Meanwhile, copper phthalocyanine (CuPc), 4,4′-bis[N-(1-naphthyl)-N-phenylamino]biphenyl (NPB), N,N′-diphenyl-N,N′-bis(3-methylphenyl)-(1,1′-biphenyl)-4,4′-diamine (TPD), 4,4′,4″-tris(3-methylphenylphenylamino)triphenylamine (MTDATA), etc. were used as a hole injection and transport material.

However, an organic EL device using these materials is problematic in quantum efficiency and operational lifespan. It is because, when an organic EL device is driven under high current, thermal stress occurs between an anode and the hole injection layer. Thermal stress significantly reduces the operational lifespan of the device. Further, since the organic material used in the hole injection layer has very high hole mobility, the hole-electron charge balance may be broken and quantum yield (cd/A) may decrease.

International Patent Publication No. WO 2009/148015 discloses a compound for an organic EL device in which a heteroaryl such as carbazole, dibenzothiophene, or dibenzofuran is bonded at the carbon atom position of a structure of a polycyclic compound formed by fluorene, carbazole, dibenzofuran, or dibenzothiophene fused with an indene, indole, benzofuran, or benzothiophene.

In addition, US Patent Appln. Laying-Open No. 2011/0279020 A1 discloses a compound for an organic electroluminescent device in which two carbazole moieties are bonded via a carbon-carbon single bond.

However, the above references do not specifically disclose a compound in which an arylamine or a heteroarylamine is bonded at the nitrogen atom position of a structure of a polycyclic compound formed by carbazole fused with an indene, indole, benzofuran, or benzothiophene.

### DISCLOSURE OF THE INVENTION

### Problems to be Solved

The objective of the present invention is to provide an organic electroluminescent compound which has higher luminous efficiency and a longer operational lifespan than the conventional materials; and an organic electroluminescent device having high efficiency and a long lifespan, using said compounds.

### Solution to Problems

The present inventors found that the above objective can be achieved by a compound represented by the following formula 1:

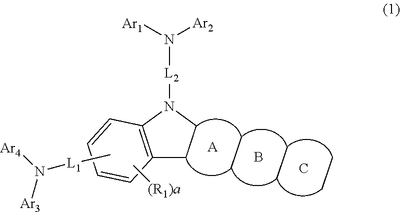

wherein

ring A represents an aromatic ring of

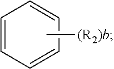

ring B represents a pentacyclic ring of

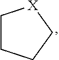

where X represents —O—, —S—, —N(R₄)—, —C(R₅)(R₆)— or —Si(R₇)(R₈)—;

ring C represents an aromatic ring of

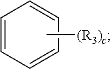

L₁ represents a single bond, a substituted or unsubstituted 5- to 30-membered heteroarylene, or a substituted or unsubstituted (C6-C30)arylene;

L₂ represents a substituted or unsubstituted 5- to 30-membered heteroarylene, or a substituted or unsubstituted (C6-C30)arylene;

Ar₁ to Ar₄ each independently represent a substituted or unsubstituted 5- to 30-membered heteroaryl, or a substituted or unsubstituted (C6-C30)aryl;

R₁ to R₃ each independently represent hydrogen, deuterium, a halogen, a substituted or unsubstituted (C1-C30)alkyl, a substituted or unsubstituted (C6-C30)aryl, a substituted or unsubstituted 5- to 30-membered heteroaryl, —NR₁₁R₁₂ or —SiR₁₃R₁₄R₁₅; or are linked to an adjacent substituent(s) to form a mono- or polycyclic, 3- to 30-membered alicyclic or aromatic ring whose carbon atom(s) may be replaced with at least one hetero atom selected from the group consisting of nitrogen, oxygen and sulfur;

R₄ to R₈, and R₁₁ to R₁₅ each independently represent hydrogen, deuterium, a halogen, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic, 3- to 30-membered alicyclic or aromatic ring;

a represents an integer of 1 to 3; where a is an integer of 2 or more, each of the substituent may be same or different;

b represents 1 or 2; where b is 2, each of the substituent may be same or different;

c represents an integer of 1 to 4; where c is an integer of 2 or more, each of the substituent may be same or different; and

the heteroaryl(ene) contains at least one hetero atom selected from B, N, O, S, P(═O), Si and P.

### Effects of the Invention

The organic electroluminescent compound according to the present invention can manufacture an organic electroluminescent device which has high luminous efficiency and a long operational lifespan. In addition, using the compound according to the present invention, it is possible to manufacture an electroluminescent device of lowered driving voltages and advanced power efficiency.

### EMBODIMENTS OF THE INVENTION

Hereinafter, the present invention will be described in detail. However, the following description is intended to explain the invention, and is not meant in any way to restrict the scope of the invention.

Hereinafter, the compound represented by the above formula 1 will be described in detail.

The compound represented by formula 1 has a structure characterized as follows:

(1) A carbazole structure is fused with an aryl or a heteroaryl, such as indene, indole, benzofuran, and benzothiophene, (2) an arylamine or a heteroarylamine is bonded to the carbazole structure at the nitrogen atom position via a linker, and (3) an arylamine or a heteroarylamine is bonded to the carbazole structure at the carbon atom position, directly or via a linker,

The compound represented by formula 1 is preferably represented by one selected from formulae 2 to 4:

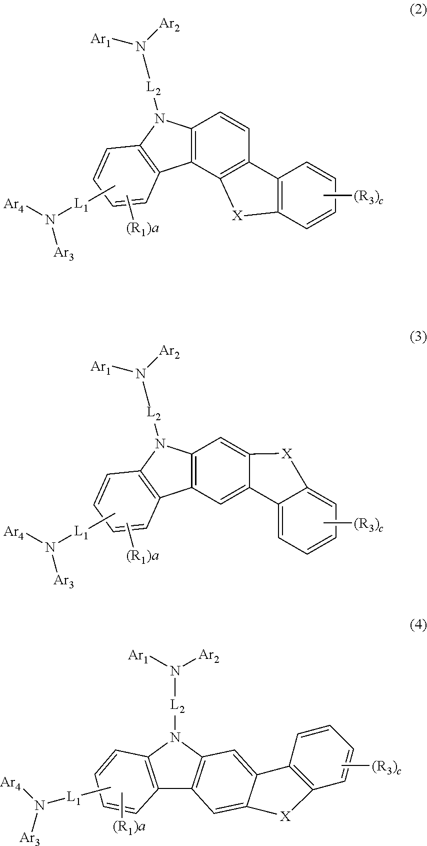

wherein X, L₁, L₂, Ar₁ to Ar₄, R₁, R₃, a and c are as defined in formula 1.

In formula 1 to 4 above, X preferably represents —O—, —S—, —N(R₄)— or —C(R₅)(R₆)—, where R₄ preferably represents a substituted or unsubstituted (C6-C15)aryl, more preferably represents an unsubstituted (C6-C15)aryl; and R₅ and R₆ preferably each independently represent a substituted or unsubstituted (C1-C10)alkyl, or a substituted or unsubstituted (C6-C15)aryl, more preferably each independently represent an unsubstituted (C1-C10)alkyl, or an unsubstituted (C6-C15)aryl.

In formula 1 to 4 above, L₁ preferably represents a single bond, a substituted or unsubstituted (C6-C15)arylene, more preferably represents a single bond, an unsubstituted (C6-C15)arylene, or a (C6-C15)arylene substituted with a (C1-C6)alkyl or a (C6-C12)aryl.

In formula 1 to 4 above, L₂ preferably represents a substituted or unsubstituted (C6-C15)arylene, more preferably represents an unsubstituted (C6-C15)arylene, or a (C6-C15)arylene substituted with a (C1-C6)alkyl or a (C6-C12)aryl.

In formula 1 to 4 above, Ar₁ to Ar₄ preferably each independently represent a substituted or unsubstituted 5- to 15-membered heteroaryl, or a substituted or unsubstituted (C6-C20)aryl, more preferably each independently represent an unsubstituted 5- to 15-membered heteroaryl, a 5- to 15-membered heteroaryl substituted with a (C6-C15)aryl, an unsubstituted (C6-C20)aryl, or a (C6-C20)aryl substituted with a (C1-C6)alkyl or a (C6-C15)aryl.

In formula 1 to 4 above, R₁ to R₃ preferably each independently represent hydrogen, a substituted or unsubstituted (C1-C10)alkyl, a substituted or unsubstituted (C6-C15)aryl, a substituted or unsubstituted 5- to 15-membered heteroaryl, —NR₁₁R₁₂ or —SiR₁₃R₁₄R₁₅, more preferably each independently represent hydrogen, an unsubstituted (C1-C10)alkyl, an unsubstituted (C6-C15)aryl, a (C6-C15)aryl substituted with a (C6-C15)aryl, an unsubstituted 5- to 15-membered heteroaryl, a 5- to 15-membered heteroaryl substituted with a (C1-C6)alkyl, —NR₁₁R₁₂ or —SiR₁₃R₁₄R₁₅. Herein, R₁₁ and R₁₂ preferably each independently represent a substituted or unsubstituted (C6-C15)aryl, more preferably each independently represent an unsubstituted (C6-C15)aryl; and R₁₃ to R₁₅ preferably each independently represent a substituted or unsubstituted (C1-C10)alkyl, more preferably each independently represent an unsubstituted (C1-C10)alkyl.

Herein, “(C1-C30)alkyl” is meant to be a linear or branched alkyl having 1 to 30 carbon atoms, in which the number of carbon atoms is preferably 1 to 20, more preferably 1 to 10, and includes methyl, ethyl, n-propyl, isopropyl, n-butyl, isobutyl, tert-butyl, etc.; “(C2-C30) alkenyl” is meant to be a linear or branched alkenyl having 2 to 30 carbon atoms, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc.; “(C2-C30)alkynyl” is a linear or branched alkynyl having 2 to 30 carbon atoms, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc.; “(C3-C30)cycloalkyl” is a mono- or polycyclic hydrocarbon having 3 to 30 carbon atoms, in which the number of carbon atoms is preferably 3 to 20, more preferably 3 to 7, and includes cyclopropyl, cyclobutyl, cyclopentyl, cyclohexyl, etc.; “3- to 7-membered heterocycloalkyl” is a cycloalkyl having at least one heteroatom selected from B, N, O, S, P(═O), Si and P, preferably O, S and N, and 3 to 7 ring backbone atoms, and includes tetrahydrofuran, pyrrolidine, thiolan, tetrahydropyran, etc.; “(C6-C30)aryl(ene)” is a monocyclic or fused ring derived from an aromatic hydrocarbon having 6 to 30 carbon atoms, in which the number of carbon atoms is preferably 6 to 20, more preferably 6 to 12, and includes phenyl, biphenyl, terphenyl, naphthyl, fluorenyl, phenanthrenyl, anthracenyl, indenyl, triphenylenyl, pyrenyl, tetracenyl, perylenyl, chrysenyl, naphthacenyl, fluoranthenyl, etc.; “5- to 30-membered heteroaryl(ene)” is an aryl group having at least one, preferably 1 to 4 heteroatom selected from the group consisting of B, N, O, S, P(═O), Si and P, and 5 to 30 ring backbone atoms; is a monocyclic ring, or a fused ring condensed with at least one benzene ring; has preferably 5 to 21, more preferably 5 to 15 ring backbone atoms; may be partially saturated; may be one formed by linking at least one heteroaryl or aryl group to a heteroaryl group via a single bond(s); and includes a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. Further, “halogen” includes F, Cl, Br and I.

Herein, “substituted” in the expression “substituted or unsubstituted” means that a hydrogen atom in a certain functional group is replaced with another atom or group, i.e., a substituent.

The substituents of the substituted alkyl, the substituted aryl(ene), and the substituted heteroaryl(ene) in L₁, L₂, Ar₁ to Ar₄, R₁ to R₈, and R₁₁ to R₁₅ in the above formulae 1 to 4 each independently are at least one selected from the group consisting of deuterium, a halogen, a cyano, a carboxyl, a nitro, a hydroxyl, a (C1-C30)alkyl, a halo(C1-C30)alkyl, a (C6-C30)aryl, a 5- to 30-membered heteroaryl, a 5- to 30-membered heteroaryl substituted with a (C6-C30)aryl, a (C6-C30)aryl substituted with a 5- to 30-membered heteroaryl, a (C3-C30)cycloalkyl, a 3- to 7-membered heterocycloalkyl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, a (C2-C30)alkenyl, a (C2-C30)alkynyl, a mono- or di-(C1-C30)alkylamino, a mono- or di-(C6-C30)arylamino, a (C1-C30)alkyl(C6-C30)arylamino, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl, and preferably each independently are at least one selected from the group consisting of a (C1-C10)alkyl or a (C6-C15)aryl.

The representative compounds of the present invention include the following compounds:

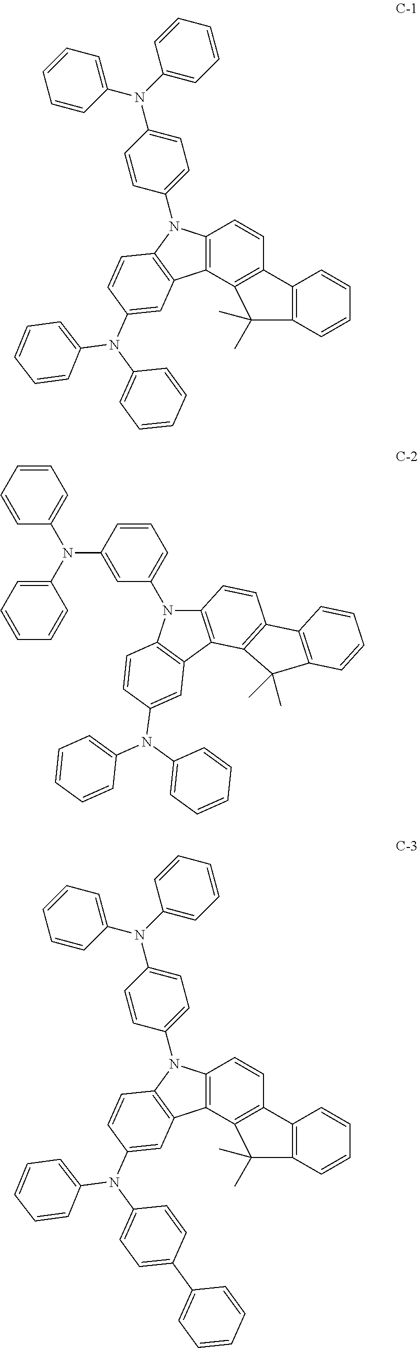

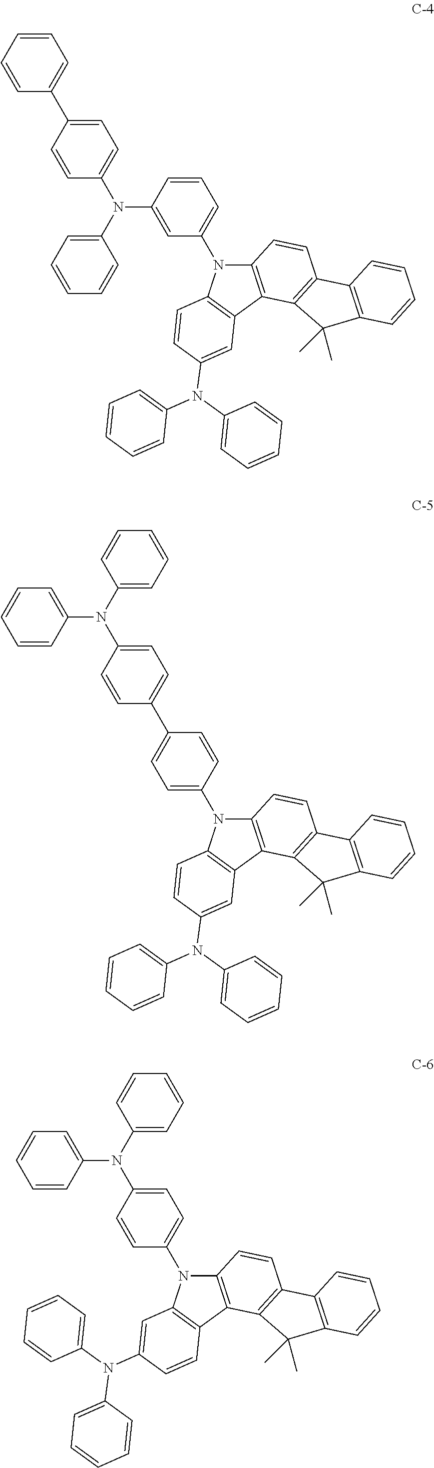 [SMILES: CC1(C)c2ccccc2-c2ccc3c(c21)c1cc(N(c2ccccc2)c2ccccc2)ccc1n3-c1cccc(N(c2ccccc2)c2ccc(-c3ccccc3)cc2)c1 ; CC1(C)c2ccccc2-c2ccc3c(c21)c1cc(N(c2ccccc2)c2ccccc2)ccc1n3-c1ccc(-c2ccc(N(c3ccccc3)c3ccccc3)cc2)cc1 ; CC1(C)c2ccccc2-c2ccc3c(c21)c1ccc(N(c2ccccc2)c2ccccc2)cc1n3-c1ccc(N(c2ccccc2)c2ccccc2)cc1]

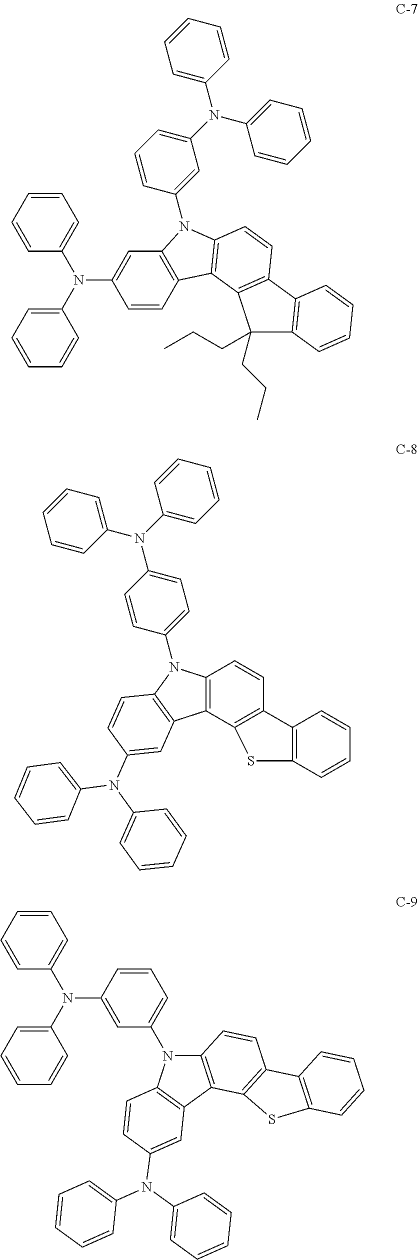 [SMILES: CCCC1(CCC)c2ccccc2-c2ccc3c(c21)c1ccc(N(c2ccccc2)c2ccccc2)cc1n3-c1cccc(N(c2ccccc2)c2ccccc2)c1 ; c1ccc(N(c2ccccc2)c2ccc(-n3c4ccc(N(c5ccccc5)c5ccccc5)cc4c4c5sc6ccccc6c5ccc43)cc2)cc1 ; c1ccc(N(c2ccccc2)c2cccc(-n3c4ccc(N(c5ccccc5)c5ccccc5)cc4c4c5sc6ccccc6c5ccc43)c2)cc1]

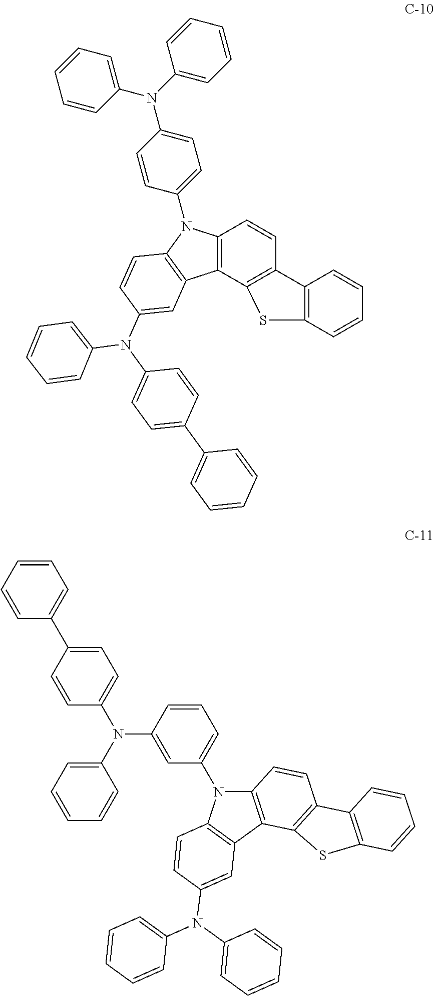 [SMILES: c1ccc(-c2ccc(N(c3ccccc3)c3ccc4c(c3)c3c5sc6ccccc6c5ccc3n4-c3ccc(N(c4ccccc4)c4ccccc4)cc3)cc2)cc1 ; c1ccc(-c2ccc(N(c3ccccc3)c3cccc(-n4c5ccc(N(c6ccccc6)c6ccccc6)cc5c5c6sc7ccccc7c6ccc54)c3)cc2)cc1]

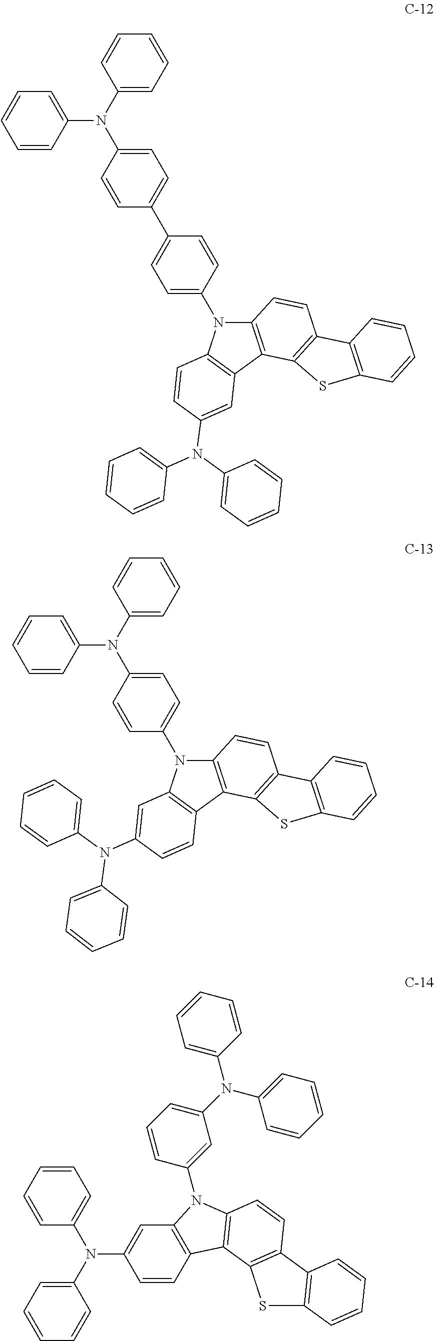

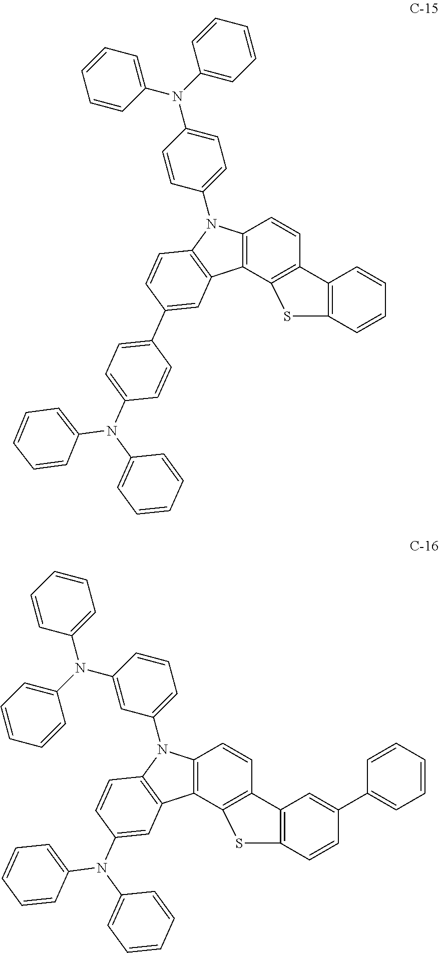 [SMILES: c1ccc(N(c2ccccc2)c2ccc(-c3ccc4c(c3)c3c5sc6ccccc6c5ccc3n4-c3ccc(N(c4ccccc4)c4ccccc4)cc3)cc2)cc1 ; c1ccc(-c2ccc3sc4c(ccc5c4c4cc(N(c6ccccc6)c6ccccc6)ccc4n5-c4cccc(N(c5ccccc5)c5ccccc5)c4)c3c2)cc1]

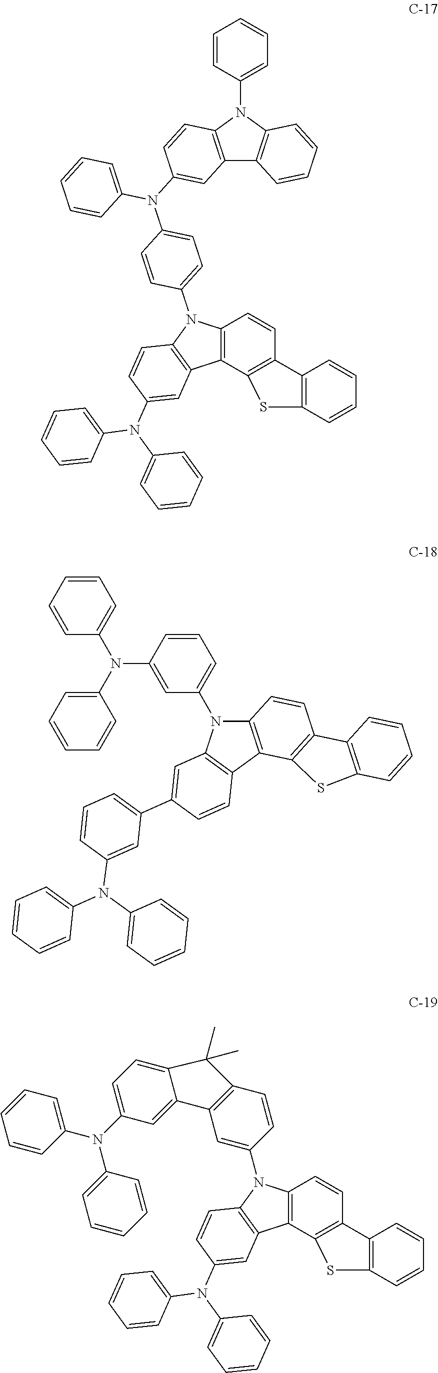 [SMILES: c1ccc(N(c2ccc(-n3c4ccc(N(c5ccccc5)c5ccccc5)cc4c4c5sc6ccccc6c5ccc43)cc2)c2ccc3c(c2)c2ccccc2n3-c2ccccc2)cc1 ; c1ccc(N(c2ccccc2)c2cccc(-c3ccc4c5c6sc7ccccc7c6ccc5n(-c5cccc(N(c6ccccc6)c6ccccc6)c5)c4c3)c2)cc1 ; CC1(C)c2ccc(N(c3ccccc3)c3ccccc3)cc2-c2cc(-n3c4ccc(N(c5ccccc5)c5ccccc5)cc4c4c5sc6ccccc6c5ccc43)ccc21]

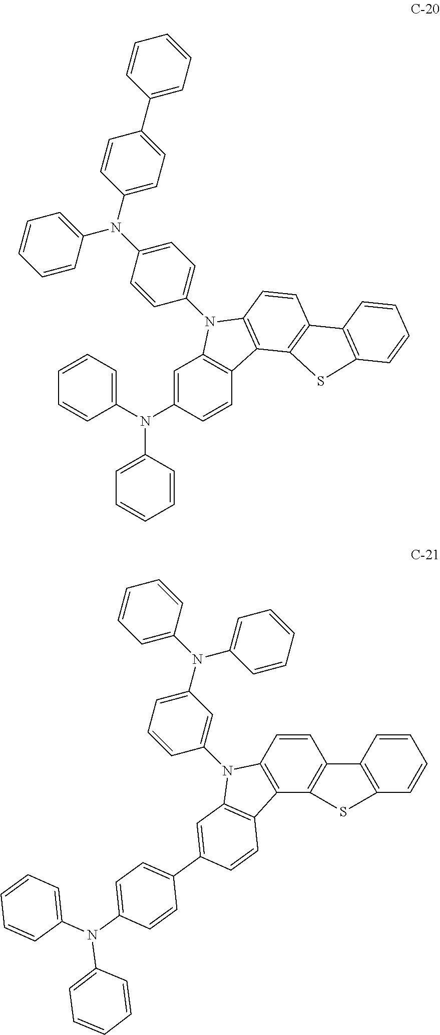 [SMILES: c1ccc(-c2ccc(N(c3ccccc3)c3ccc(-n4c5cc(N(c6ccccc6)c6ccccc6)ccc5c5c6sc7ccccc7c6ccc54)cc3)cc2)cc1 ; c1ccc(N(c2ccccc2)c2ccc(-c3ccc4c5c6sc7ccccc7c6ccc5n(-c5cccc(N(c6ccccc6)c6ccccc6)c5)c4c3)cc2)cc1]

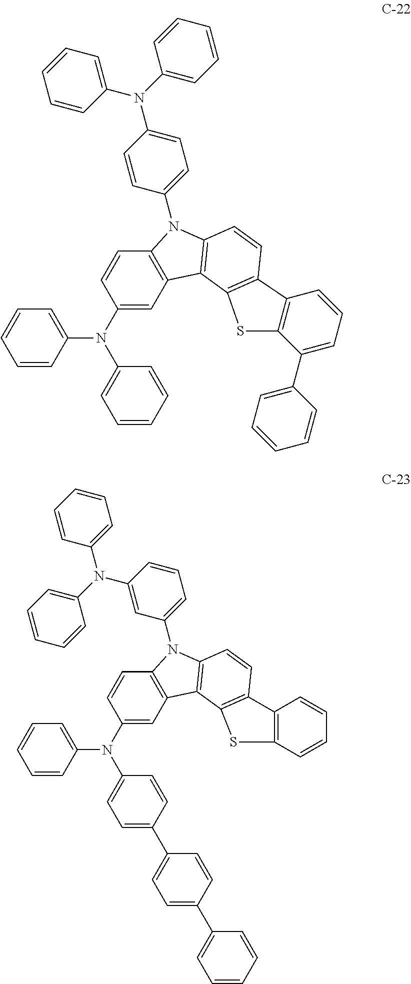 [SMILES: c1ccc(-c2cccc3c2sc2c3ccc3c2c2cc(N(c4ccccc4)c4ccccc4)ccc2n3-c2ccc(N(c3ccccc3)c3ccccc3)cc2)cc1 ; c1ccc(-c2ccc(-c3ccc(N(c4ccccc4)c4ccc5c(c4)c4c6sc7ccccc7c6ccc4n5-c4cccc(N(c5ccccc5)c5ccccc5)c4)cc3)cc2)cc1]

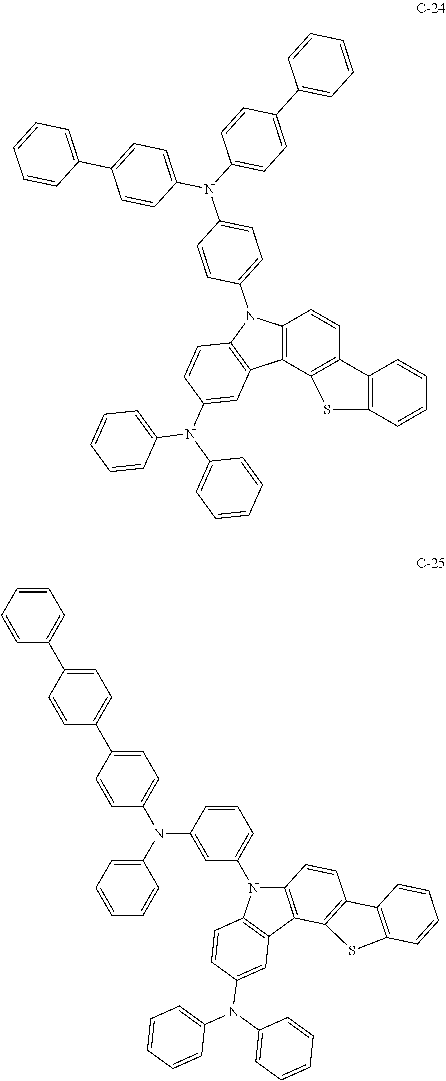 [SMILES: c1ccc(-c2ccc(N(c3ccc(-c4ccccc4)cc3)c3ccc(-n4c5ccc(N(c6ccccc6)c6ccccc6)cc5c5c6sc7ccccc7c6ccc54)cc3)cc2)cc1 ; c1ccc(-c2ccc(-c3ccc(N(c4ccccc4)c4cccc(-n5c6ccc(N(c7ccccc7)c7ccccc7)cc6c6c7sc8ccccc8c7ccc65)c4)cc3)cc2)cc1]

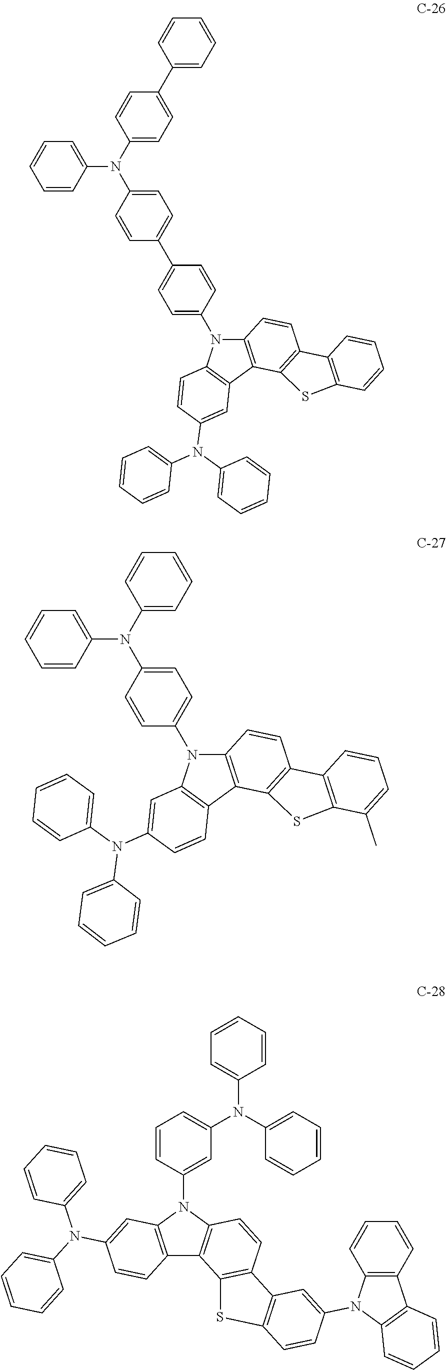 [SMILES: c1ccc(-c2ccc(N(c3ccccc3)c3ccc(-c4ccc(-n5c6ccc(N(c7ccccc7)c7ccccc7)cc6c6c7sc8ccccc8c7ccc65)cc4)cc3)cc2)cc1 ; Cc1cccc2c1sc1c2ccc2c1c1ccc(N(c3ccccc3)c3ccccc3)cc1n2-c1ccc(N(c2ccccc2)c2ccccc2)cc1 ; c1ccc(N(c2ccccc2)c2cccc(-n3c4cc(N(c5ccccc5)c5ccccc5)ccc4c4c5sc6ccc(-n7c8ccccc8c8ccccc87)cc6c5ccc43)c2)cc1]

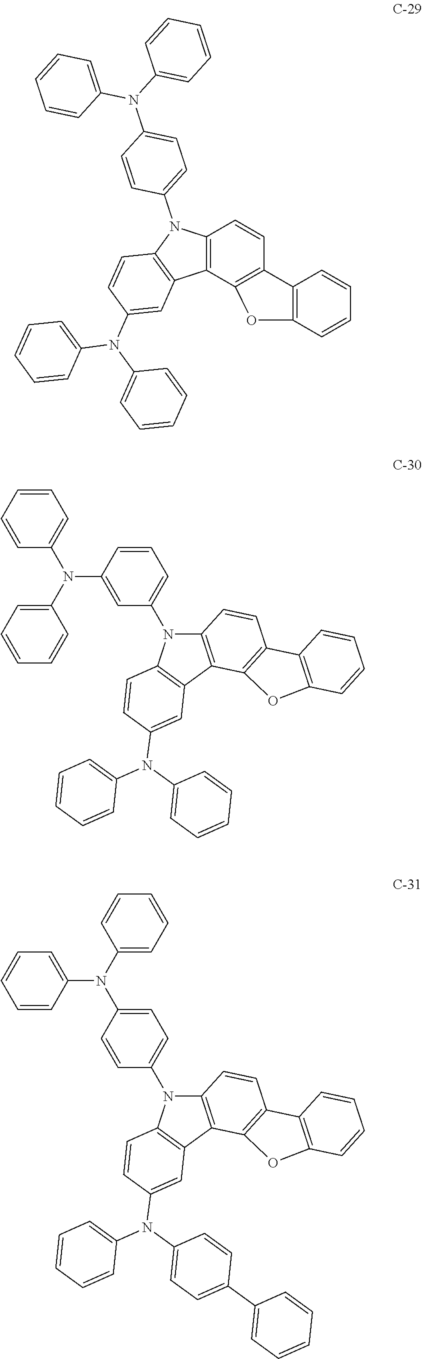 [SMILES: c1ccc(N(c2ccccc2)c2ccc(-n3c4ccc(N(c5ccccc5)c5ccccc5)cc4c4c5oc6ccccc6c5ccc43)cc2)cc1 ; c1ccc(N(c2ccccc2)c2cccc(-n3c4ccc(N(c5ccccc5)c5ccccc5)cc4c4c5oc6ccccc6c5ccc43)c2)cc1 ; c1ccc(-c2ccc(N(c3ccccc3)c3ccc4c(c3)c3c5oc6ccccc6c5ccc3n4-c3ccc(N(c4ccccc4)c4ccccc4)cc3)cc2)cc1]

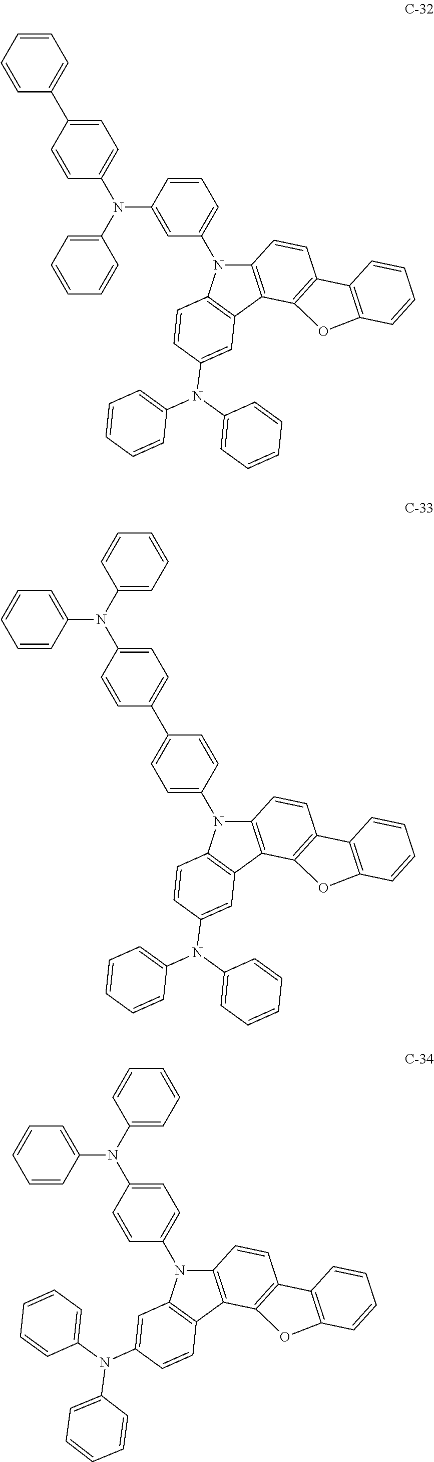 [SMILES: c1ccc(-c2ccc(N(c3ccccc3)c3cccc(-n4c5ccc(N(c6ccccc6)c6ccccc6)cc5c5c6oc7ccccc7c6ccc54)c3)cc2)cc1 ; c1ccc(N(c2ccccc2)c2ccc(-c3ccc(-n4c5ccc(N(c6ccccc6)c6ccccc6)cc5c5c6oc7ccccc7c6ccc54)cc3)cc2)cc1 ; c1ccc(N(c2ccccc2)c2ccc(-n3c4cc(N(c5ccccc5)c5ccccc5)ccc4c4c5oc6ccccc6c5ccc43)cc2)cc1]

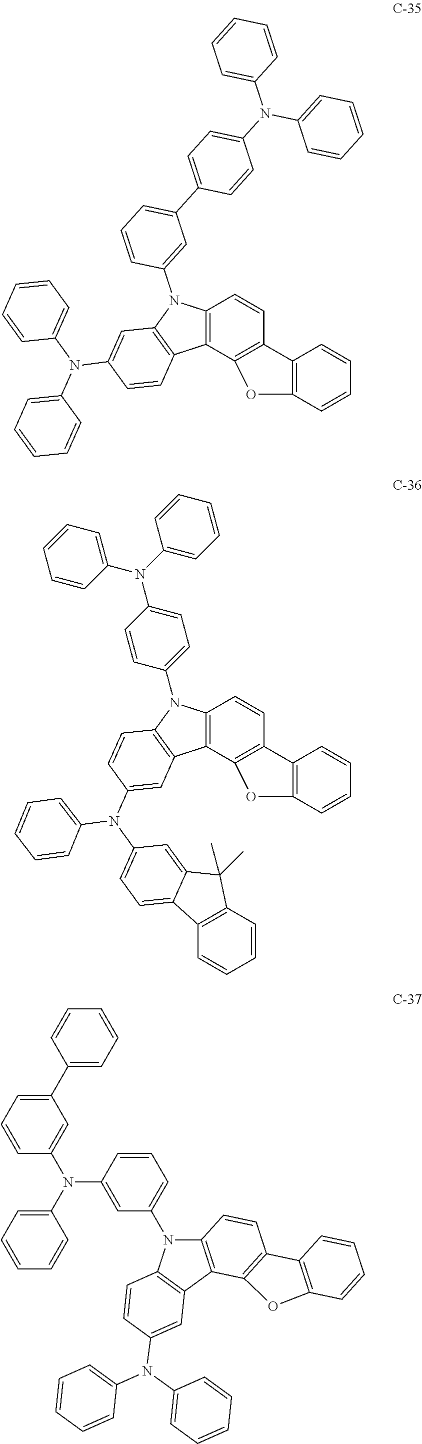 [SMILES: c1ccc(N(c2ccccc2)c2ccc(-c3cccc(-n4c5cc(N(c6ccccc6)c6ccccc6)ccc5c5c6oc7ccccc7c6ccc54)c3)cc2)cc1 ; CC1(C)c2ccccc2-c2ccc(N(c3ccccc3)c3ccc4c(c3)c3c5oc6ccccc6c5ccc3n4-c3ccc(N(c4ccccc4)c4ccccc4)cc3)cc21 ; c1ccc(-c2cccc(N(c3ccccc3)c3cccc(-n4c5ccc(N(c6ccccc6)c6ccccc6)cc5c5c6oc7ccccc7c6ccc54)c3)c2)cc1]

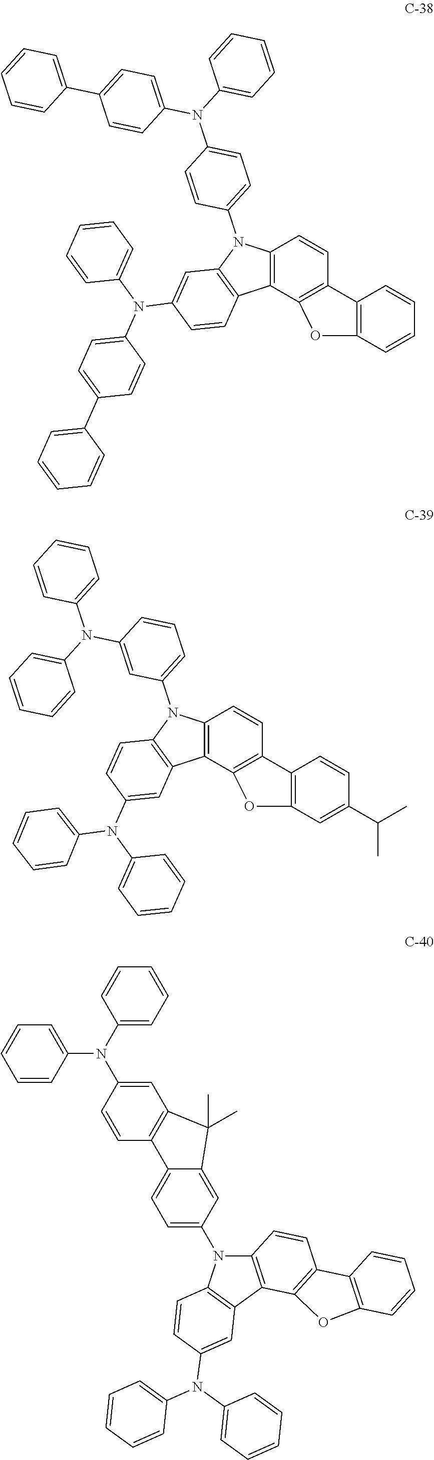 [SMILES: c1ccc(-c2ccc(N(c3ccccc3)c3ccc(-n4c5cc(N(c6ccccc6)c6ccc(-c7ccccc7)cc6)ccc5c5c6oc7ccccc7c6ccc54)cc3)cc2)cc1 ; CC(C)c1ccc2c(c1)oc1c2ccc2c1c1cc(N(c3ccccc3)c3ccccc3)ccc1n2-c1cccc(N(c2ccccc2)c2ccccc2)c1 ; CC1(C)c2cc(N(c3ccccc3)c3ccccc3)ccc2-c2ccc(-n3c4ccc(N(c5ccccc5)c5ccccc5)cc4c4c5oc6ccccc6c5ccc43)cc21]

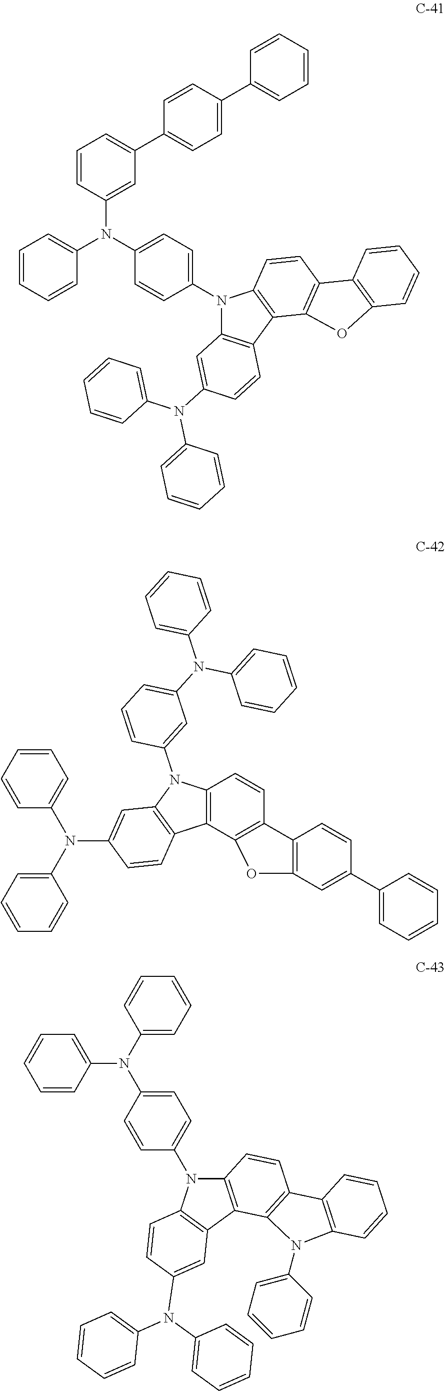 [SMILES: c1ccc(-c2ccc(-c3cccc(N(c4ccccc4)c4ccc(-n5c6cc(N(c7ccccc7)c7ccccc7)ccc6c6c7oc8ccccc8c7ccc65)cc4)c3)cc2)cc1 ; c1ccc(-c2ccc3c(c2)oc2c3ccc3c2c2ccc(N(c4ccccc4)c4ccccc4)cc2n3-c2cccc(N(c3ccccc3)c3ccccc3)c2)cc1 ; c1ccc(N(c2ccccc2)c2ccc(-n3c4ccc(N(c5ccccc5)c5ccccc5)cc4c4c3ccc3c5ccccc5n(-c5ccccc5)c34)cc2)cc1]

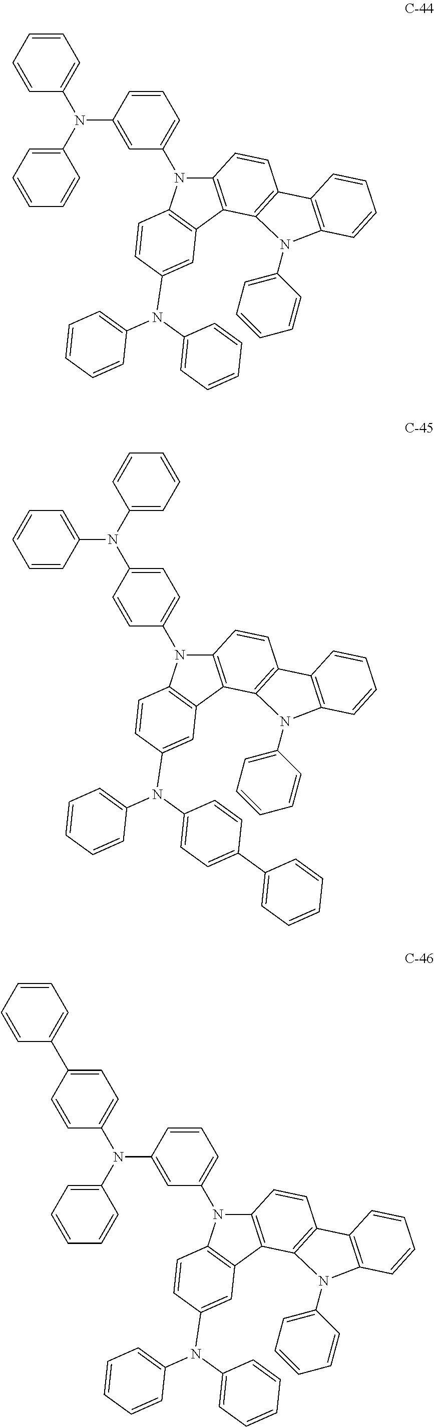 [SMILES: c1ccc(N(c2ccccc2)c2cccc(-n3c4ccc(N(c5ccccc5)c5ccccc5)cc4c4c3ccc3c5ccccc5n(-c5ccccc5)c34)c2)cc1 ; c1ccc(-c2ccc(N(c3ccccc3)c3ccc4c(c3)c3c(ccc5c6ccccc6n(-c6ccccc6)c53)n4-c3ccc(N(c4ccccc4)c4ccccc4)cc3)cc2)cc1 ; c1ccc(-c2ccc(N(c3ccccc3)c3cccc(-n4c5ccc(N(c6ccccc6)c6ccccc6)cc5c5c4ccc4c6ccccc6n(-c6ccccc6)c45)c3)cc2)cc1]

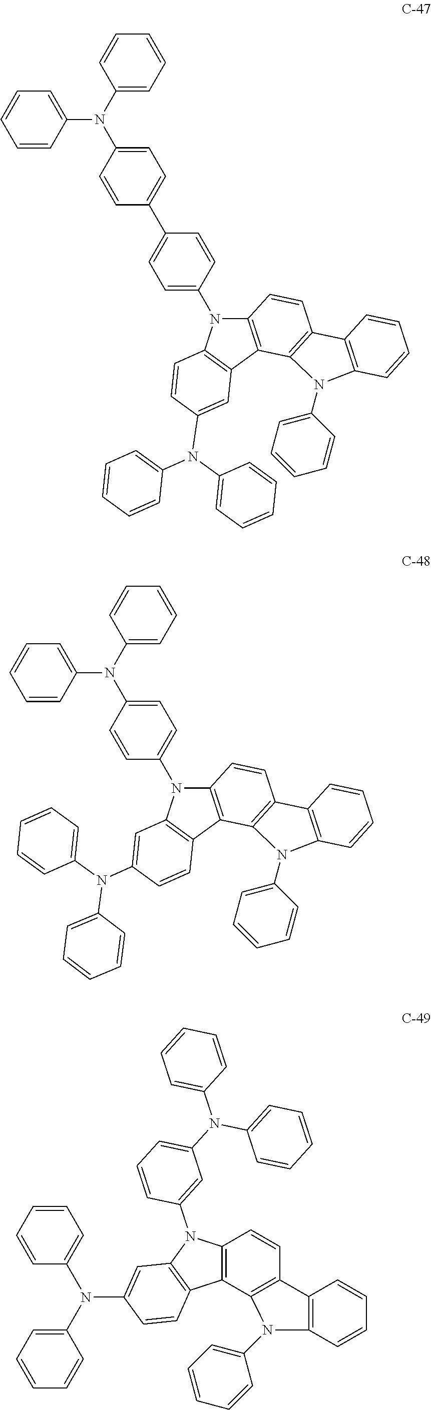 [SMILES: c1ccc(N(c2ccccc2)c2ccc(-c3ccc(-n4c5ccc(N(c6ccccc6)c6ccccc6)cc5c5c4ccc4c6ccccc6n(-c6ccccc6)c45)cc3)cc2)cc1 ; c1ccc(N(c2ccccc2)c2ccc(-n3c4cc(N(c5ccccc5)c5ccccc5)ccc4c4c3ccc3c5ccccc5n(-c5ccccc5)c34)cc2)cc1 ; c1ccc(N(c2ccccc2)c2cccc(-n3c4cc(N(c5ccccc5)c5ccccc5)ccc4c4c3ccc3c5ccccc5n(-c5ccccc5)c34)c2)cc1]

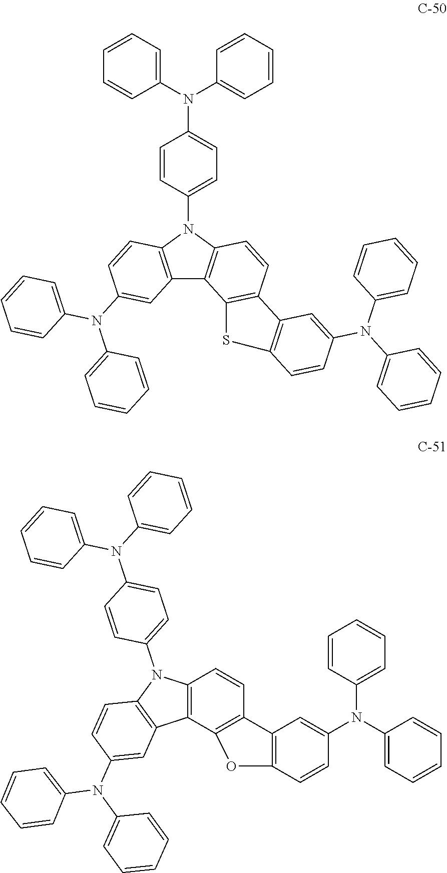 [SMILES: c1ccc(N(c2ccccc2)c2ccc(-n3c4ccc(N(c5ccccc5)c5ccccc5)cc4c4c5sc6ccc(N(c7ccccc7)c7ccccc7)cc6c5ccc43)cc2)cc1 ; c1ccc(N(c2ccccc2)c2ccc(-n3c4ccc(N(c5ccccc5)c5ccccc5)cc4c4c5oc6ccc(N(c7ccccc7)c7ccccc7)cc6c5ccc43)cc2)cc1]

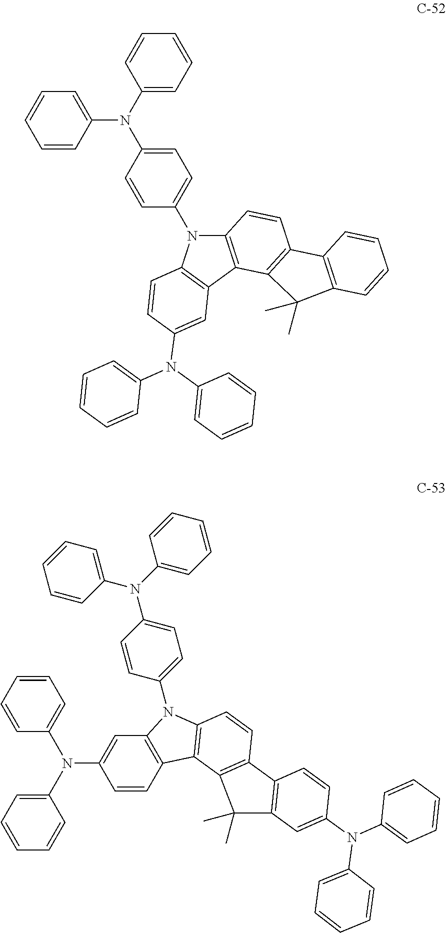 [SMILES: CC1(C)c2ccccc2-c2ccc3c(c21)c1cc(N(c2ccccc2)c2ccccc2)ccc1n3-c1ccc(N(c2ccccc2)c2ccccc2)cc1 ; CC1(C)c2cc(N(c3ccccc3)c3ccccc3)ccc2-c2ccc3c(c21)c1ccc(N(c2ccccc2)c2ccccc2)cc1n3-c1ccc(N(c2ccccc2)c2ccccc2)cc1]

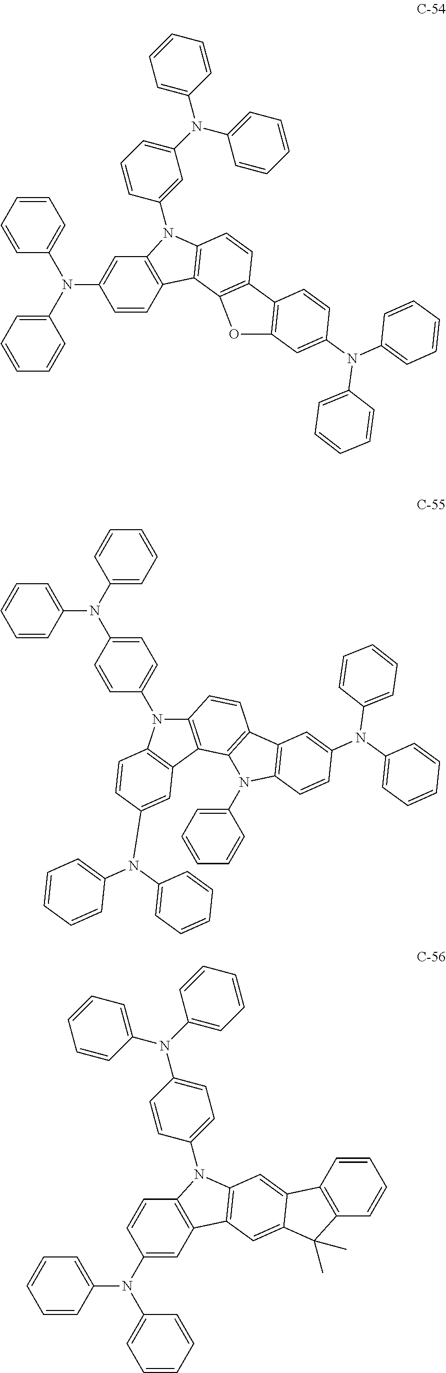

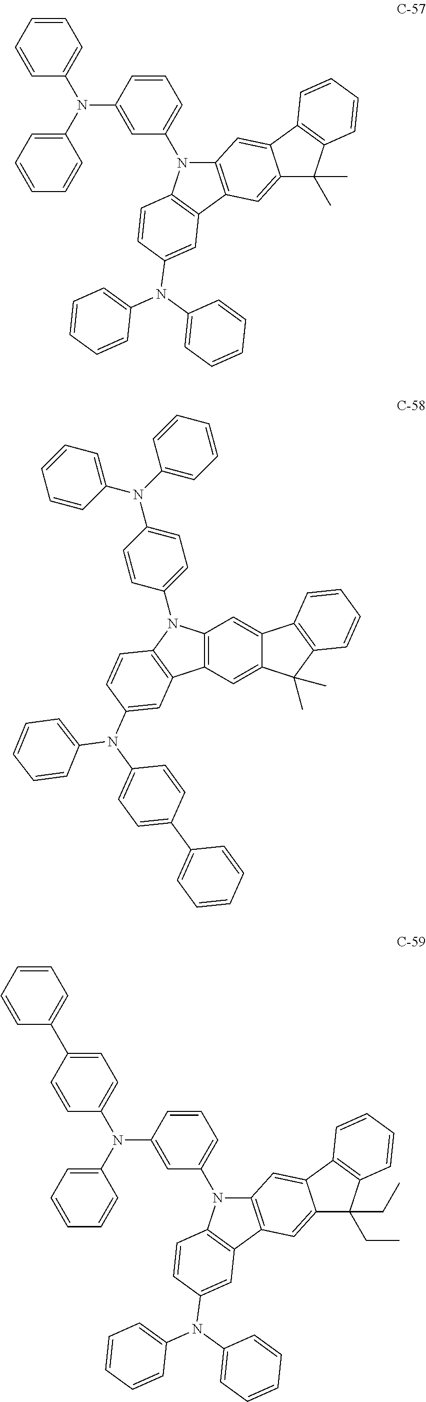 [SMILES: CC1(C)c2ccccc2-c2cc3c(cc21)c1cc(N(c2ccccc2)c2ccccc2)ccc1n3-c1cccc(N(c2ccccc2)c2ccccc2)c1 ; CC1(C)c2ccccc2-c2cc3c(cc21)c1cc(N(c2ccccc2)c2ccc(-c4ccccc4)cc2)ccc1n3-c1ccc(N(c2ccccc2)c2ccccc2)cc1 ; CCC1(CC)c2ccccc2-c2cc3c(cc21)c1cc(N(c2ccccc2)c2ccccc2)ccc1n3-c1cccc(N(c2ccccc2)c2ccc(-c3ccccc3)cc2)c1]

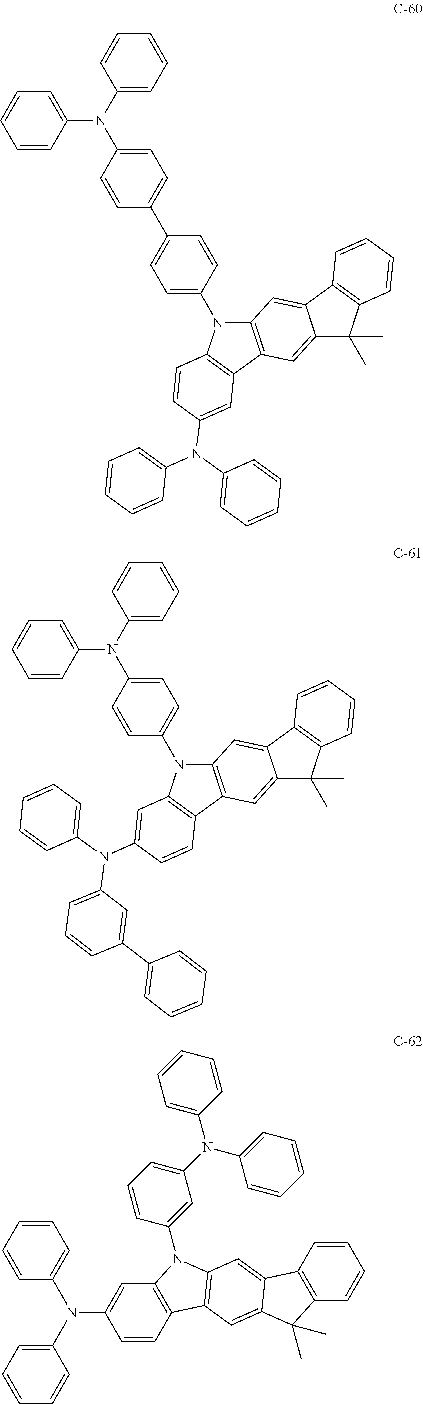

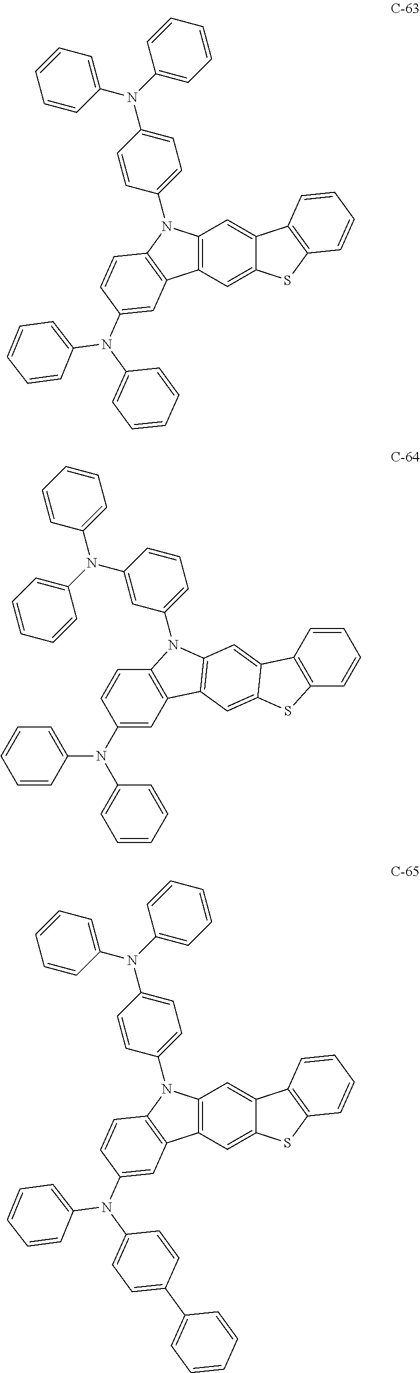

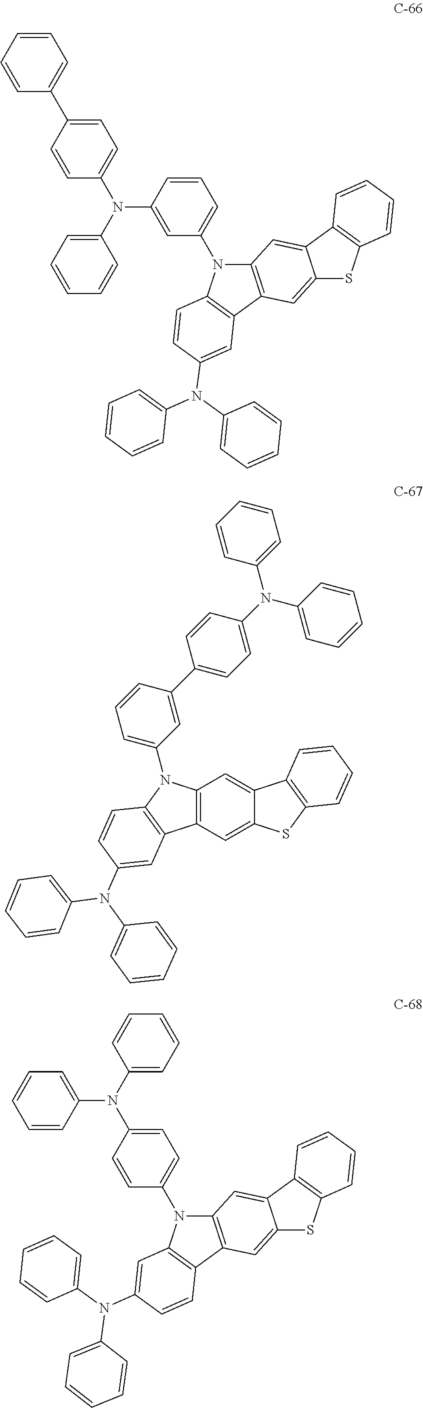

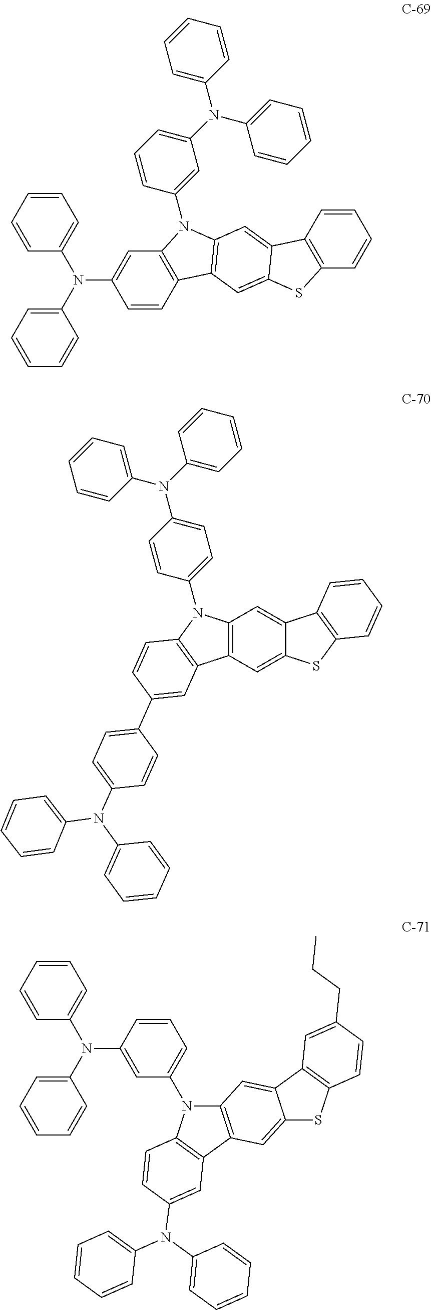 [SMILES: c1ccc(N(c2ccccc2)c2cccc(-n3c4cc(N(c5ccccc5)c5ccccc5)ccc4c4cc5sc6ccccc6c5cc43)c2)cc1 ; c1ccc(N(c2ccccc2)c2ccc(-c3ccc4c(c3)c3cc5sc6ccccc6c5cc3n4-c3ccc(N(c4ccccc4)c4ccccc4)cc3)cc2)cc1 ; CCCc1ccc2sc3cc4c5cc(N(c6ccccc6)c6ccccc6)ccc5n(-c5cccc(N(c6ccccc6)c6ccccc6)c5)c4cc3c2c1]

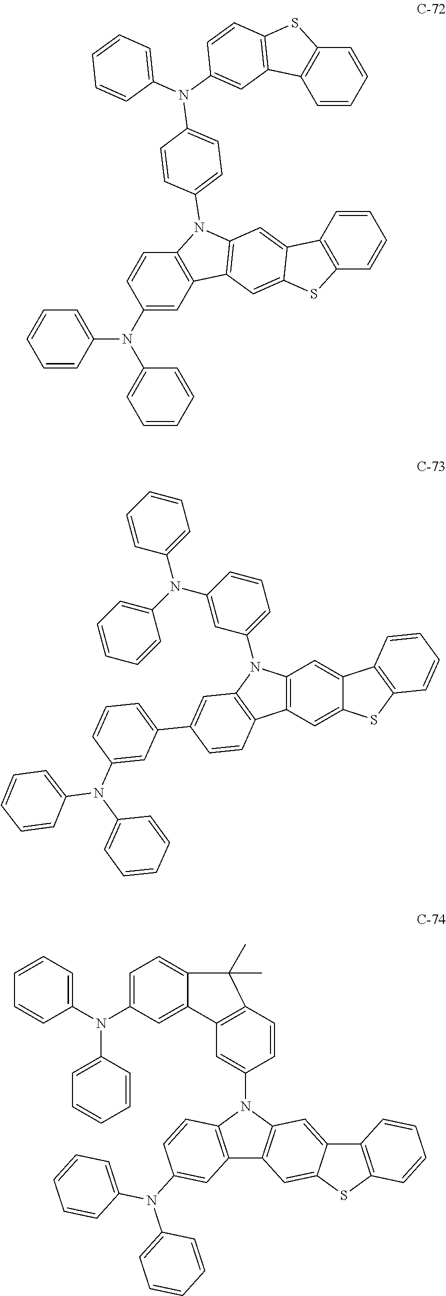 [SMILES: c1ccc(N(c2ccc(-n3c4ccc(N(c5ccccc5)c5ccccc5)cc4c4cc5sc6ccccc6c5cc43)cc2)c2ccc3sc4ccccc4c3c2)cc1 ; c1ccc(N(c2ccccc2)c2cccc(-c3ccc4c5cc6sc7ccccc7c6cc5n(-c5cccc(N(c6ccccc6)c6ccccc6)c5)c4c3)c2)cc1 ; CC1(C)c2ccc(N(c3ccccc3)c3ccccc3)cc2-c2cc(-n3c4ccc(N(c5ccccc5)c5ccccc5)cc4c4cc5sc6ccccc6c5cc43)ccc21]

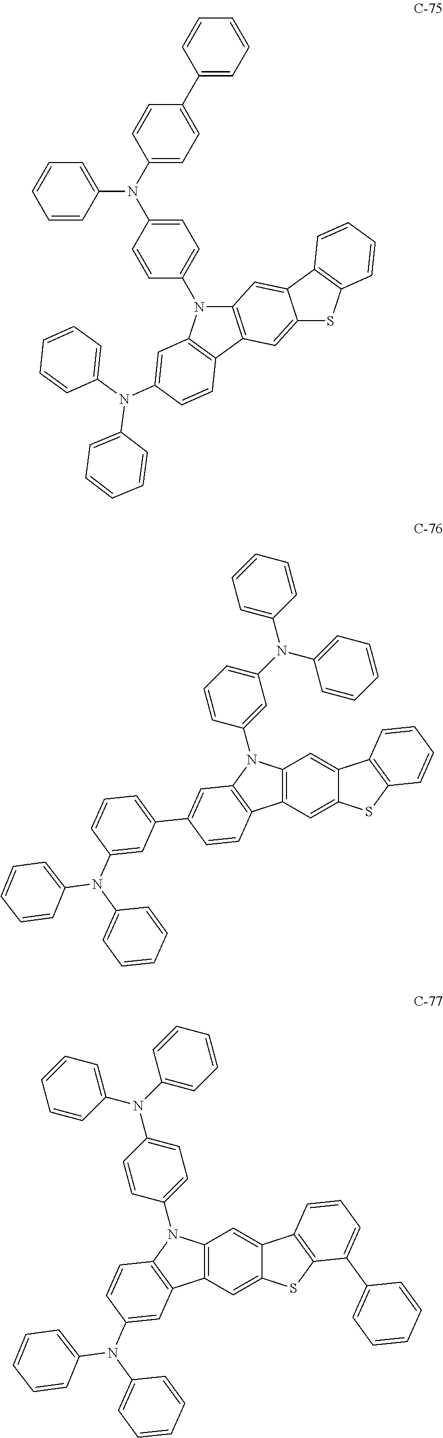 [SMILES: c1ccc(-c2ccc(N(c3ccccc3)c3ccc(-n4c5cc(N(c6ccccc6)c6ccccc6)ccc5c5cc6sc7ccccc7c6cc54)cc3)cc2)cc1 ; c1ccc(N(c2ccccc2)c2cccc(-c3ccc4c5cc6sc7ccccc7c6cc5n(-c5cccc(N(c6ccccc6)c6ccccc6)c5)c4c3)c2)cc1 ; c1ccc(-c2cccc3c2sc2cc4c5cc(N(c6ccccc6)c6ccccc6)ccc5n(-c5ccc(N(c6ccccc6)c6ccccc6)cc5)c4cc23)cc1]

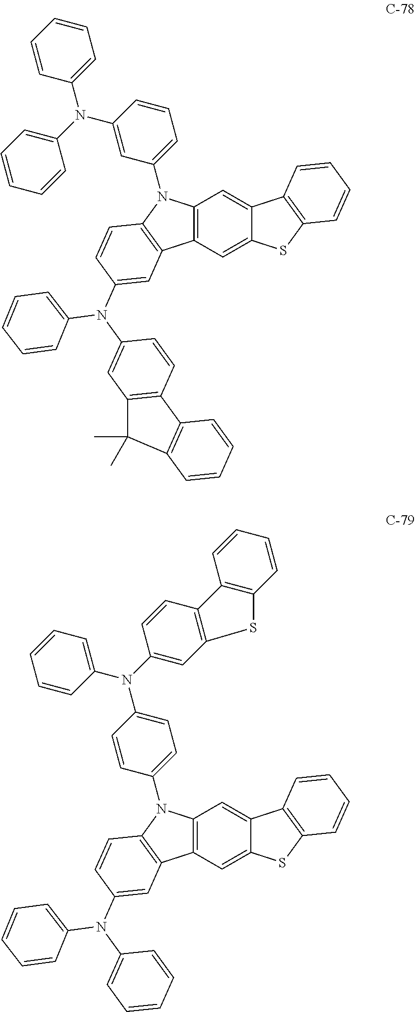 [SMILES: CC1(C)c2ccccc2-c2ccc(N(c3ccccc3)c3ccc4c(c3)c3cc5sc6ccccc6c5cc3n4-c3cccc(N(c4ccccc4)c4ccccc4)c3)cc21 ; c1ccc(N(c2ccc(-n3c4ccc(N(c5ccccc5)c5ccccc5)cc4c4cc5sc6ccccc6c5cc43)cc2)c2ccc3c(c2)sc2ccccc23)cc1]

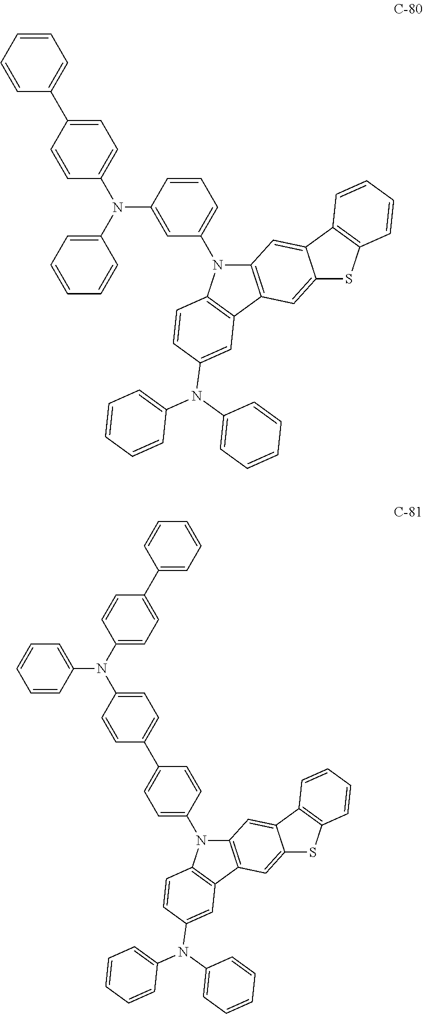 [SMILES: c1ccc(-c2ccc(N(c3ccccc3)c3cccc(-n4c5ccc(N(c6ccccc6)c6ccccc6)cc5c5cc6sc7ccccc7c6cc54)c3)cc2)cc1 ; c1ccc(-c2ccc(N(c3ccccc3)c3ccc(-c4ccc(-n5c6ccc(N(c7ccccc7)c7ccccc7)cc6c6cc7sc8ccccc8c7cc65)cc4)cc3)cc2)cc1]

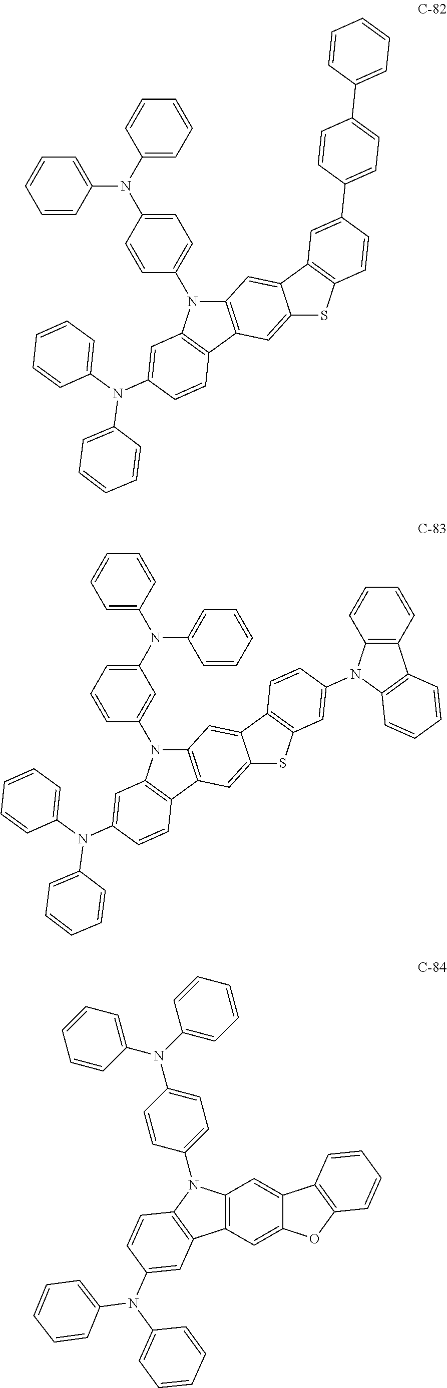 [SMILES: c1ccc(-c2ccc(-c3ccc4sc5cc6c7ccc(N(c8ccccc8)c8ccccc8)cc7n(-c7ccc(N(c8ccccc8)c8ccccc8)cc7)c6cc5c4c3)cc2)cc1 ; c1ccc(N(c2ccccc2)c2cccc(-n3c4cc(N(c5ccccc5)c5ccccc5)ccc4c4cc5sc6cc(-n7c8ccccc8c8ccccc87)ccc6c5cc43)c2)cc1 ; c1ccc(N(c2ccccc2)c2ccc(-n3c4ccc(N(c5ccccc5)c5ccccc5)cc4c4cc5oc6ccccc6c5cc43)cc2)cc1]

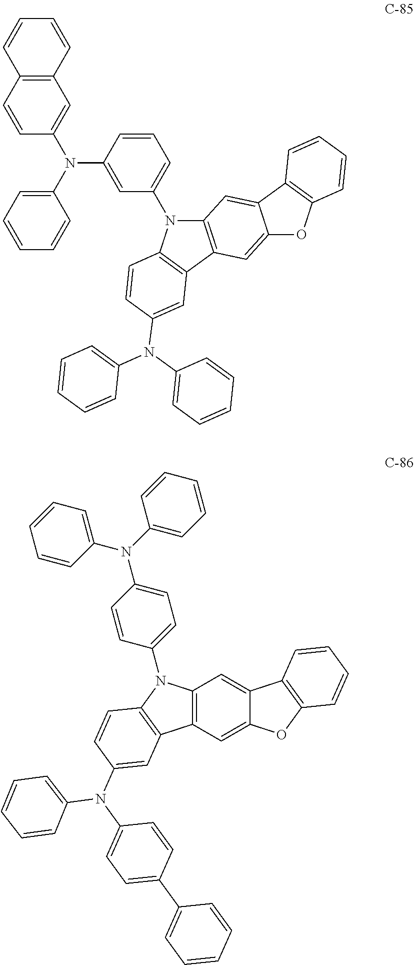 [SMILES: c1ccc(N(c2cccc(-n3c4ccc(N(c5ccccc5)c5ccccc5)cc4c4cc5oc6ccccc6c5cc43)c2)c2ccc3ccccc3c2)cc1 ; c1ccc(-c2ccc(N(c3ccccc3)c3ccc4c(c3)c3cc5oc6ccccc6c5cc3n4-c3ccc(N(c4ccccc4)c4ccccc4)cc3)cc2)cc1]

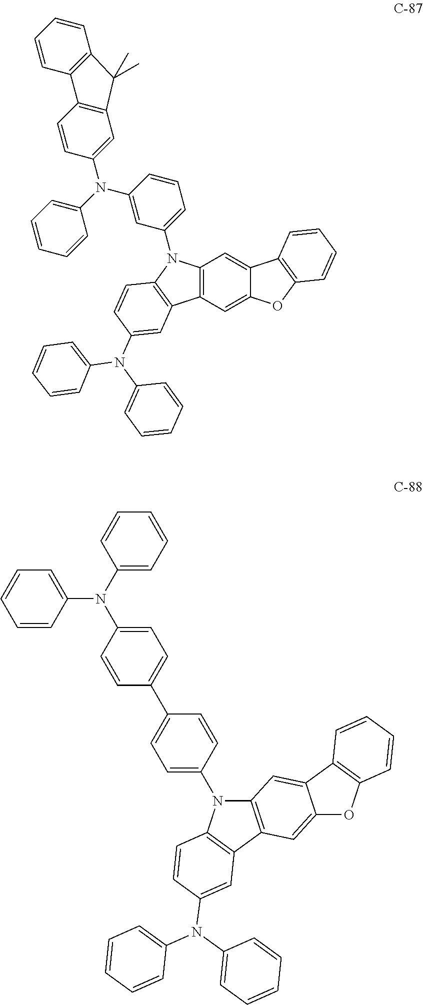 [SMILES: CC1(C)c2ccccc2-c2ccc(N(c3ccccc3)c3cccc(-n4c5ccc(N(c6ccccc6)c6ccccc6)cc5c5cc6oc7ccccc7c6cc54)c3)cc21 ; c1ccc(N(c2ccccc2)c2ccc(-c3ccc(-n4c5ccc(N(c6ccccc6)c6ccccc6)cc5c5cc6oc7ccccc7c6cc54)cc3)cc2)cc1]

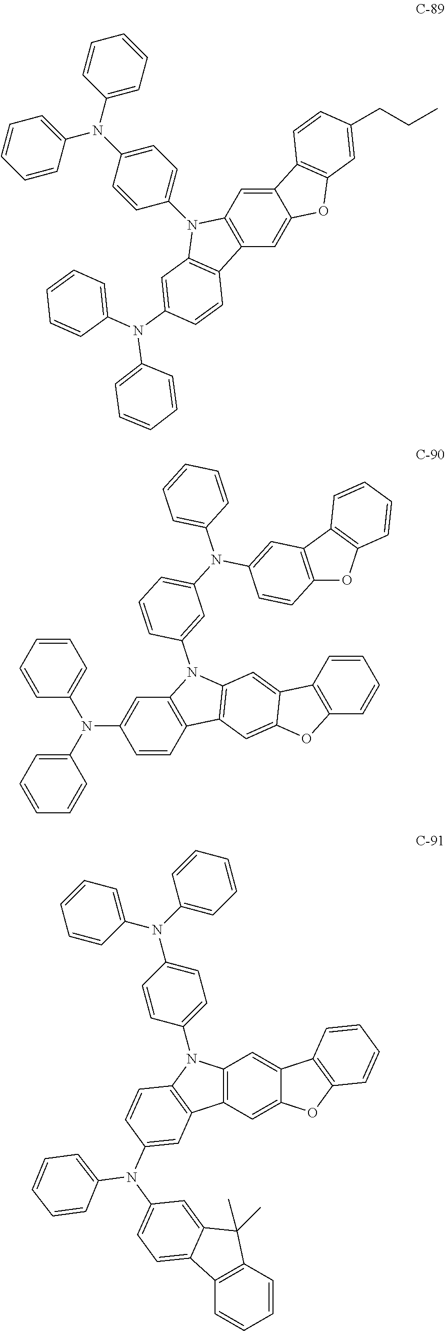 [SMILES: CCCc1ccc2c(c1)oc1cc3c4ccc(N(c5ccccc5)c5ccccc5)cc4n(-c4ccc(N(c5ccccc5)c5ccccc5)cc4)c3cc12 ; c1ccc(N(c2cccc(-n3c4cc(N(c5ccccc5)c5ccccc5)ccc4c4cc5oc6ccccc6c5cc43)c2)c2ccc3oc4ccccc4c3c2)cc1 ; CC1(C)c2ccccc2-c2ccc(N(c3ccccc3)c3ccc4c(c3)c3cc5oc6ccccc6c5cc3n4-c3ccc(N(c4ccccc4)c4ccccc4)cc3)cc21]

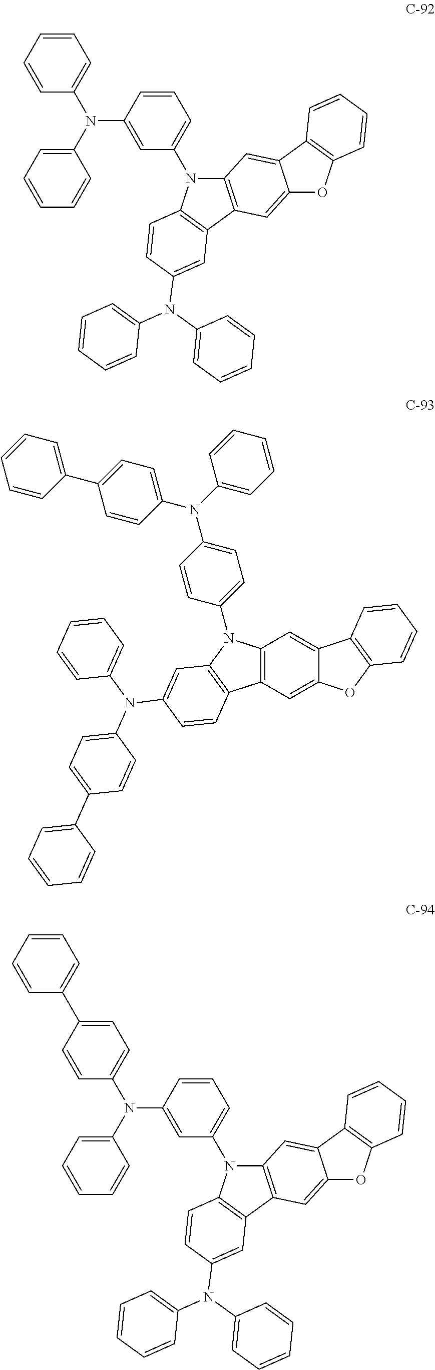 [SMILES: c1ccc(N(c2ccccc2)c2cccc(-n3c4ccc(N(c5ccccc5)c5ccccc5)cc4c4cc5oc6ccccc6c5cc43)c2)cc1 ; c1ccc(-c2ccc(N(c3ccccc3)c3ccc(-n4c5cc(N(c6ccccc6)c6ccc(-c7ccccc7)cc6)ccc5c5cc6oc7ccccc7c6cc54)cc3)cc2)cc1 ; c1ccc(-c2ccc(N(c3ccccc3)c3cccc(-n4c5ccc(N(c6ccccc6)c6ccccc6)cc5c5cc6oc7ccccc7c6cc54)c3)cc2)cc1]

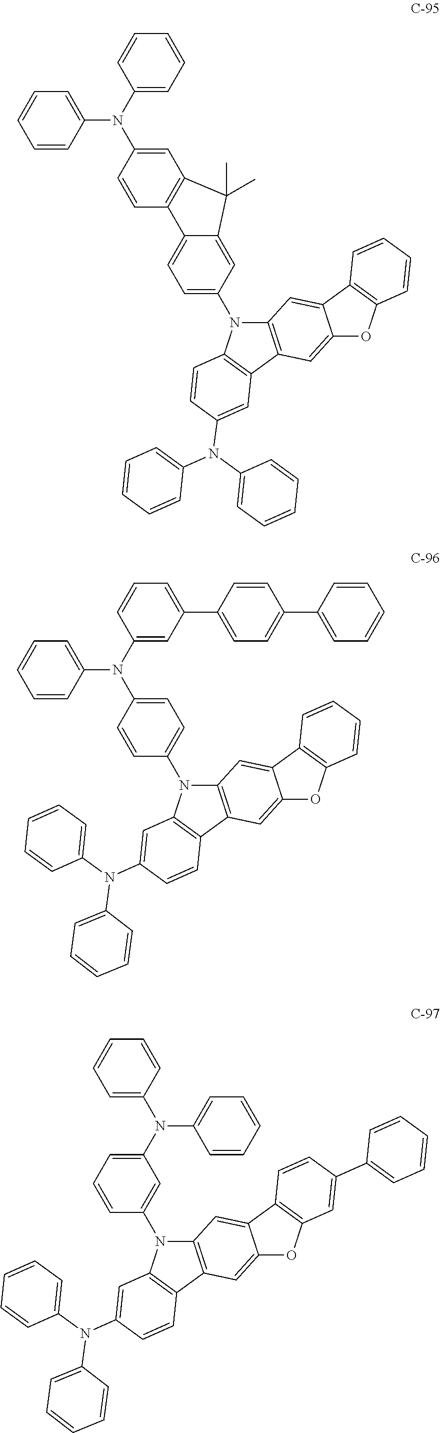 [SMILES: CC1(C)c2cc(N(c3ccccc3)c3ccccc3)ccc2-c2ccc(-n3c4ccc(N(c5ccccc5)c5ccccc5)cc4c4cc5oc6ccccc6c5cc43)cc21 ; c1ccc(-c2ccc(-c3cccc(N(c4ccccc4)c4ccc(-n5c6cc(N(c7ccccc7)c7ccccc7)ccc6c6cc7oc8ccccc8c7cc65)cc4)c3)cc2)cc1 ; c1ccc(-c2ccc3c(c2)oc2cc4c5ccc(N(c6ccccc6)c6ccccc6)cc5n(-c5cccc(N(c6ccccc6)c6ccccc6)c5)c4cc23)cc1]

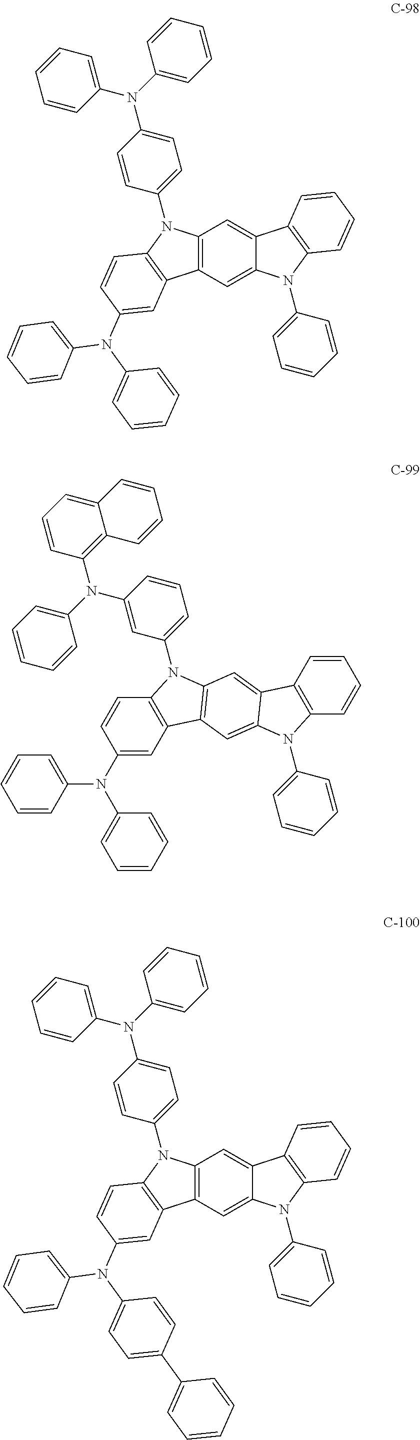 [SMILES: c1ccc(N(c2ccccc2)c2ccc(-n3c4ccc(N(c5ccccc5)c5ccccc5)cc4c4cc5c(cc43)c3ccccc3n5-c3ccccc3)cc2)cc1 ; c1ccc(N(c2ccccc2)c2ccc3c(c2)c2cc4c(cc2n3-c2cccc(N(c3ccccc3)c3cccc5ccccc35)c2)c2ccccc2n4-c2ccccc2)cc1 ; c1ccc(-c2ccc(N(c3ccccc3)c3ccc4c(c3)c3cc5c(cc3n4-c3ccc(N(c4ccccc4)c4ccccc4)cc3)c3ccccc3n5-c3ccccc3)cc2)cc1]

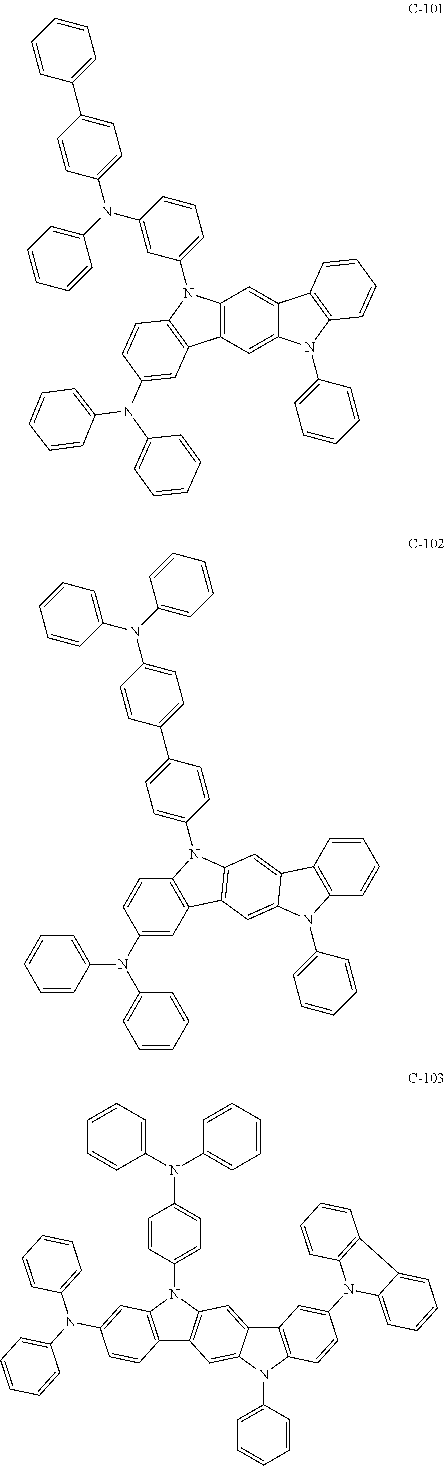 [SMILES: c1ccc(-c2ccc(N(c3ccccc3)c3cccc(-n4c5ccc(N(c6ccccc6)c6ccccc6)cc5c5cc6c(cc54)c4ccccc4n6-c4ccccc4)c3)cc2)cc1 ; c1ccc(N(c2ccccc2)c2ccc(-c3ccc(-n4c5ccc(N(c6ccccc6)c6ccccc6)cc5c5cc6c(cc54)c4ccccc4n6-c4ccccc4)cc3)cc2)cc1 ; c1ccc(N(c2ccccc2)c2ccc(-n3c4cc(N(c5ccccc5)c5ccccc5)ccc4c4cc5c(cc43)c3cc(-n4c6ccccc6c6ccccc64)ccc3n5-c3ccccc3)cc2)cc1]

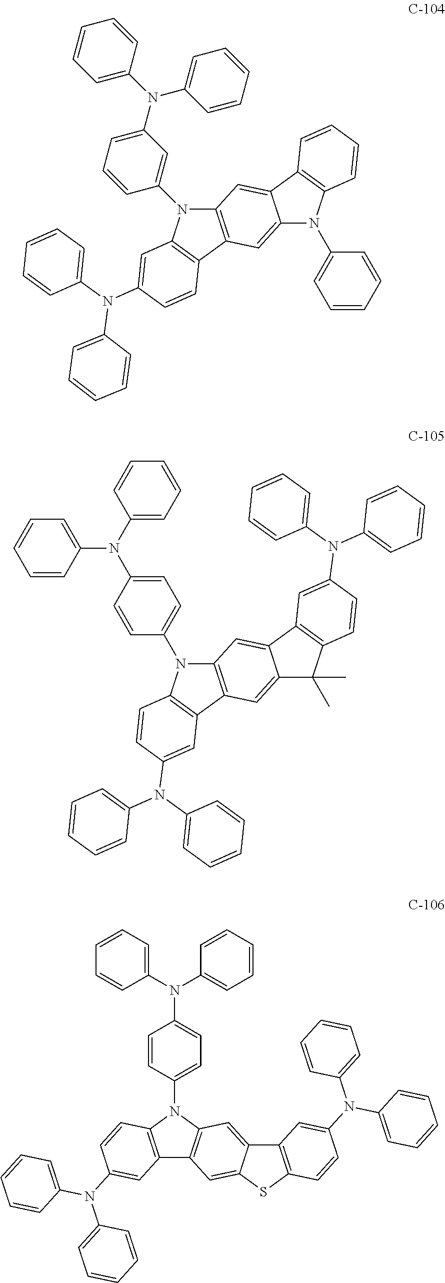 [SMILES: c1ccc(N(c2ccccc2)c2cccc(-n3c4cc(N(c5ccccc5)c5ccccc5)ccc4c4cc5c(cc43)c3ccccc3n5-c3ccccc3)c2)cc1 ; CC1(C)c2ccc(N(c3ccccc3)c3ccccc3)cc2-c2cc3c(cc21)c1cc(N(c2ccccc2)c2ccccc2)ccc1n3-c1ccc(N(c2ccccc2)c2ccccc2)cc1 ; c1ccc(N(c2ccccc2)c2ccc(-n3c4ccc(N(c5ccccc5)c5ccccc5)cc4c4cc5sc6ccc(N(c7ccccc7)c7ccccc7)cc6c5cc43)cc2)cc1]

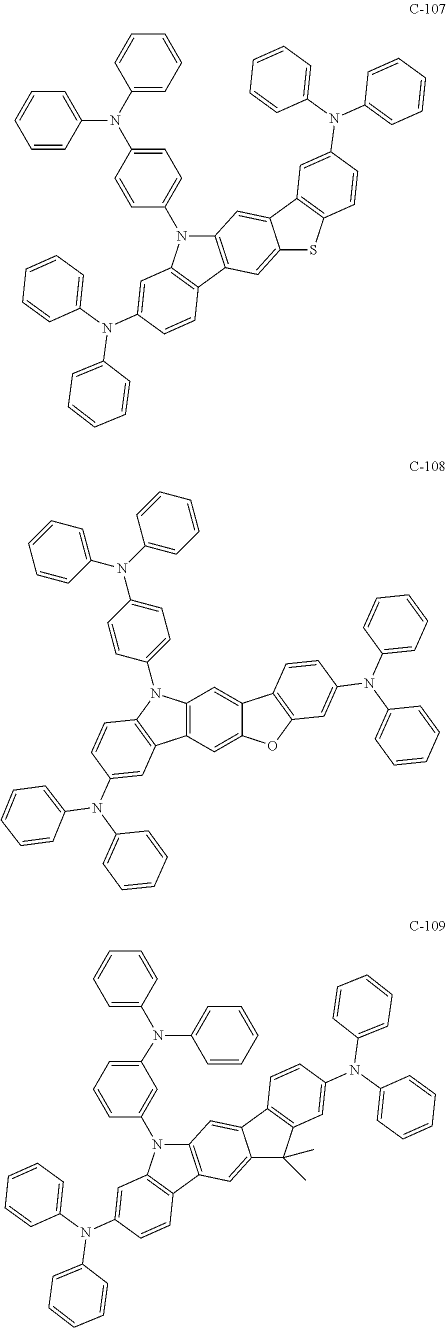 [SMILES: c1ccc(N(c2ccccc2)c2ccc(-n3c4cc(N(c5ccccc5)c5ccccc5)ccc4c4cc5sc6ccc(N(c7ccccc7)c7ccccc7)cc6c5cc43)cc2)cc1 ; c1ccc(N(c2ccccc2)c2ccc(-n3c4ccc(N(c5ccccc5)c5ccccc5)cc4c4cc5oc6cc(N(c7ccccc7)c7ccccc7)ccc6c5cc43)cc2)cc1 ; CC1(C)c2cc(N(c3ccccc3)c3ccccc3)ccc2-c2cc3c(cc21)c1ccc(N(c2ccccc2)c2ccccc2)cc1n3-c1cccc(N(c2ccccc2)c2ccccc2)c1]

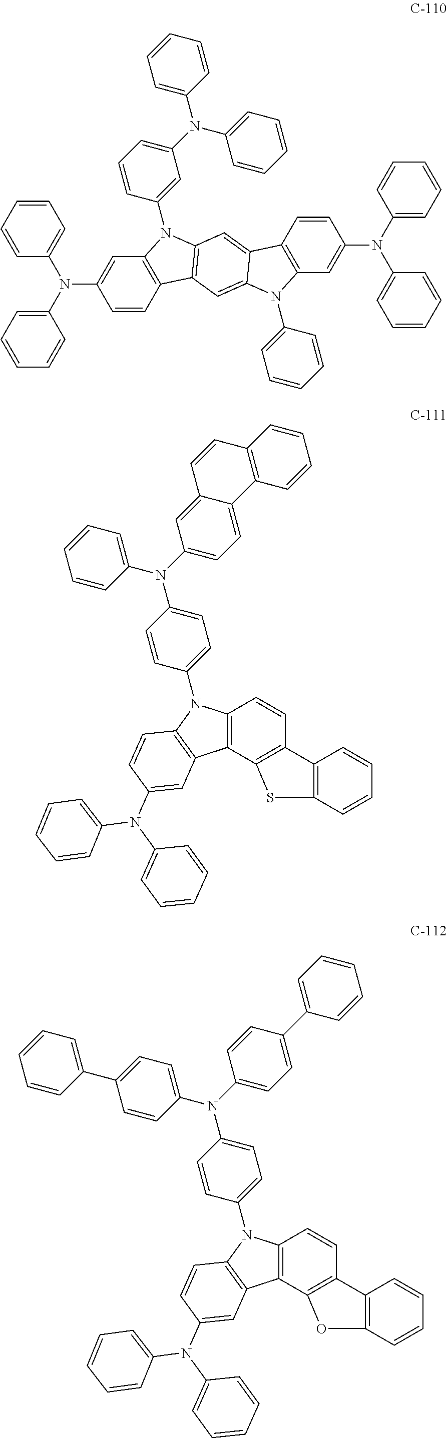

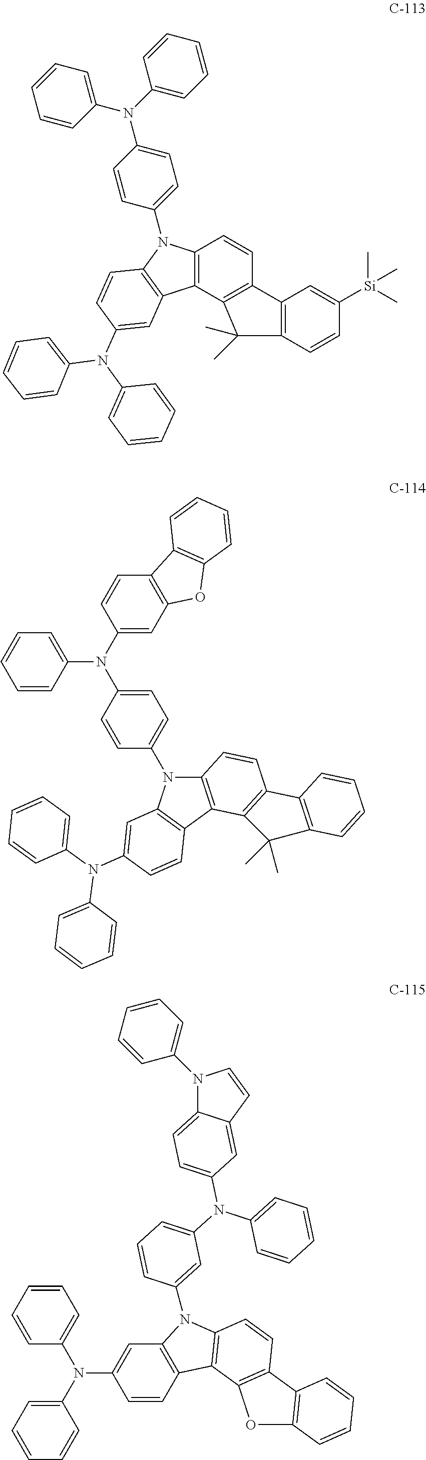 [SMILES: CC1(C)c2ccc([Si](C)(C)C)cc2-c2ccc3c(c21)c1cc(N(c2ccccc2)c2ccccc2)ccc1n3-c1ccc(N(c2ccccc2)c2ccccc2)cc1 ; CC1(C)c2ccccc2-c2ccc3c(c21)c1ccc(N(c2ccccc2)c2ccccc2)cc1n3-c1ccc(N(c2ccccc2)c2ccc3c(c2)oc2ccccc23)cc1 ; c1ccc(N(c2cccc(-n3c4cc(N(c5ccccc5)c5ccccc5)ccc4c4c5oc6ccccc6c5ccc43)c2)c2ccc3c(ccn3-c3ccccc3)c2)cc1]

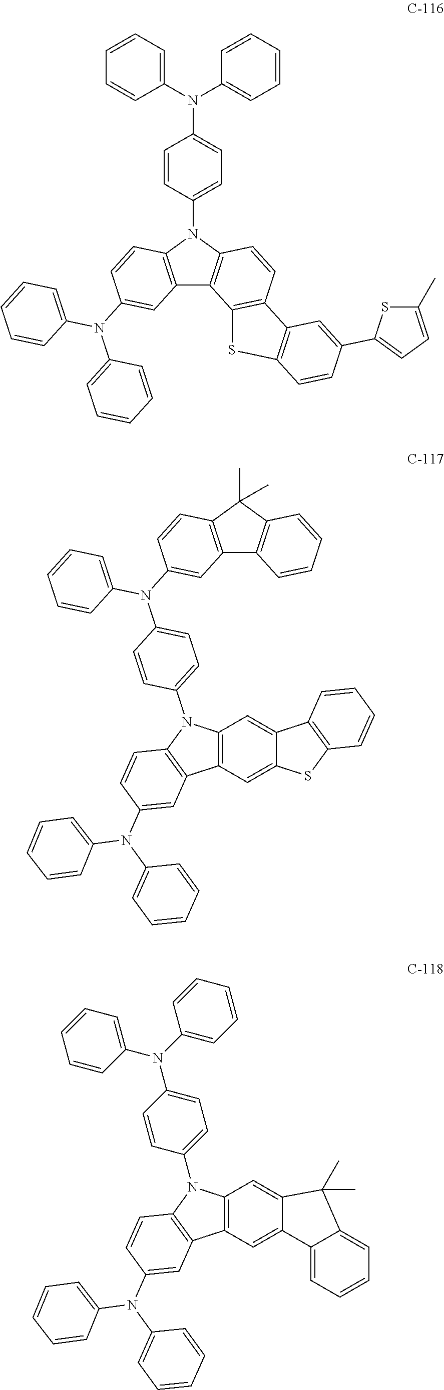 [SMILES: Cc1ccc(-c2ccc3sc4c(ccc5c4c4cc(N(c6ccccc6)c6ccccc6)ccc4n5-c4ccc(N(c5ccccc5)c5ccccc5)cc4)c3c2)s1 ; CC1(C)c2ccccc2-c2cc(N(c3ccccc3)c3ccc(-n4c5ccc(N(c6ccccc6)c6ccccc6)cc5c5cc6sc7ccccc7c6cc54)cc3)ccc21 ; CC1(C)c2ccccc2-c2cc3c4cc(N(c5ccccc5)c5ccccc5)ccc4n(-c4ccc(N(c5ccccc5)c5ccccc5)cc4)c3cc21]

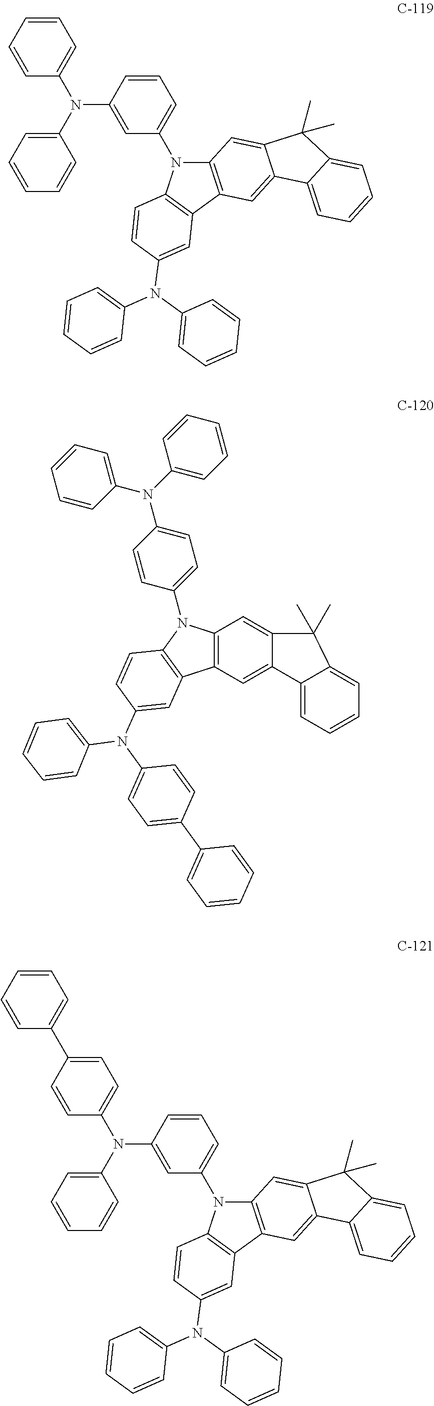 [SMILES: CC1(C)c2ccccc2-c2cc3c4cc(N(c5ccccc5)c5ccccc5)ccc4n(-c4cccc(N(c5ccccc5)c5ccccc5)c4)c3cc21 ; CC1(C)c2ccccc2-c2cc3c4cc(N(c5ccccc5)c5ccc(-c6ccccc6)cc5)ccc4n(-c4ccc(N(c5ccccc5)c5ccccc5)cc4)c3cc21 ; CC1(C)c2ccccc2-c2cc3c4cc(N(c5ccccc5)c5ccccc5)ccc4n(-c4cccc(N(c5ccccc5)c5ccc(-c6ccccc6)cc5)c4)c3cc21]

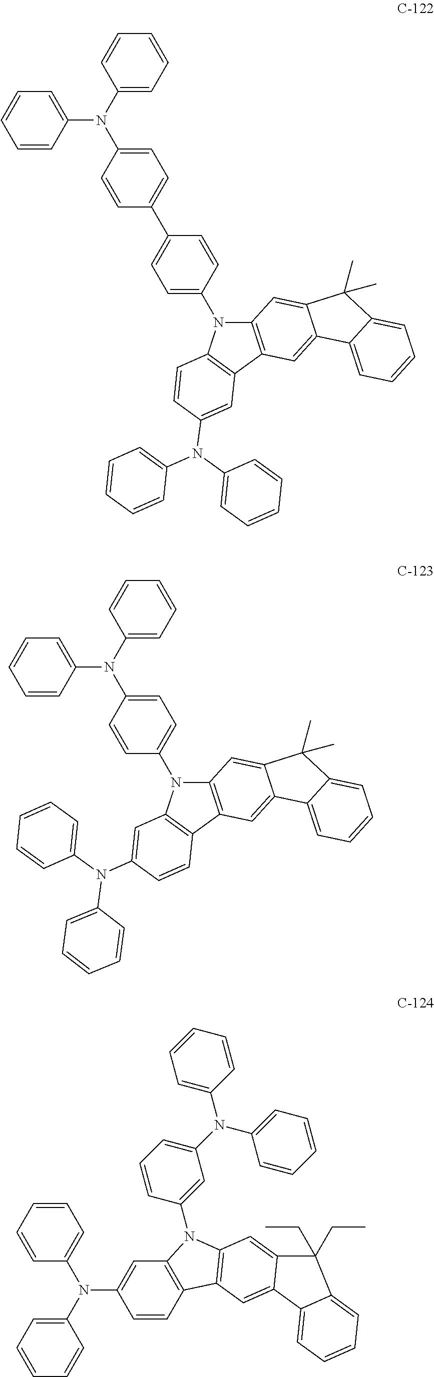 [SMILES: CC1(C)c2ccccc2-c2cc3c4cc(N(c5ccccc5)c5ccccc5)ccc4n(-c4ccc(-c5ccc(N(c6ccccc6)c6ccccc6)cc5)cc4)c3cc21 ; CC1(C)c2ccccc2-c2cc3c4ccc(N(c5ccccc5)c5ccccc5)cc4n(-c4ccc(N(c5ccccc5)c5ccccc5)cc4)c3cc21 ; CCC1(CC)c2ccccc2-c2cc3c4ccc(N(c5ccccc5)c5ccccc5)cc4n(-c4cccc(N(c5ccccc5)c5ccccc5)c4)c3cc21]

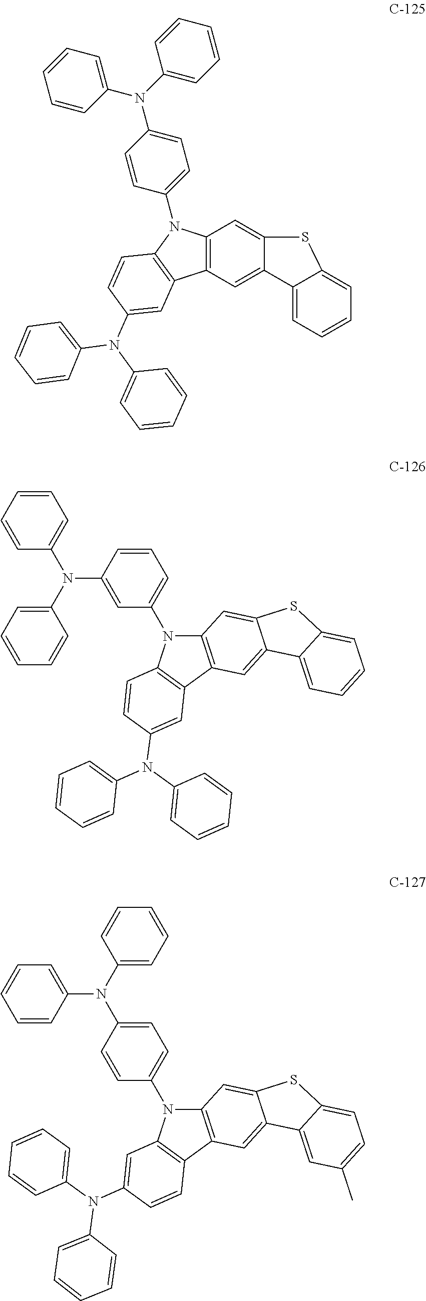 [SMILES: c1ccc(N(c2ccccc2)c2ccc(-n3c4ccc(N(c5ccccc5)c5ccccc5)cc4c4cc5c(cc43)sc3ccccc35)cc2)cc1 ; c1ccc(N(c2ccccc2)c2cccc(-n3c4ccc(N(c5ccccc5)c5ccccc5)cc4c4cc5c(cc43)sc3ccccc35)c2)cc1 ; Cc1ccc2sc3cc4c(cc3c2c1)c1ccc(N(c2ccccc2)c2ccccc2)cc1n4-c1ccc(N(c2ccccc2)c2ccccc2)cc1]

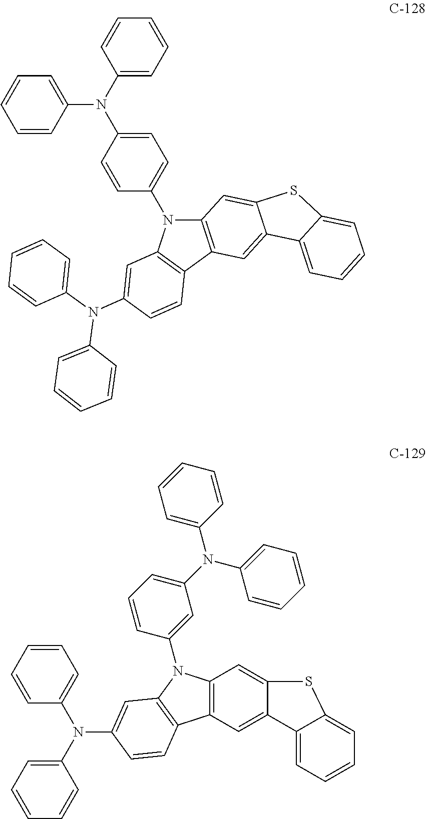 [SMILES: c1ccc(N(c2ccccc2)c2ccc(-n3c4cc(N(c5ccccc5)c5ccccc5)ccc4c4cc5c(cc43)sc3ccccc35)cc2)cc1 ; c1ccc(N(c2ccccc2)c2cccc(-n3c4cc(N(c5ccccc5)c5ccccc5)ccc4c4cc5c(cc43)sc3ccccc35)c2)cc1]

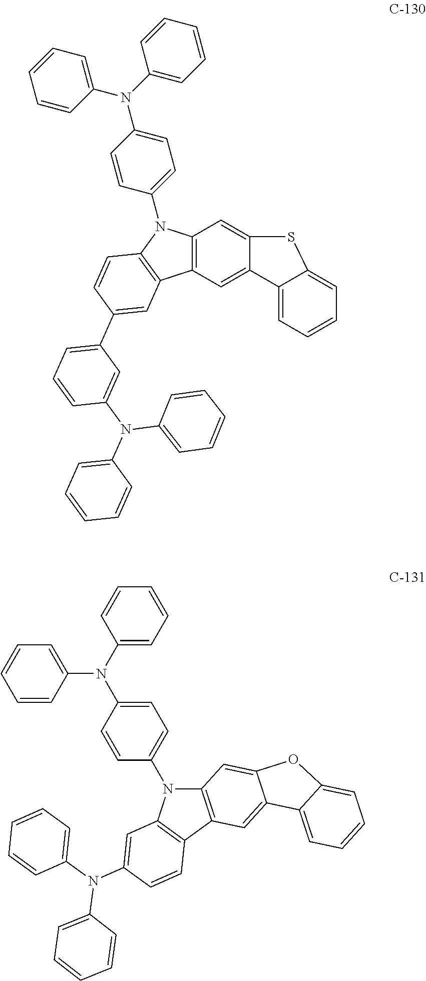 [SMILES: c1ccc(N(c2ccccc2)c2ccc(-n3c4ccc(-c5cccc(N(c6ccccc6)c6ccccc6)c5)cc4c4cc5c(cc43)sc3ccccc35)cc2)cc1 ; c1ccc(N(c2ccccc2)c2ccc(-n3c4cc(N(c5ccccc5)c5ccccc5)ccc4c4cc5c(cc43)oc3ccccc35)cc2)cc1]

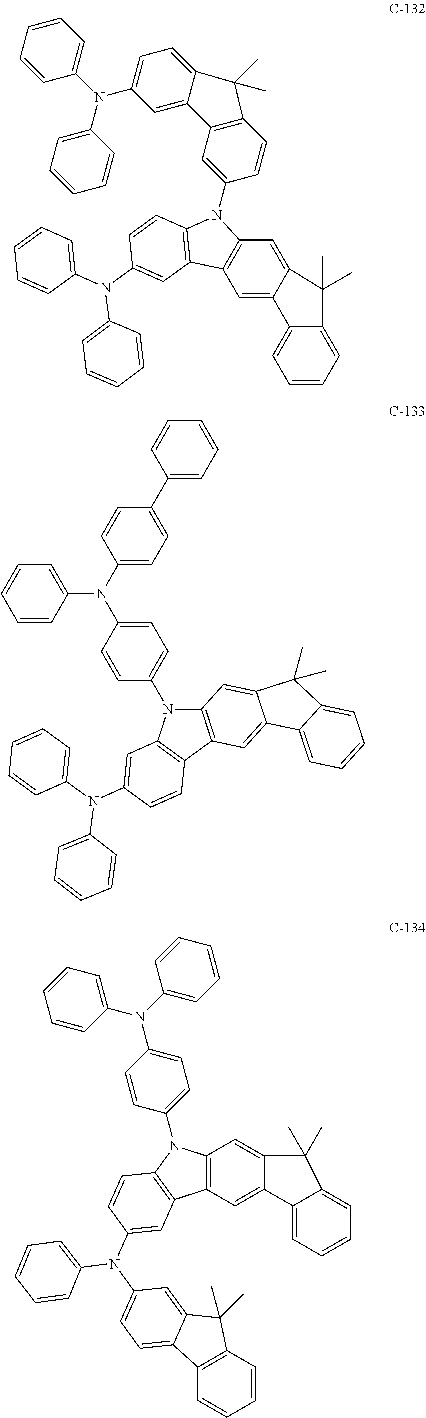 [SMILES: CC1(C)c2ccc(N(c3ccccc3)c3ccccc3)cc2-c2cc(-n3c4ccc(N(c5ccccc5)c5ccccc5)cc4c4cc5c(cc43)C(C)(C)c3ccccc3-5)ccc21 ; CC1(C)c2ccccc2-c2cc3c4ccc(N(c5ccccc5)c5ccccc5)cc4n(-c4ccc(N(c5ccccc5)c5ccc(-c6ccccc6)cc5)cc4)c3cc21 ; CC1(C)c2ccccc2-c2ccc(N(c3ccccc3)c3ccc4c(c3)c3cc5c(cc3n4-c3ccc(N(c4ccccc4)c4ccccc4)cc3)C(C)(C)c3ccccc3-5)cc21]

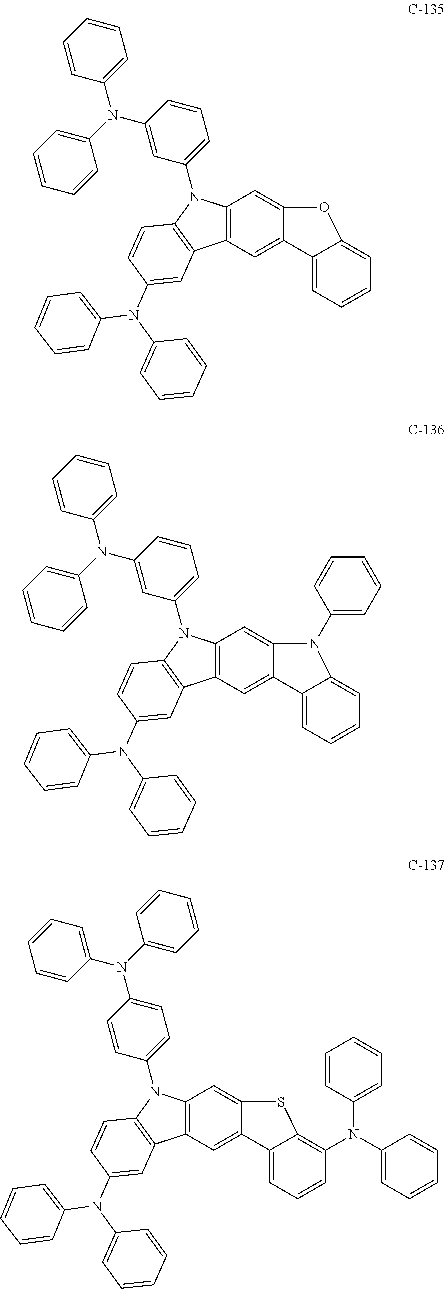 [SMILES: c1ccc(N(c2ccccc2)c2cccc(-n3c4ccc(N(c5ccccc5)c5ccccc5)cc4c4cc5c(cc43)oc3ccccc35)c2)cc1 ; c1ccc(N(c2ccccc2)c2cccc(-n3c4ccc(N(c5ccccc5)c5ccccc5)cc4c4cc5c6ccccc6n(-c6ccccc6)c5cc43)c2)cc1 ; c1ccc(N(c2ccccc2)c2ccc(-n3c4ccc(N(c5ccccc5)c5ccccc5)cc4c4cc5c(cc43)sc3c(N(c4ccccc4)c4ccccc4)cccc35)cc2)cc1]

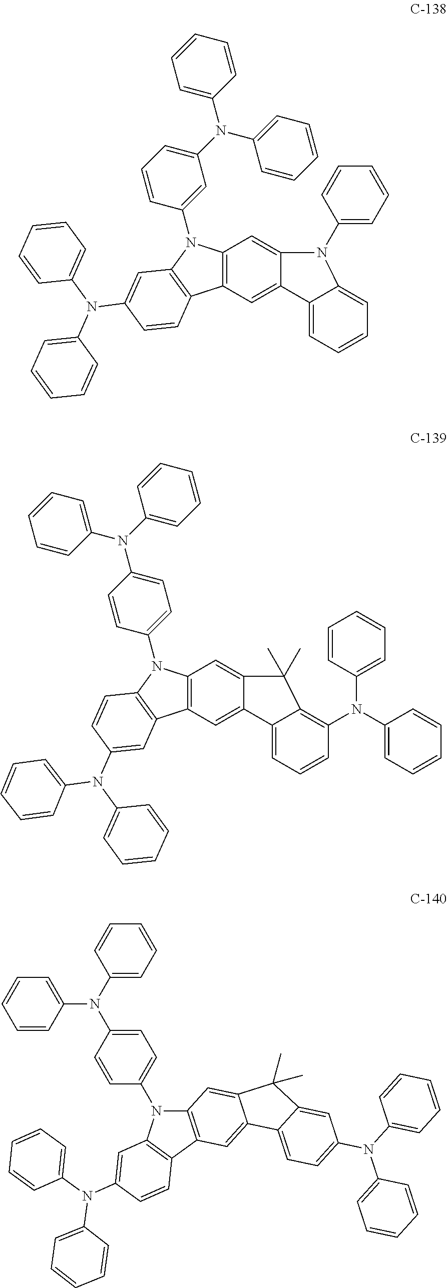 [SMILES: c1ccc(N(c2ccccc2)c2cccc(-n3c4cc(N(c5ccccc5)c5ccccc5)ccc4c4cc5c6ccccc6n(-c6ccccc6)c5cc43)c2)cc1 ; CC1(C)c2cc3c(cc2-c2cccc(N(c4ccccc4)c4ccccc4)c21)c1cc(N(c2ccccc2)c2ccccc2)ccc1n3-c1ccc(N(c2ccccc2)c2ccccc2)cc1 ; CC1(C)c2cc(N(c3ccccc3)c3ccccc3)ccc2-c2cc3c4ccc(N(c5ccccc5)c5ccccc5)cc4n(-c4ccc(N(c5ccccc5)c5ccccc5)cc4)c3cc21]

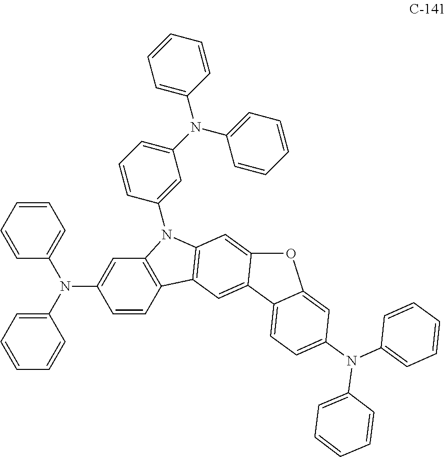 [SMILES: c1ccc(N(c2ccccc2)c2cccc(-n3c4cc(N(c5ccccc5)c5ccccc5)ccc4c4cc5c(cc43)oc3cc(N(c4ccccc4)c4ccccc4)ccc35)c2)cc1]

The compounds of the present invention can be prepared by a synthetic method known to a person skilled in the art. For example, they can be prepared according to the following reaction schemes 1 to 3.

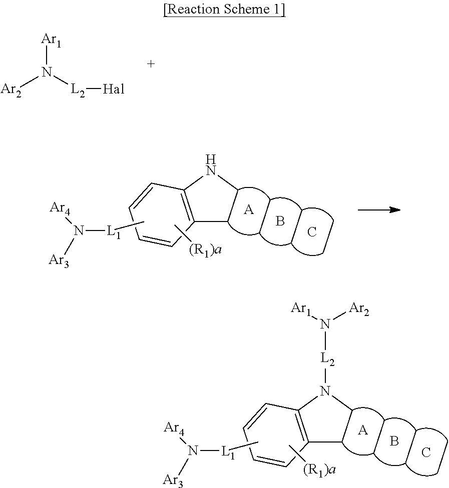

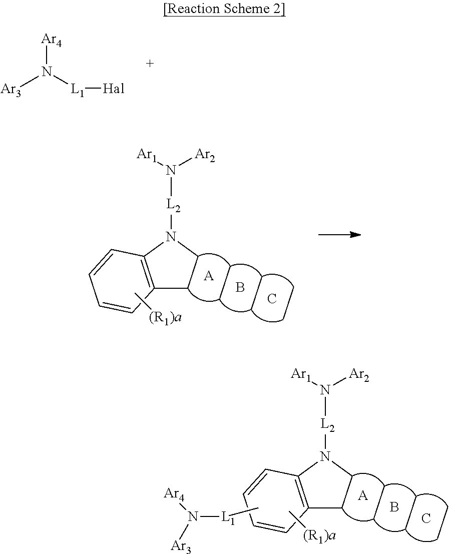

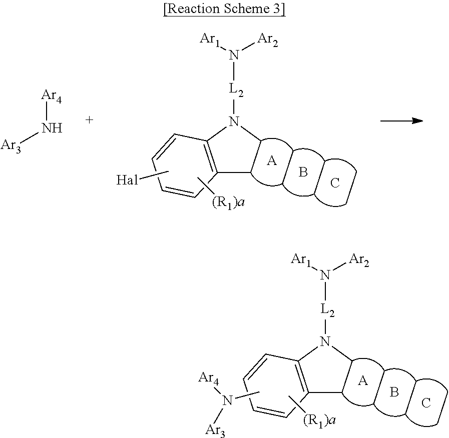

wherein L₁, L₂, Ar₁ to Ar₄, R₁, a, ring A, ring B, ring C are as defined in formula 1 above, and Hal represents a halogen.

In another embodiment of the present invention provides an organic electroluminescent material comprising the organic electroluminescent compound of formula 1, and an organic electroluminescent device comprising the material.

Said organic electroluminescent device comprises a first electrode; a second electrode; and at least one organic layer between said first and second electrodes. Said organic layer comprises at least one organic electroluminescent compound of formula 1 according to the present invention.

One of the first and second electrodes is an anode, and the other is a cathode. The organic layer comprises a light-emitting layer, and at least one layer selected from the group consisting of a hole injection layer, a hole transport layer, an electron transport layer, an electron injection layer, an interlayer, a hole blocking layer, and a electron blocking layer.

The compound represented by formula 1 can be comprised in at least one of the light-emitting layer and the hole transport layer. Where used in the hole transport layer, the compound represented by formula 1 can be comprised as a hole transport material. Where used in the light-emitting layer, the compound represented by formula 1 can be comprised as a host material; preferably, the light-emitting layer can further comprise at least one dopant; and if needed, a compound other than the compound represented by formula 1 can be comprised additionally as a second host material.

The dopant is preferably at least one phosphorescent dopant. The phosphorescent dopant material applied to the electroluminescent device according to the present invention is not limited, but may be preferably selected from metallated complex compounds of iridium, osmium, copper and platinum, more preferably selected from ortho-metallated complex compounds of iridium, osmium, copper and platinum, and even more preferably ortho-metallated iridium complex compounds.

The phosphorescent dopants may be preferably selected from compounds represented by the following formulae 5 to 7.

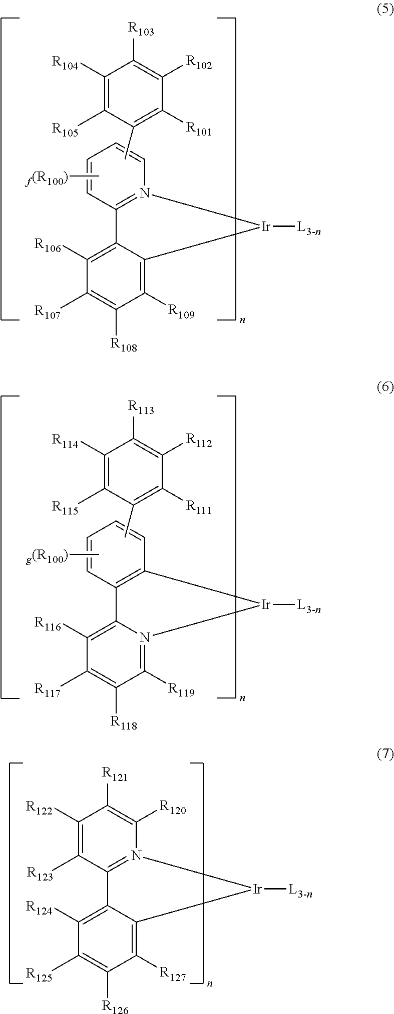

wherein L is selected from the following structures:

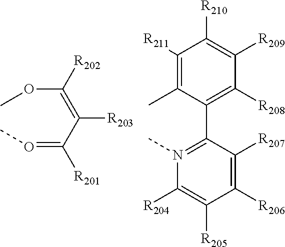

R₁₀₀ represents hydrogen, a substituted or unsubstituted (C1-C30)alkyl group, or a substituted or unsubstituted (C3-C30)cycloalkyl group;

R₁₀₁ to R₁₀₉, and R₁₁₁ to R₁₂₃ each independently represent hydrogen, deuterium, a halogen, a (C1-C30)alkyl group unsubstituted or substituted with halogen(s), a substituted or unsubstituted (C3-C30)cycloalkyl group, a cyano group, or a substituted or unsubstituted (C1-C30)alkoxy group; adjacent substituents of R₁₂₀ to R₁₂₃ may be linked to each other to form a fused ring, e.g. a substituted or unsubstituted quinoline;

R₁₂₄ to R₁₂₇ each independently represent hydrogen, deuterium, a halogen, a substituted or unsubstituted (C1-C30)alkyl group, or a substituted or unsubstituted (C6-C30)aryl group; where R₁₂₄ to R₁₂₇ are aryl groups, adjacent substituents may be linked to each other to form a fused ring, e.g. a substituted or unsubstituted fluorene;

R₂₀₁ to R₂₁₁ each independently represent hydrogen, deuterium, a halogen, a (C1-C30)alkyl group unsubstituted or substituted with halogen(s), a substituted or unsubstituted (C3-C30)cycloalkyl group or a substituted or unsubstituted (C6-C30)aryl group;

f and g each independently represent an integer of 1 to 3; where f or g is an integer of 2 or more, each of R₁₀₀ may be the same or different; and

n is an integer of 0 to 3.

Specifically, the phosphorescent dopant materials include the following:

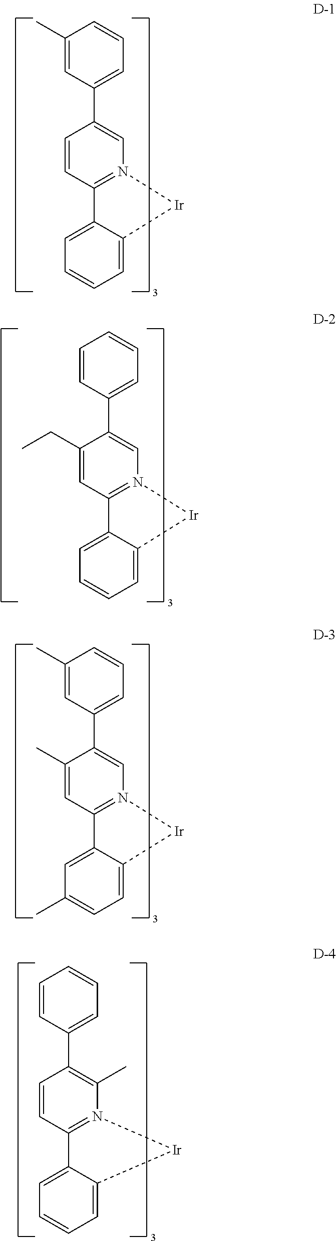

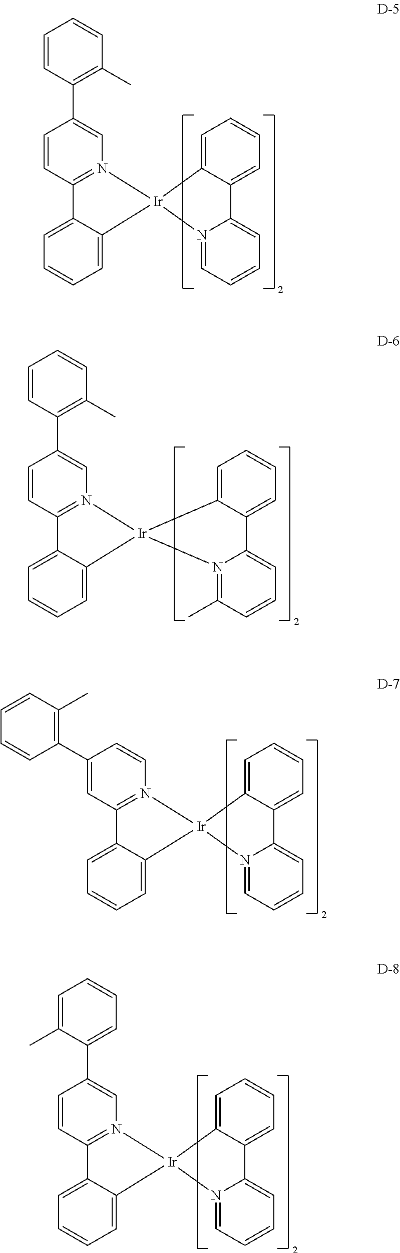

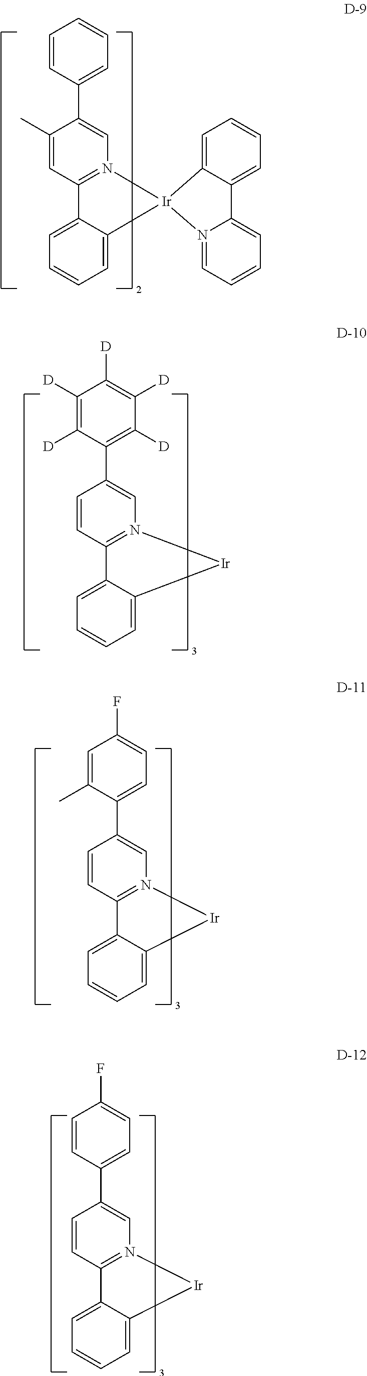

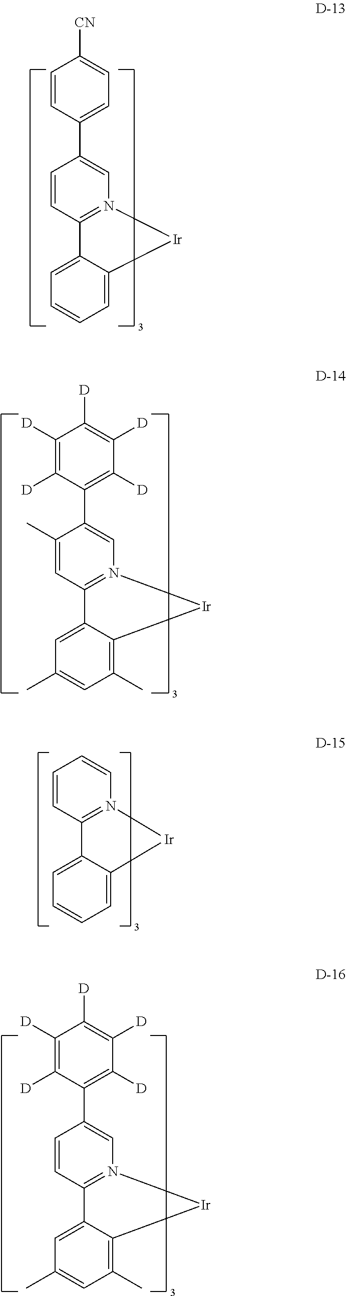

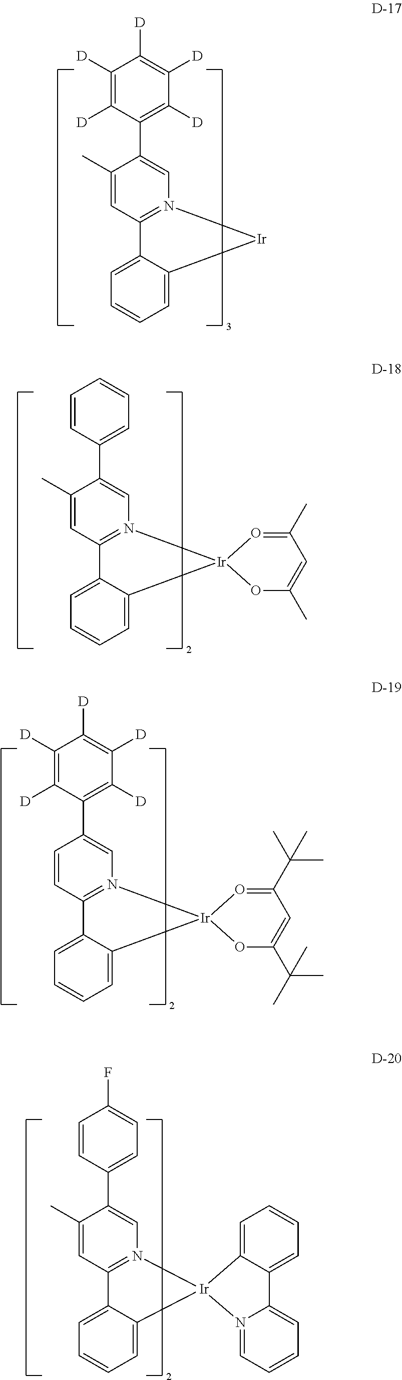

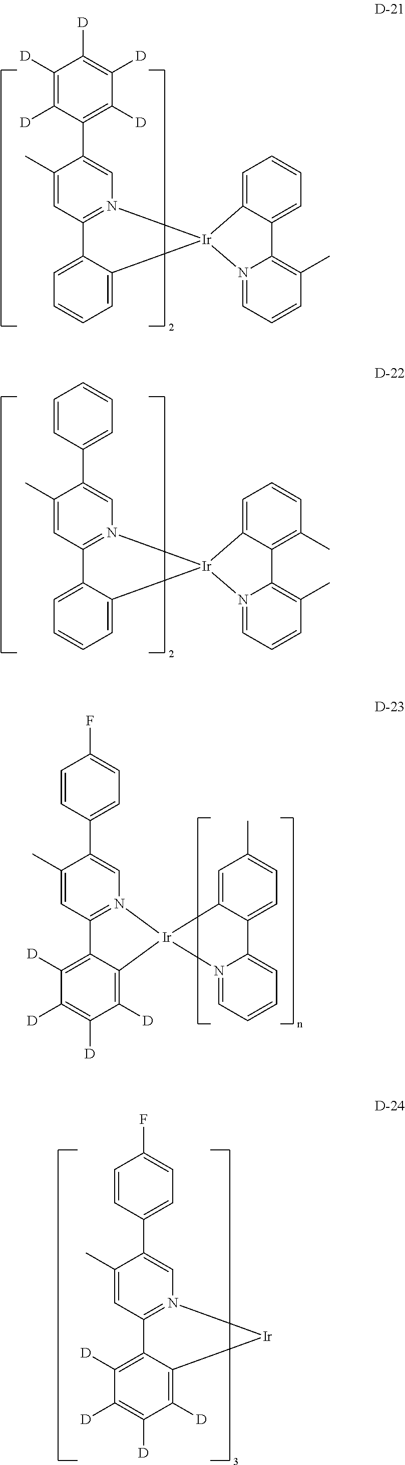

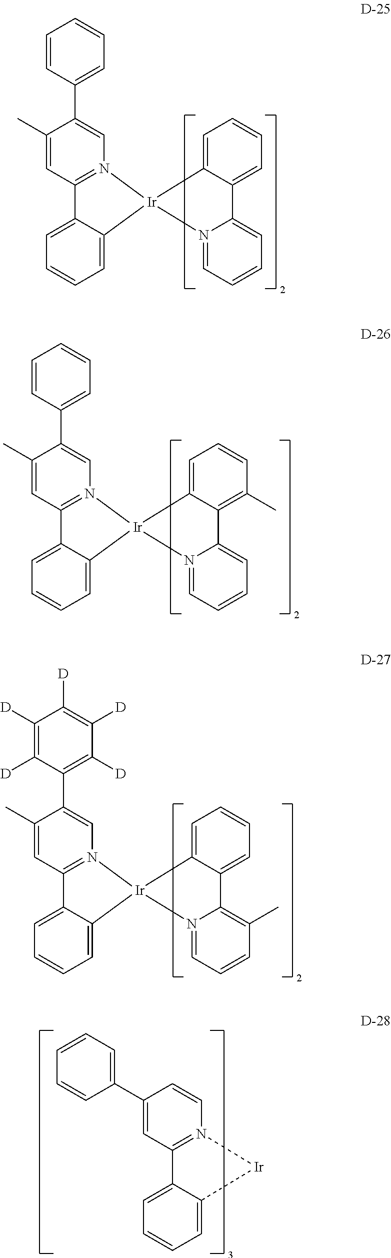

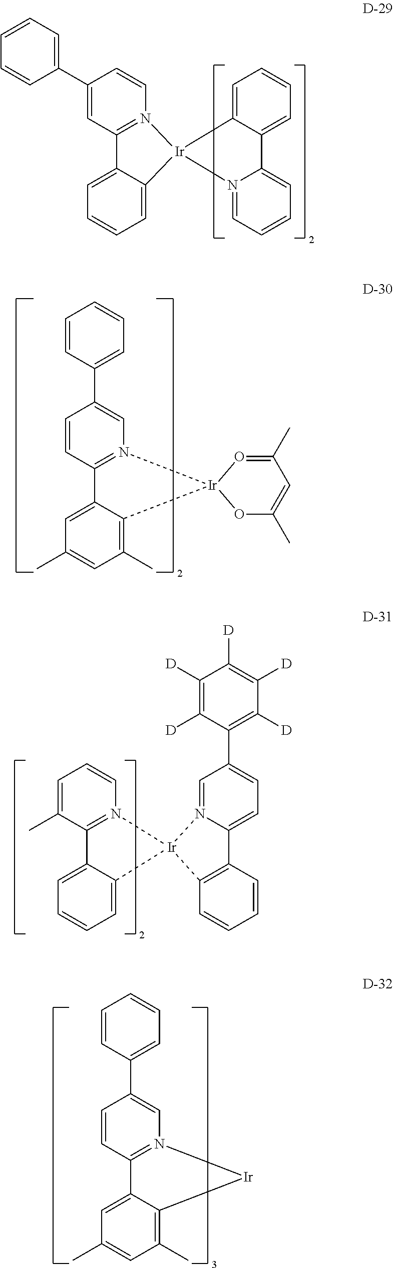

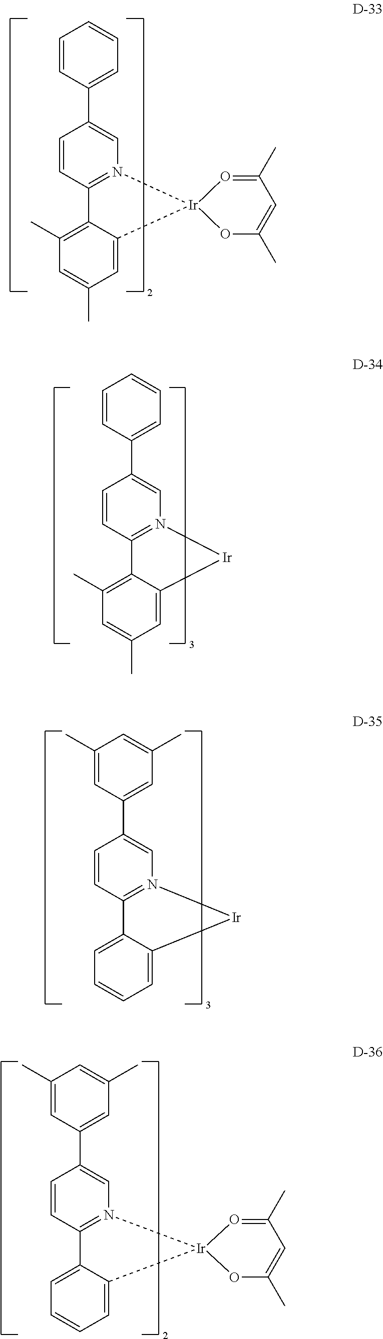

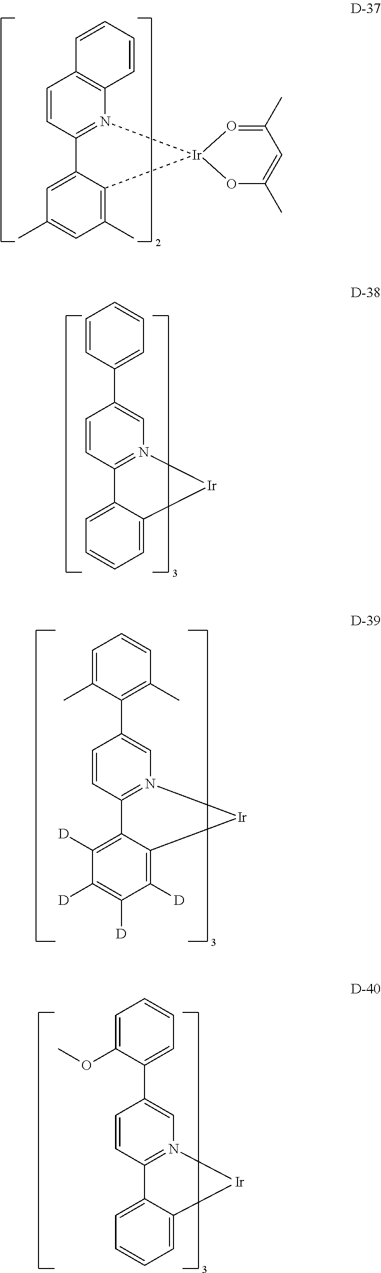

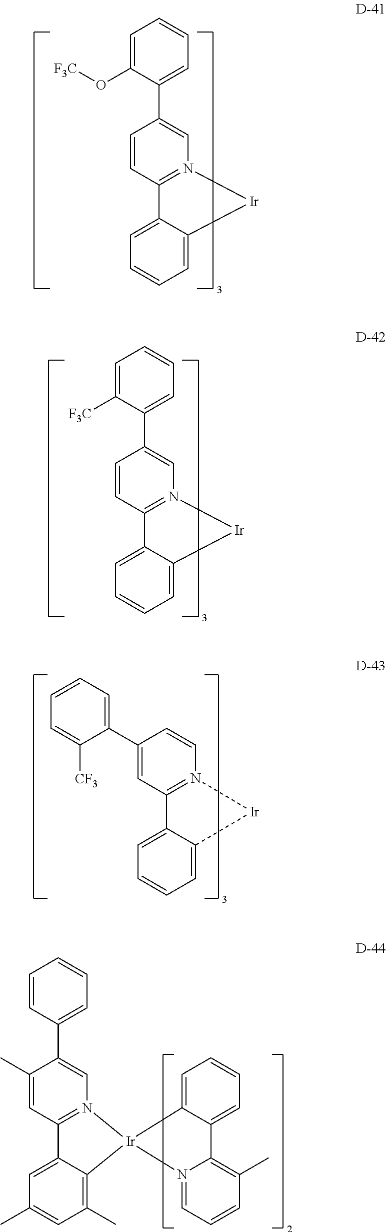

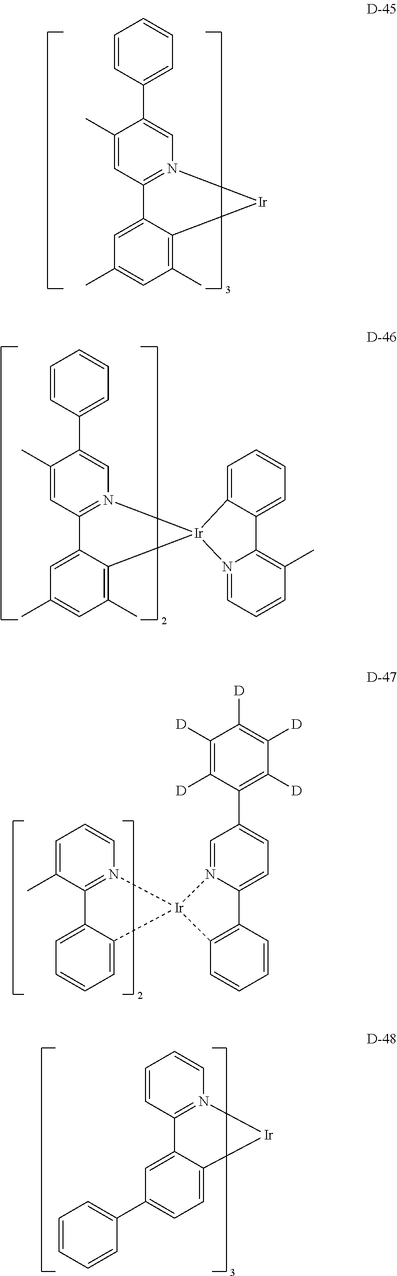

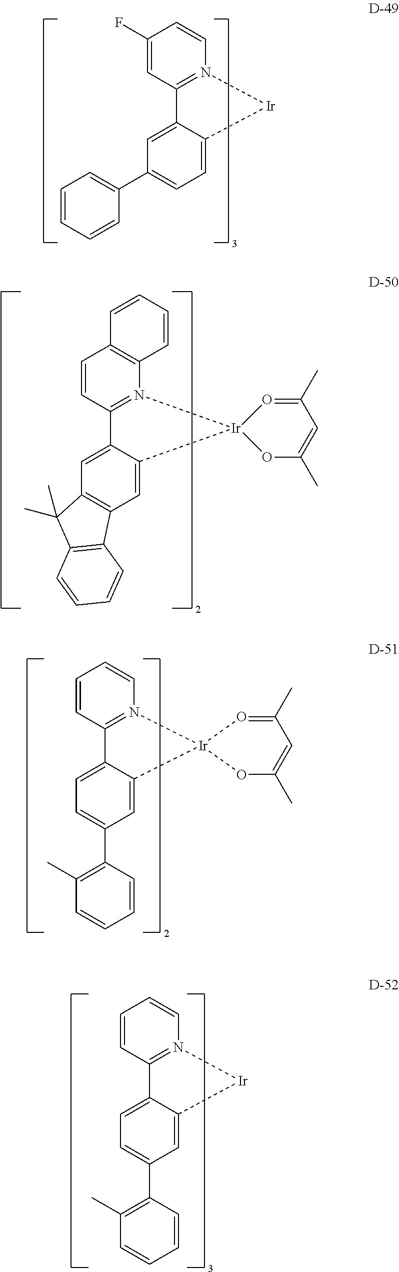

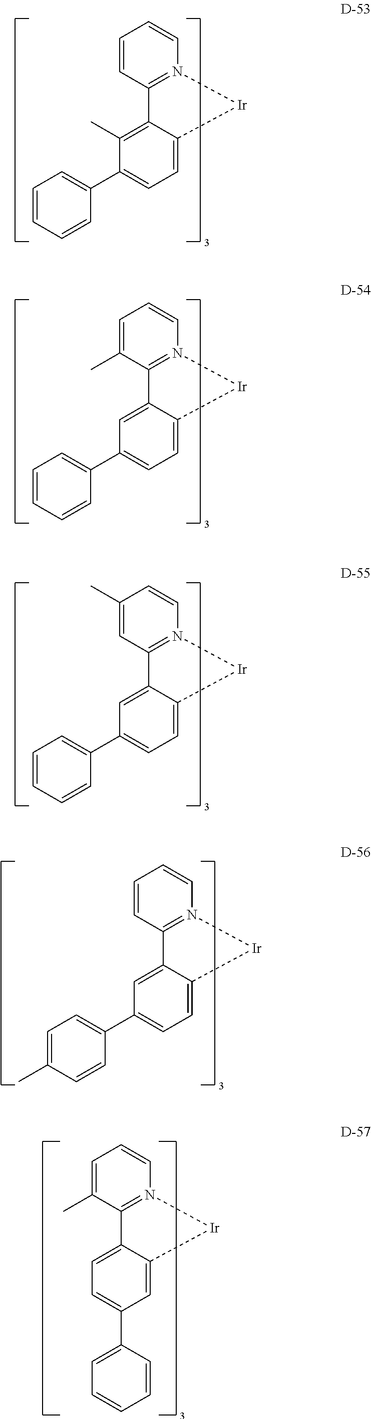

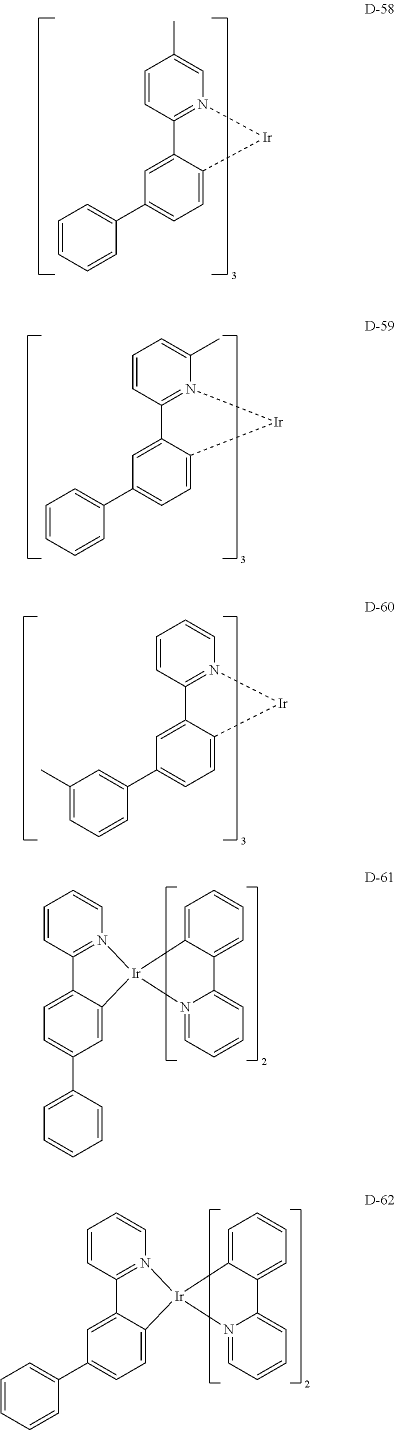

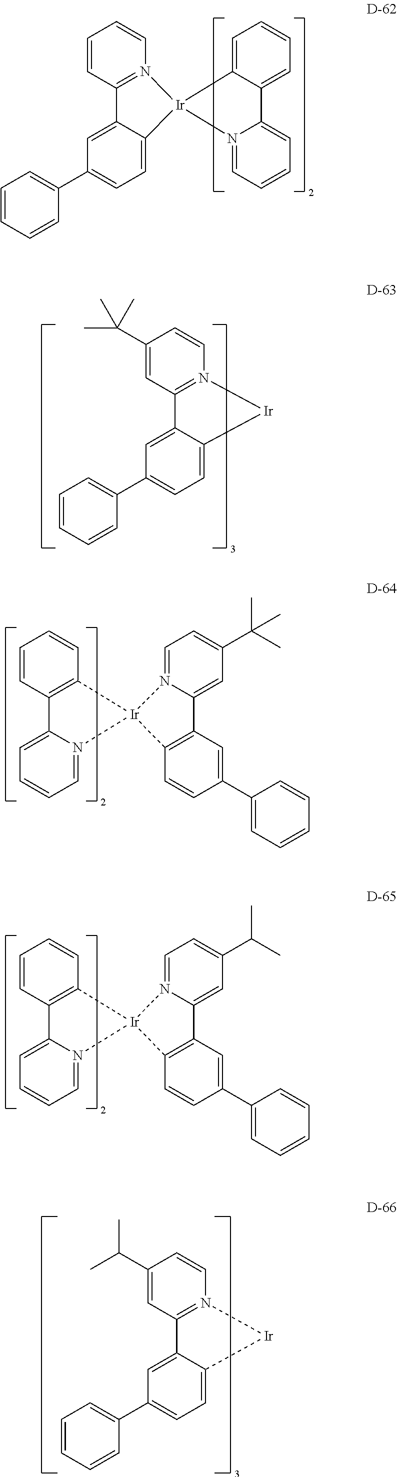

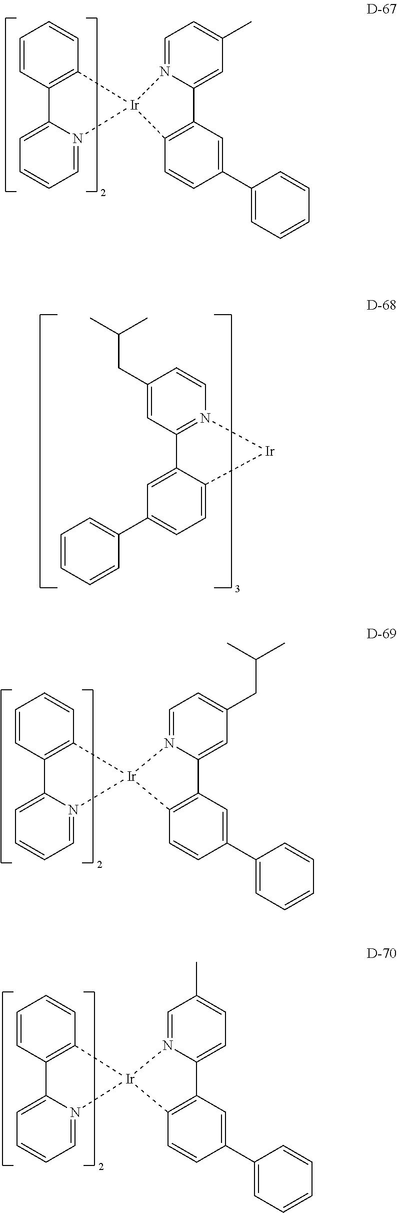

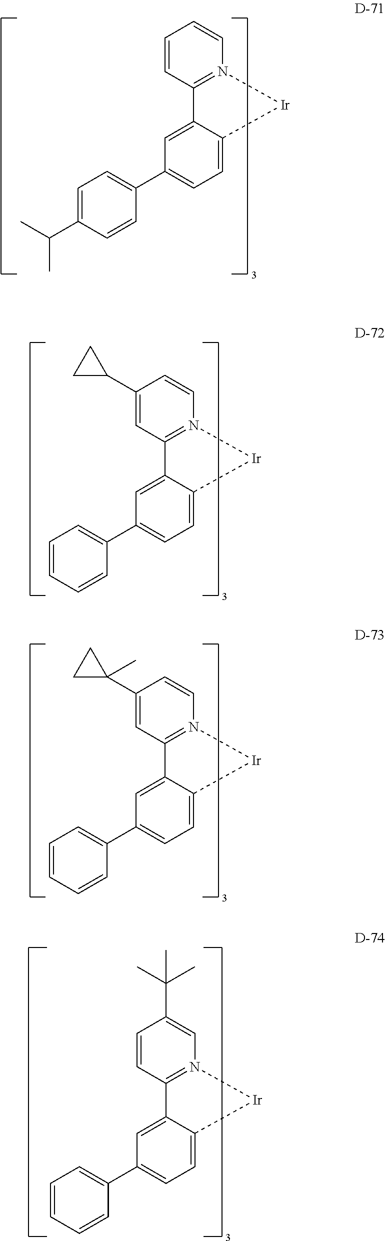

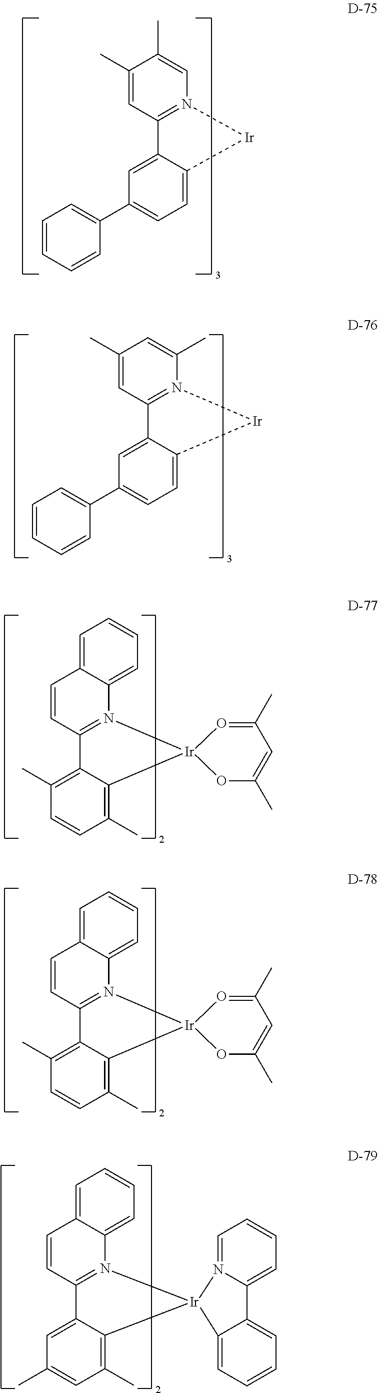

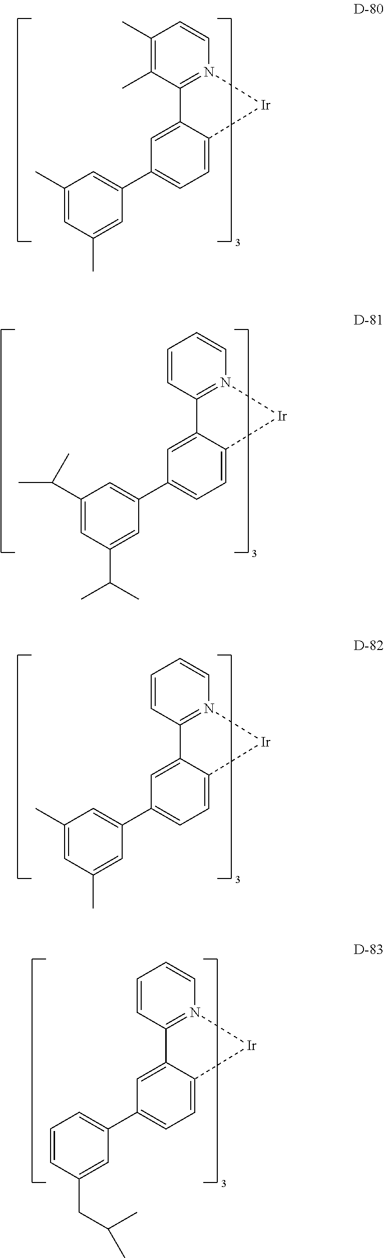

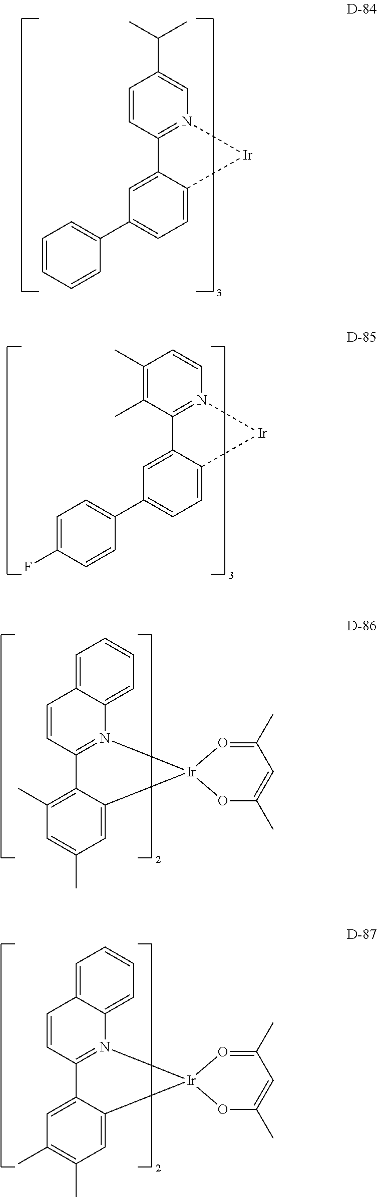

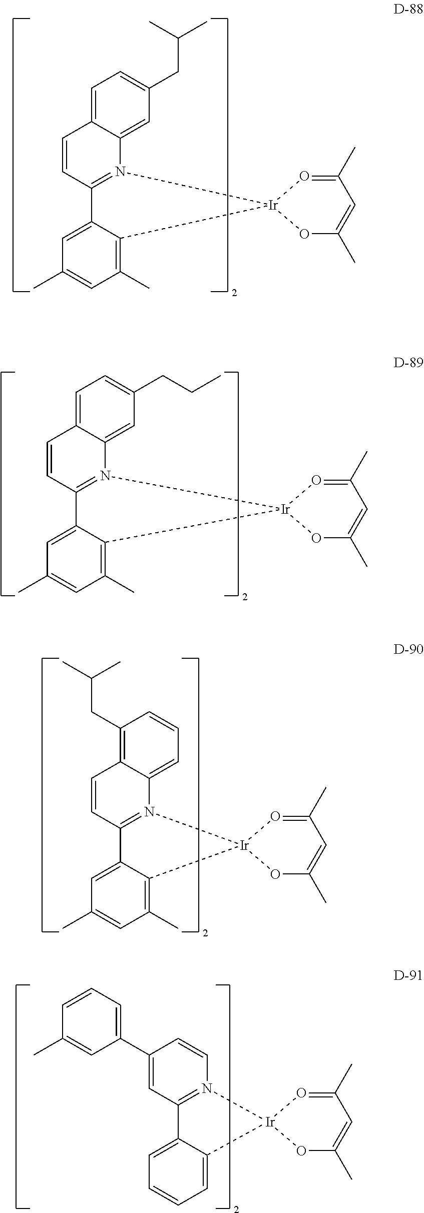

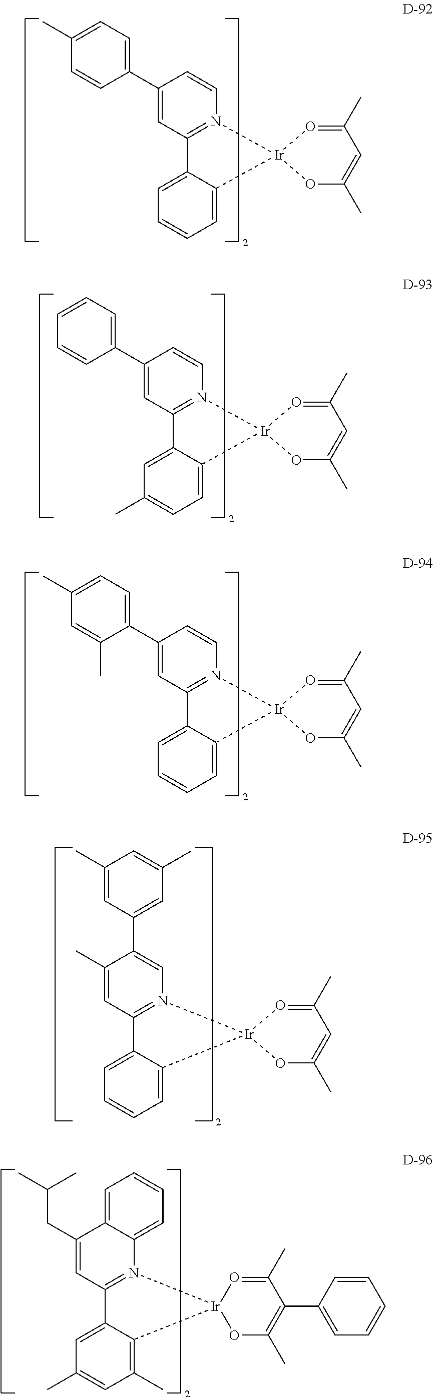

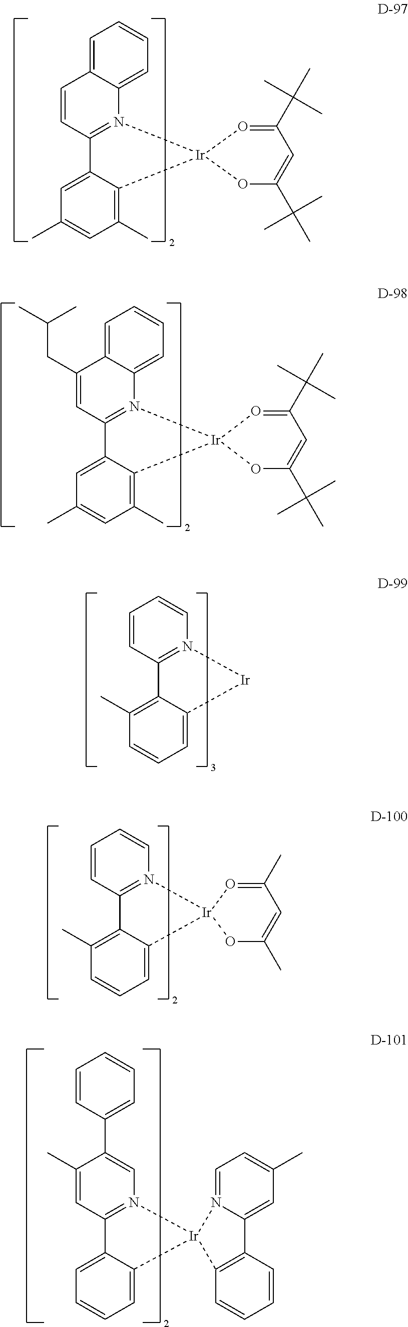

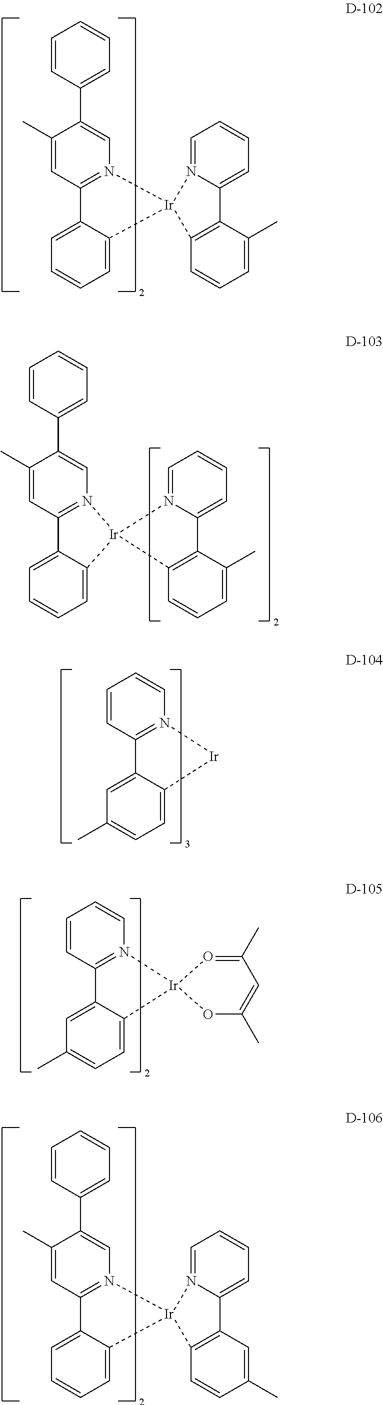

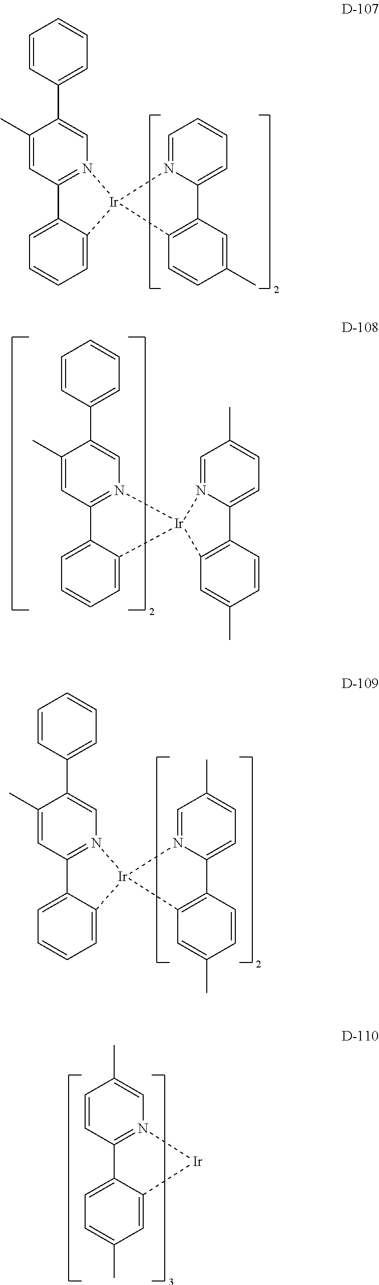

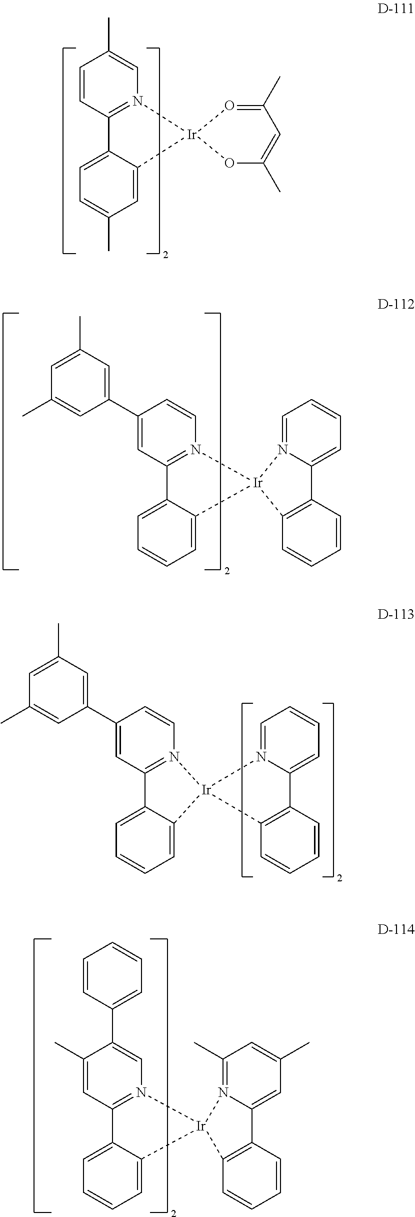

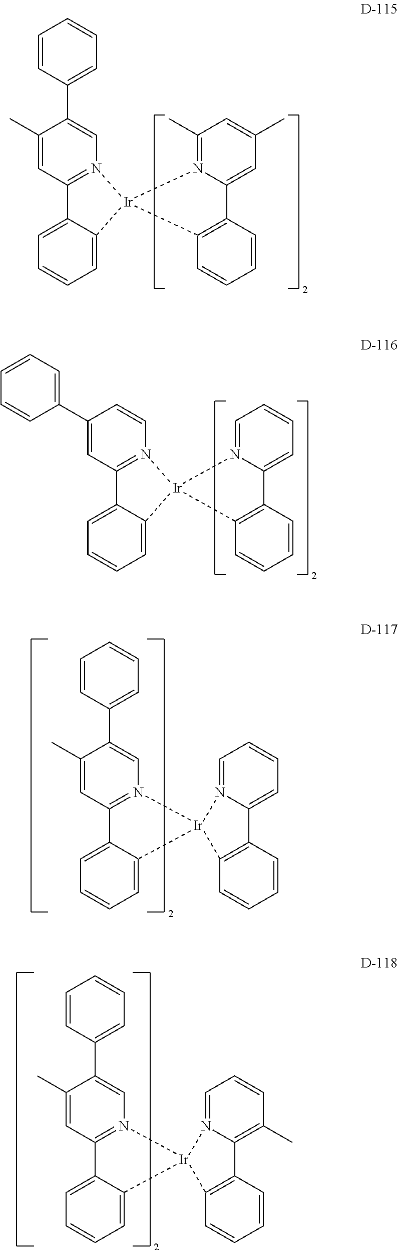

In another embodiment of the present invention provides a composition used for producing an organic electroluminescent device. The composition comprises first host material, and if needed, second host material, and the compound according to the present invention is comprised in the first host material. The ratio of the first host material to the second host material is in the range of 1:99 to 99:1.

The second host material may be selected from the phosphorescent host represented by formula 8 to 14 below.

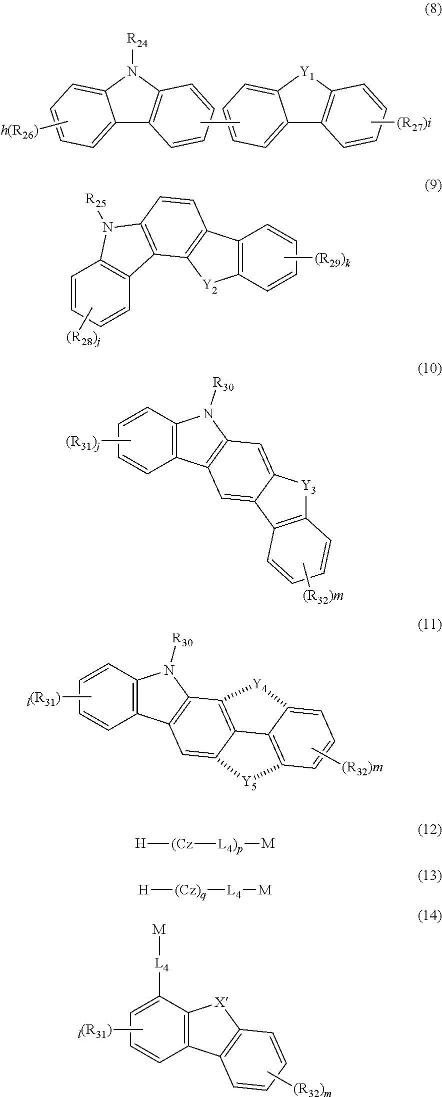

wherein Cz represents the following structure;

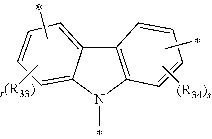

X′ represents —O— or —S—;

R₂₄, R₂₅, and R₃₀ each independently represent a substituted of unsubstituted (C6-C30)aryl group, or a substituted or unsubstituted 5- to 30-membered heteroaryl group;

R₂₆ to R₂₉, and R₃₁ to R₃₄ each independently represent hydrogen, deuterium, a halogen, a substituted or unsubstituted (C1-C30)alkyl group, a substituted of unsubstituted (C6-C30)aryl group, a substituted or unsubstituted 5- to 30-membered heteroaryl group, or R₃₅R₃₆R₃₇Si—;

R₃₅ to R₃₇ each independently represent a substituted or unsubstituted (C1-C30)alkyl group, or a substituted or unsubstituted (C6-C30)aryl group;

L₄ represents a single bond, a substituted or unsubstituted (C6-C30)arylene group, or a substituted or unsubstituted 5- to 30-membered heteroarylene group;

M represents a substituted or unsubstituted (C6-C30)aryl group, or a substituted or unsubstituted 5- to 30-membered heteroaryl group;

Y₁ to Y₅ each independently represent —O—, —S—, —N(R₄₁)— or —C(R₄₂)(R₄₃)—, provided that Y₄ and Y₅ do not simultaneously exist;

R₄₁ to R₄₃ each independently represent a substituted or unsubstituted (C1-C30)alkyl group, a substituted or unsubstituted (C6-C30)aryl group, or a substituted or unsubstituted 5- to 30-membered heteroaryl group, and R₄₂ and R₄₃ may be same or different;

p and q each independently represent an integer of 1 to 3;

h, i, j, k, l, m, r, and s each independently represent an integer of 1 to 4; and

where h, i, j, k, l, m, p, q, r, or s is an integer of 2 or more, each of R₂₆, each of R₂₇, each of R₂₈, each of R₂₉, each of R₃₁, each of R₃₂, each of (Cz-L₄), each of (Cz), each of R₃₃, or each of R₃₄ may be same or different.

Specifically, the second host materials include the following:

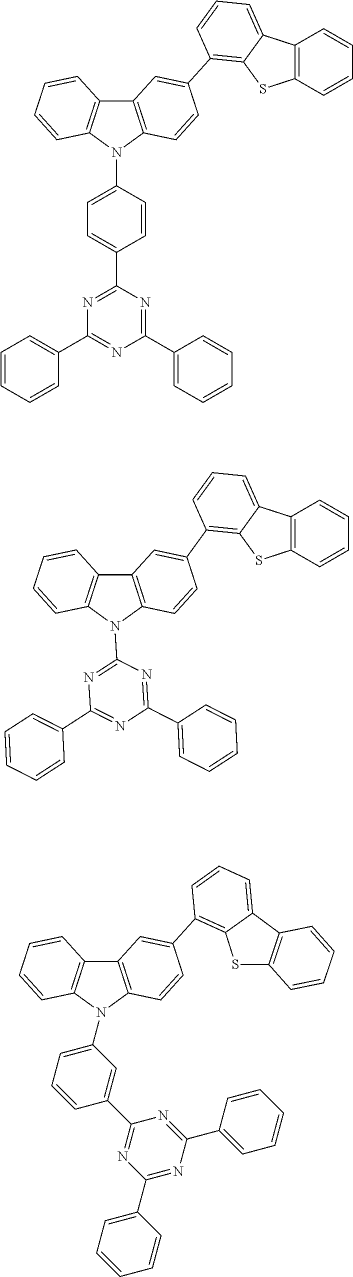 [SMILES: c1ccc(-c2nc(-c3ccccc3)nc(-c3ccc(-n4c5ccccc5c5cc(-c6cccc7c6sc6ccccc67)ccc54)cc3)n2)cc1 ; c1ccc(-c2nc(-c3ccccc3)nc(-n3c4ccccc4c4cc(-c5cccc6c5sc5ccccc56)ccc43)n2)cc1 ; c1ccc(-c2nc(-c3ccccc3)nc(-c3cccc(-n4c5ccccc5c5cc(-c6cccc7c6sc6ccccc67)ccc54)c3)n2)cc1]

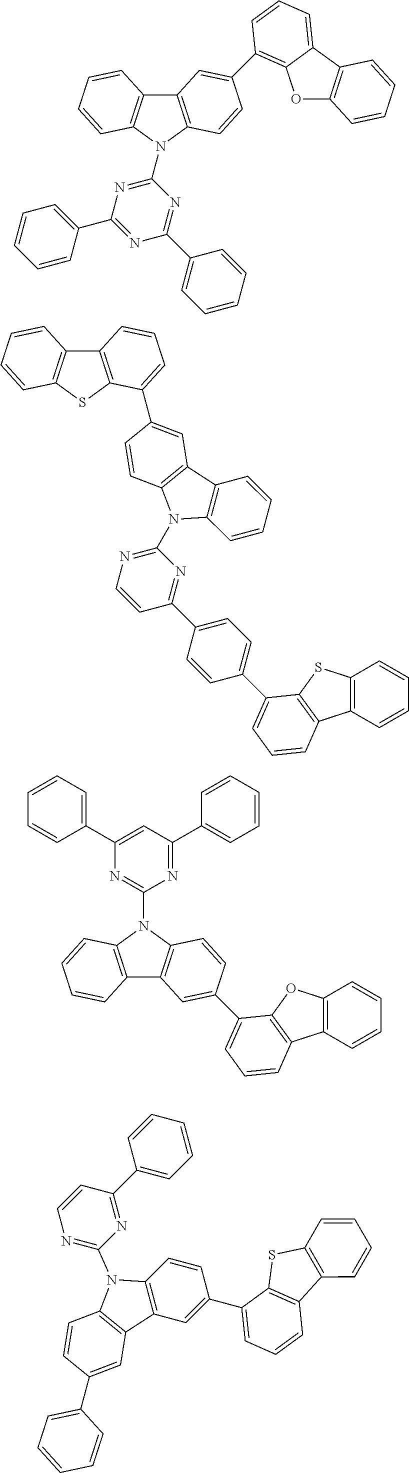

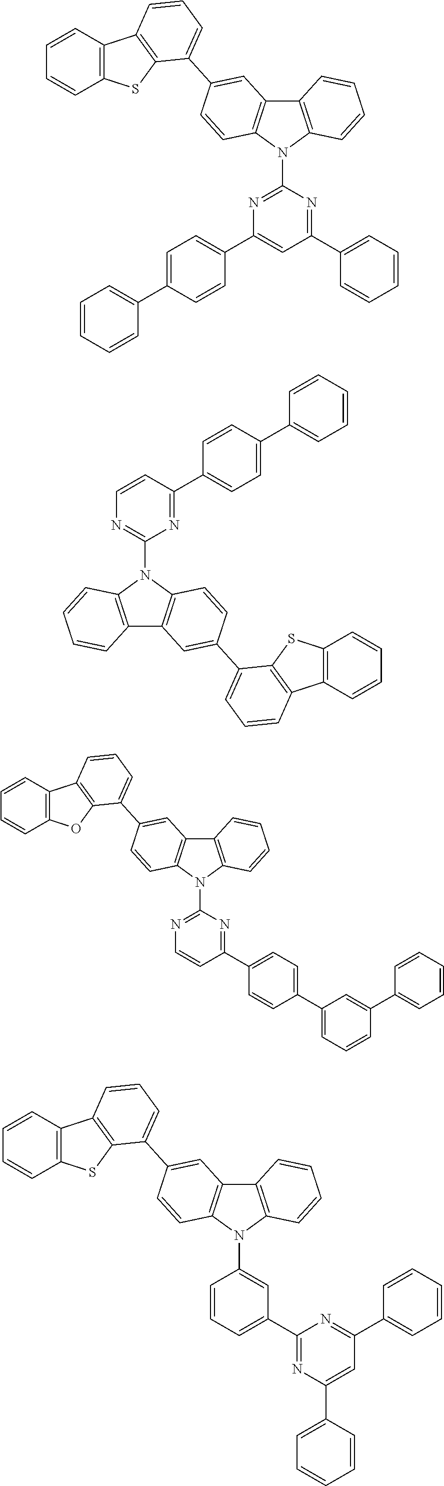 [SMILES: c1ccc(-c2ccc(-c3cc(-c4ccccc4)nc(-n4c5ccccc5c5cc(-c6cccc7c6sc6ccccc67)ccc54)n3)cc2)cc1 ; c1ccc(-c2ccc(-c3ccnc(-n4c5ccccc5c5cc(-c6cccc7c6sc6ccccc67)ccc54)n3)cc2)cc1 ; c1ccc(-c2cccc(-c3ccc(-c4ccnc(-n5c6ccccc6c6cc(-c7cccc8c7oc7ccccc78)ccc65)n4)cc3)c2)cc1 ; c1ccc(-c2cc(-c3ccccc3)nc(-c3cccc(-n4c5ccccc5c5cc(-c6cccc7c6sc6ccccc67)ccc54)c3)n2)cc1]

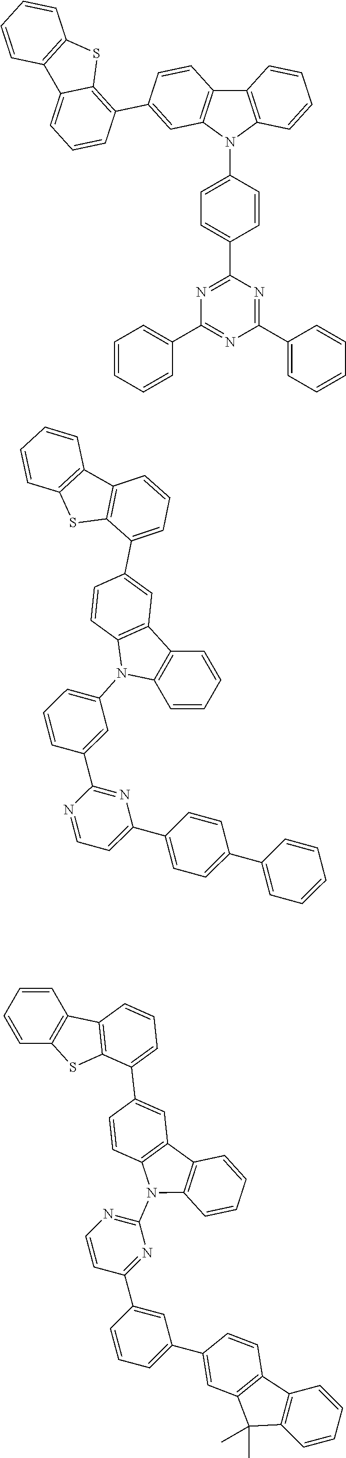 [SMILES: c1ccc(-c2nc(-c3ccccc3)nc(-c3ccc(-n4c5ccccc5c5ccc(-c6cccc7c6sc6ccccc67)cc54)cc3)n2)cc1 ; c1ccc(-c2ccc(-c3ccnc(-c4cccc(-n5c6ccccc6c6cc(-c7cccc8c7sc7ccccc78)ccc65)c4)n3)cc2)cc1 ; CC1(C)c2ccccc2-c2ccc(-c3cccc(-c4ccnc(-n5c6ccccc6c6cc(-c7cccc8c7sc7ccccc78)ccc65)n4)c3)cc21]

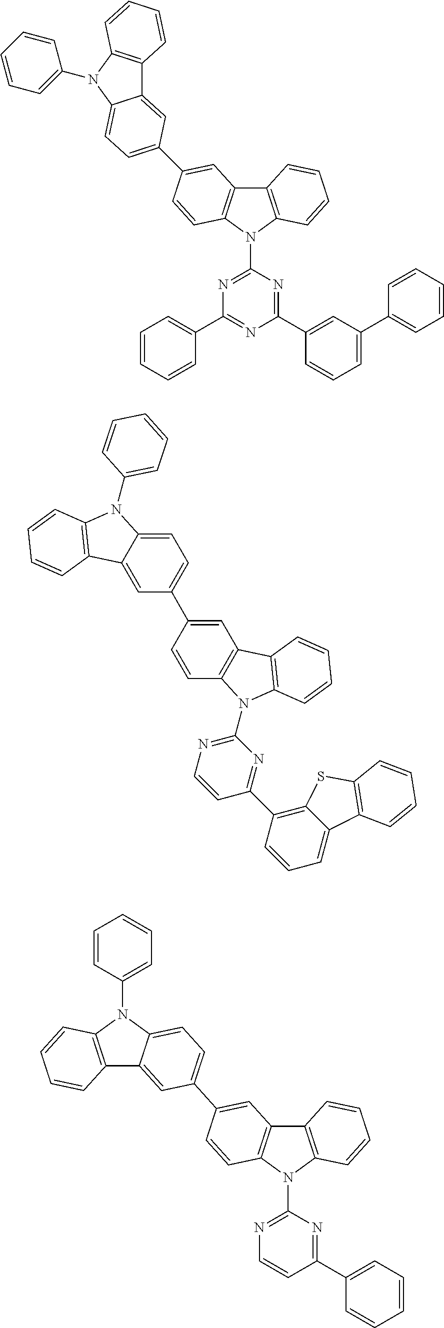

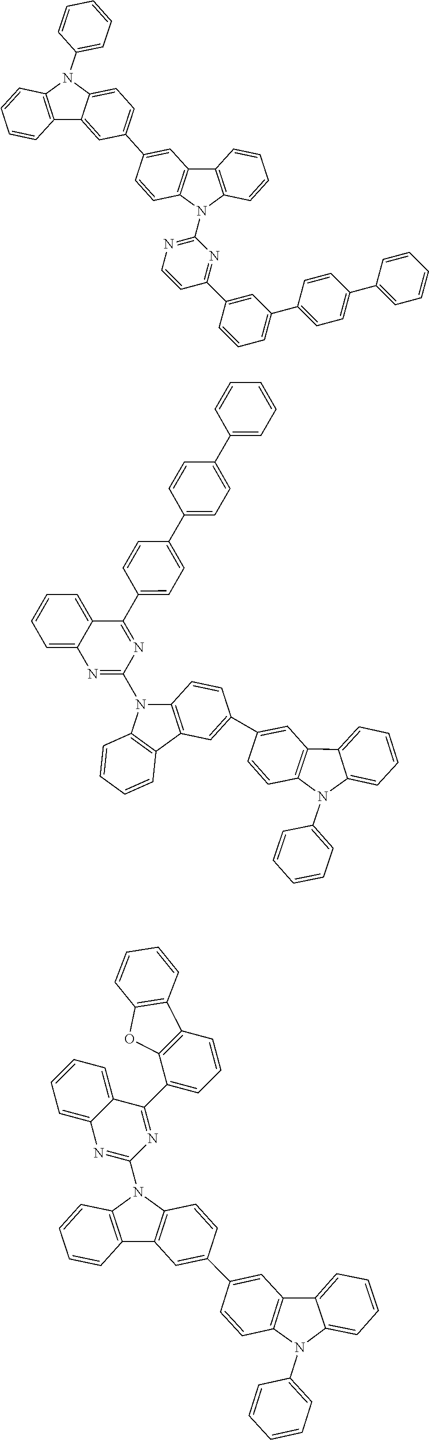

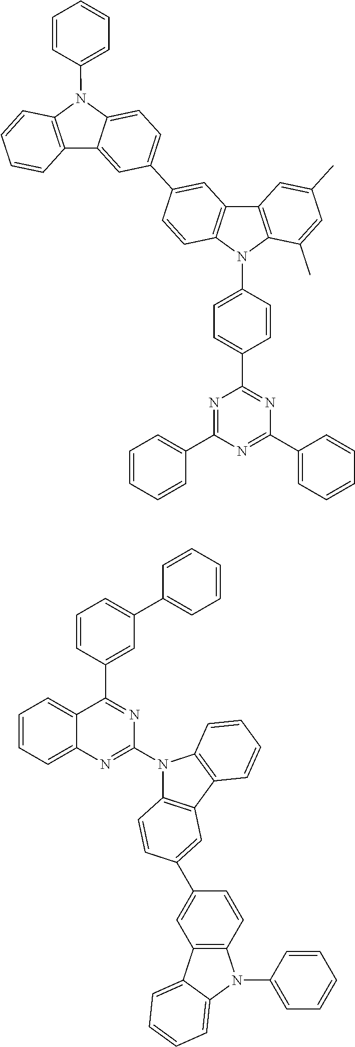 [SMILES: Cc1cc(C)c2c(c1)c1cc(-c3ccc4c(c3)c3ccccc3n4-c3ccccc3)ccc1n2-c1ccc(-c2nc(-c3ccccc3)nc(-c3ccccc3)n2)cc1 ; c1ccc(-c2cccc(-c3nc(-n4c5ccccc5c5cc(-c6ccc7c(c6)c6ccccc6n7-c6ccccc6)ccc54)nc4ccccc34)c2)cc1]

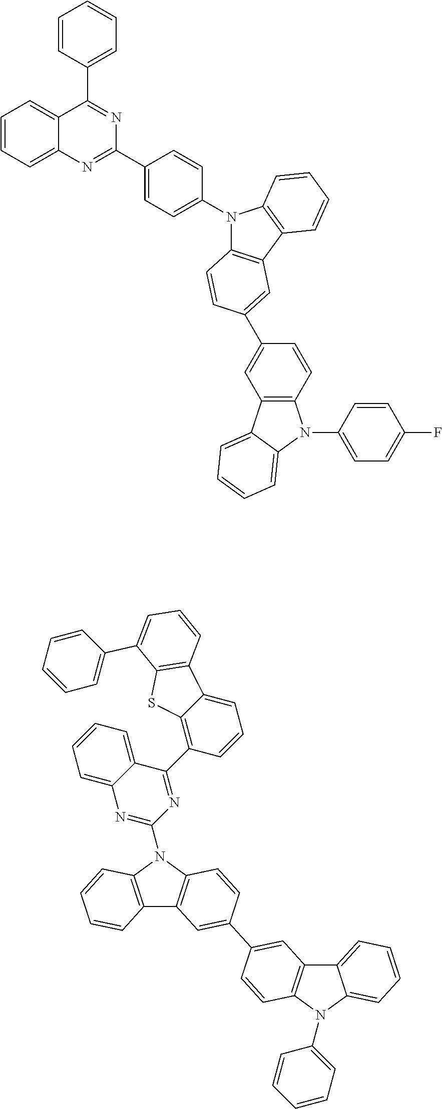 [SMILES: Fc1ccc(-n2c3ccccc3c3cc(-c4ccc5c(c4)c4ccccc4n5-c4ccc(-c5nc(-c6ccccc6)c6ccccc6n5)cc4)ccc32)cc1 ; c1ccc(-c2cccc3c2sc2c(-c4nc(-n5c6ccccc6c6cc(-c7ccc8c(c7)c7ccccc7n8-c7ccccc7)ccc65)nc5ccccc45)cccc23)cc1]

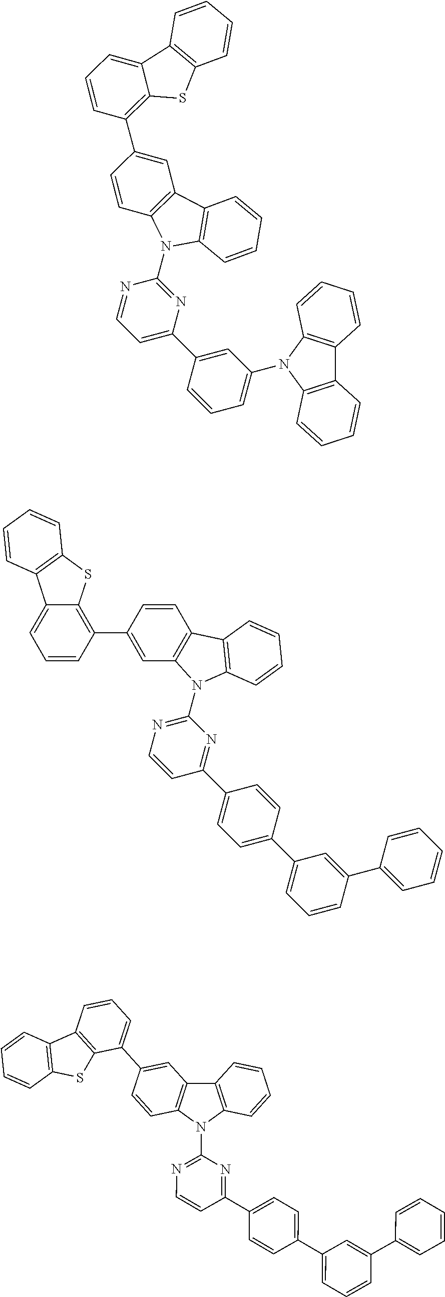 [SMILES: c1cc(-c2ccnc(-n3c4ccccc4c4cc(-c5cccc6c5sc5ccccc56)ccc43)n2)cc(-n2c3ccccc3c3ccccc32)c1 ; c1ccc(-c2cccc(-c3ccc(-c4ccnc(-n5c6ccccc6c6ccc(-c7cccc8c7sc7ccccc78)cc65)n4)cc3)c2)cc1 ; c1ccc(-c2cccc(-c3ccc(-c4ccnc(-n5c6ccccc6c6cc(-c7cccc8c7sc7ccccc78)ccc65)n4)cc3)c2)cc1]

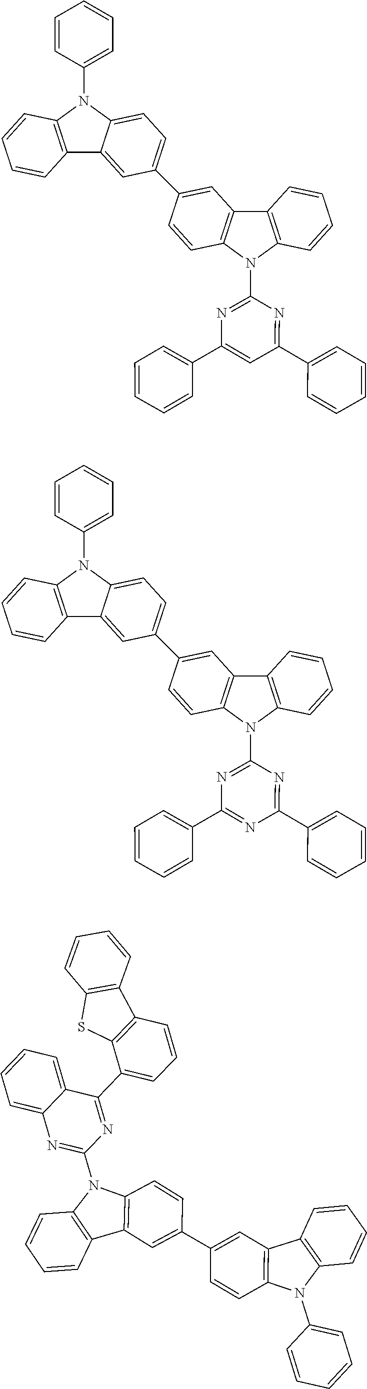 [SMILES: c1ccc(-c2cc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-n2c3ccccc3c3cc(-c4ccc5c(c4)c4ccccc4n5-c4nc(-c5cccc6c5sc5ccccc56)c5ccccc5n4)ccc32)cc1]

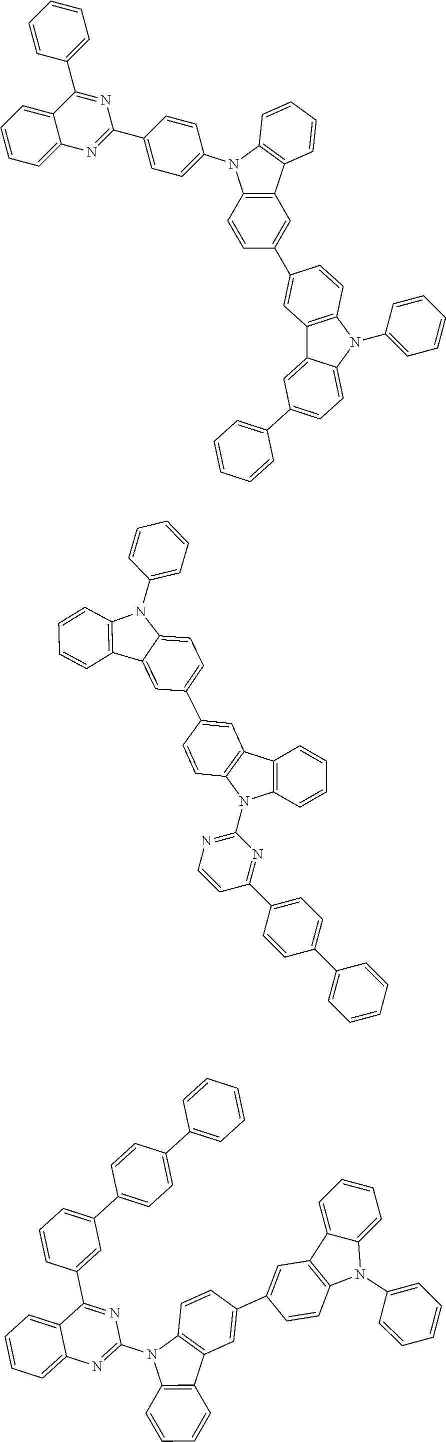 [SMILES: c1ccc(-c2ccc3c(c2)c2cc(-c4ccc5c(c4)c4ccccc4n5-c4ccc(-c5nc(-c6ccccc6)c6ccccc6n5)cc4)ccc2n3-c2ccccc2)cc1 ; c1ccc(-c2ccc(-c3ccnc(-n4c5ccccc5c5cc(-c6ccc7c(c6)c6ccccc6n7-c6ccccc6)ccc54)n3)cc2)cc1 ; c1ccc(-c2ccc(-c3cccc(-c4nc(-n5c6ccccc6c6cc(-c7ccc8c(c7)c7ccccc7n8-c7ccccc7)ccc65)nc5ccccc45)c3)cc2)cc1]

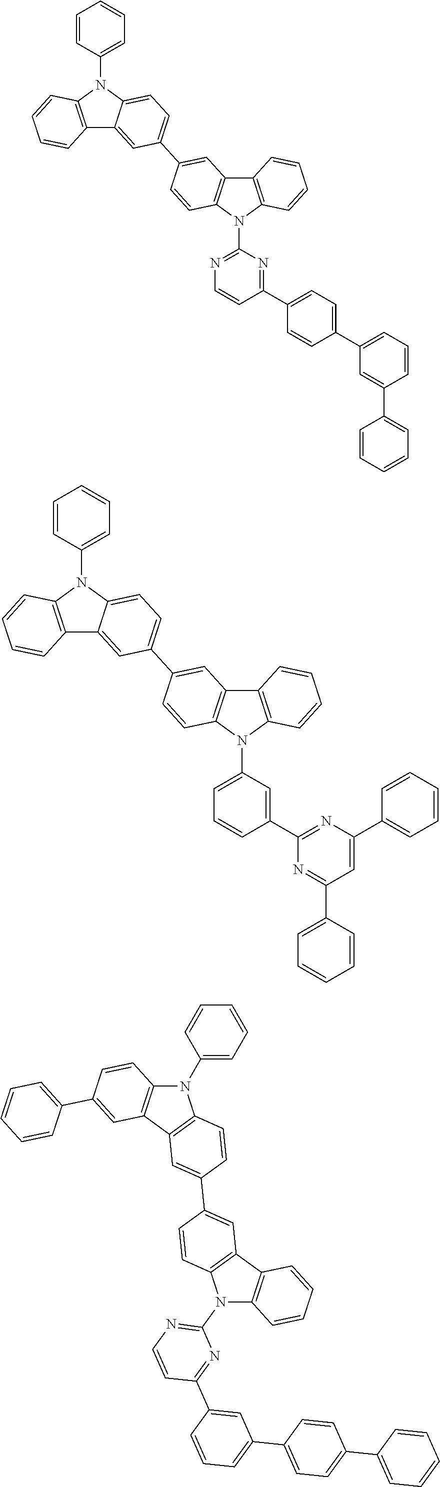 [SMILES: c1ccc(-c2cccc(-c3ccc(-c4ccnc(-n5c6ccccc6c6cc(-c7ccc8c(c7)c7ccccc7n8-c7ccccc7)ccc65)n4)cc3)c2)cc1 ; c1ccc(-c2cc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cccc(-c4ccnc(-n5c6ccccc6c6cc(-c7ccc8c(c7)c7cc(-c9ccccc9)ccc7n8-c7ccccc7)ccc65)n4)c3)cc2)cc1]

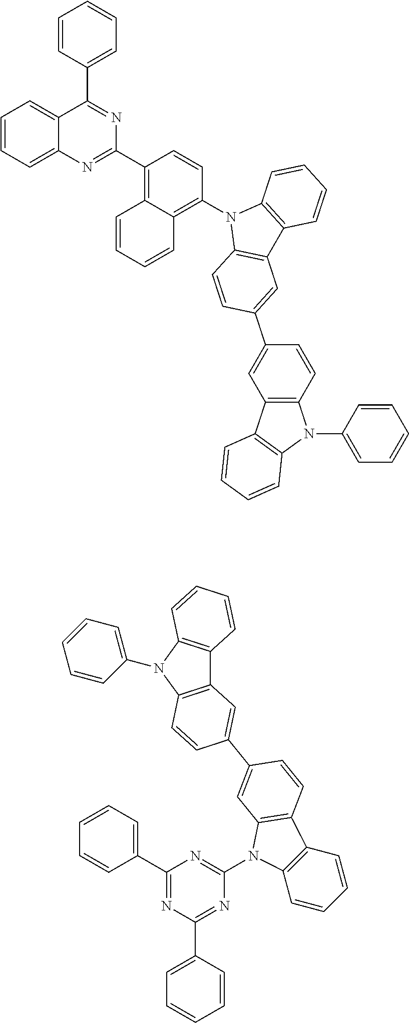 [SMILES: c1ccc(-c2nc(-c3ccc(-n4c5ccccc5c5cc(-c6ccc7c(c6)c6ccccc6n7-c6ccccc6)ccc54)c4ccccc34)nc3ccccc23)cc1 ; c1ccc(-c2nc(-c3ccccc3)nc(-n3c4ccccc4c4ccc(-c5ccc6c(c5)c5ccccc5n6-c5ccccc5)cc43)n2)cc1]

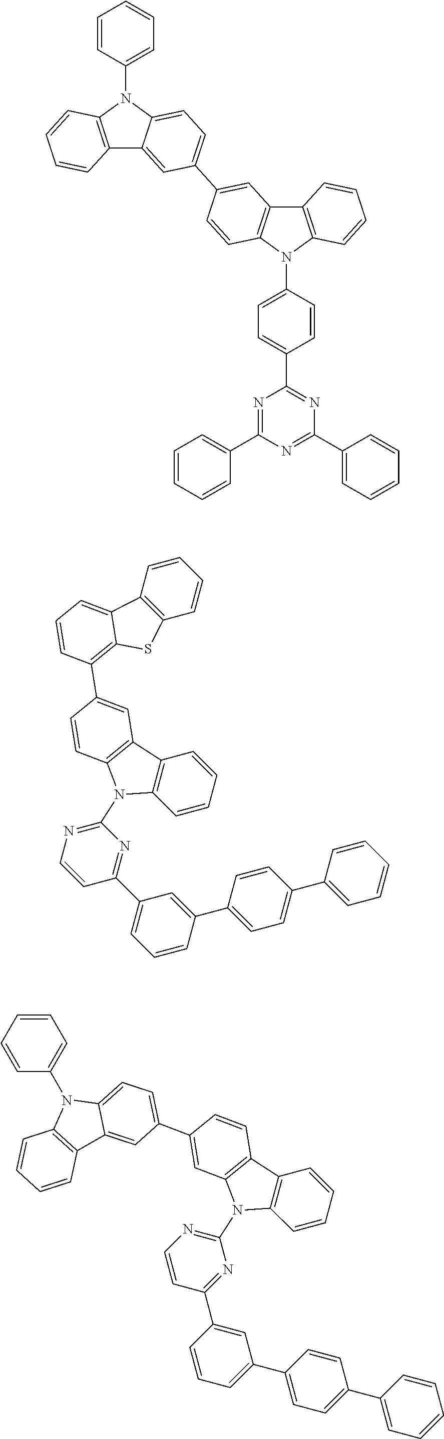 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2ccc(-c3cccc(-c4ccnc(-n5c6ccccc6c6cc(-c7cccc8c7sc7ccccc78)ccc65)n4)c3)cc2)cc1 ; c1ccc(-c2ccc(-c3cccc(-c4ccnc(-n5c6ccccc6c6ccc(-c7ccc8c(c7)c7ccccc7n8-c7ccccc7)cc65)n4)c3)cc2)cc1]

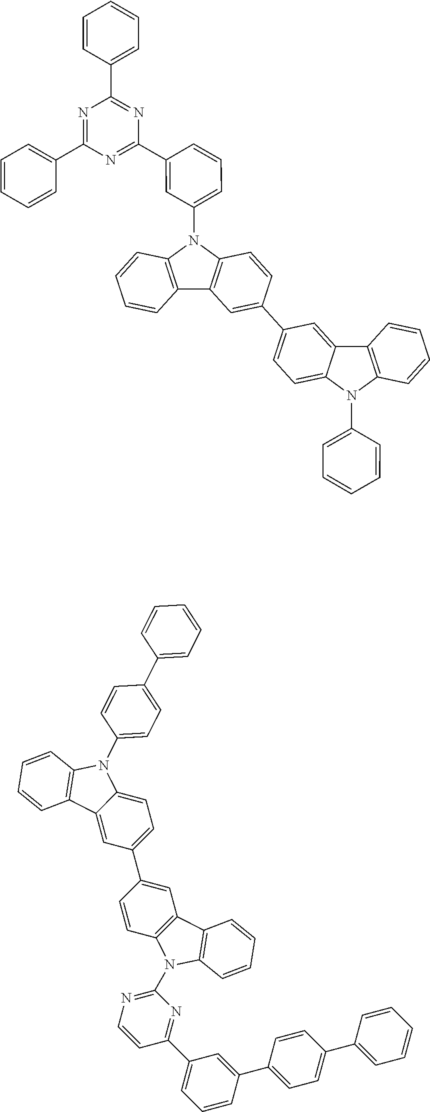 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cccc(-c4ccnc(-n5c6ccccc6c6cc(-c7ccc8c(c7)c7ccccc7n8-c7ccc(-c8ccccc8)cc7)ccc65)n4)c3)cc2)cc1]

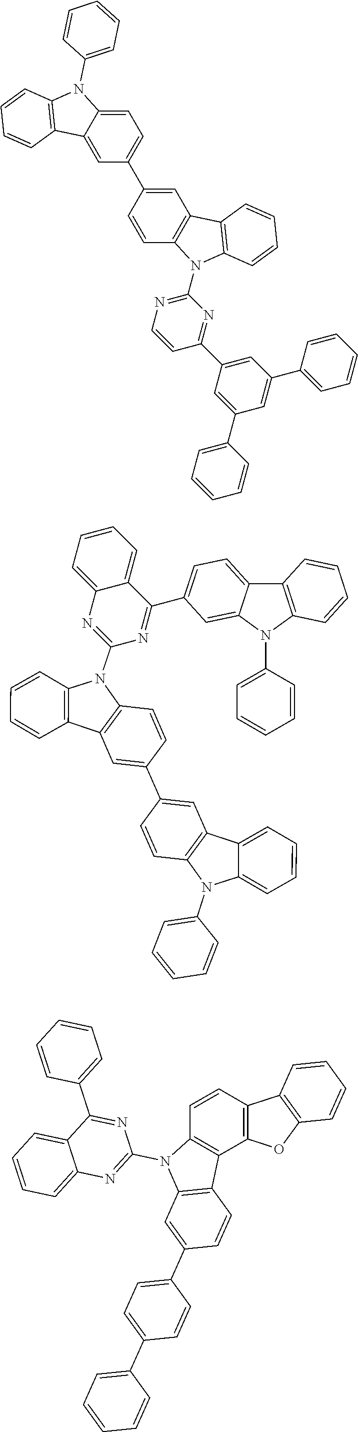 [SMILES: c1ccc(-c2cc(-c3ccccc3)cc(-c3ccnc(-n4c5ccccc5c5cc(-c6ccc7c(c6)c6ccccc6n7-c6ccccc6)ccc54)n3)c2)cc1 ; c1ccc(-n2c3ccccc3c3cc(-c4ccc5c(c4)c4ccccc4n5-c4nc(-c5ccc6c7ccccc7n(-c7ccccc7)c6c5)c5ccccc5n4)ccc32)cc1 ; c1ccc(-c2ccc(-c3ccc4c5c6oc7ccccc7c6ccc5n(-c5nc(-c6ccccc6)c6ccccc6n5)c4c3)cc2)cc1]

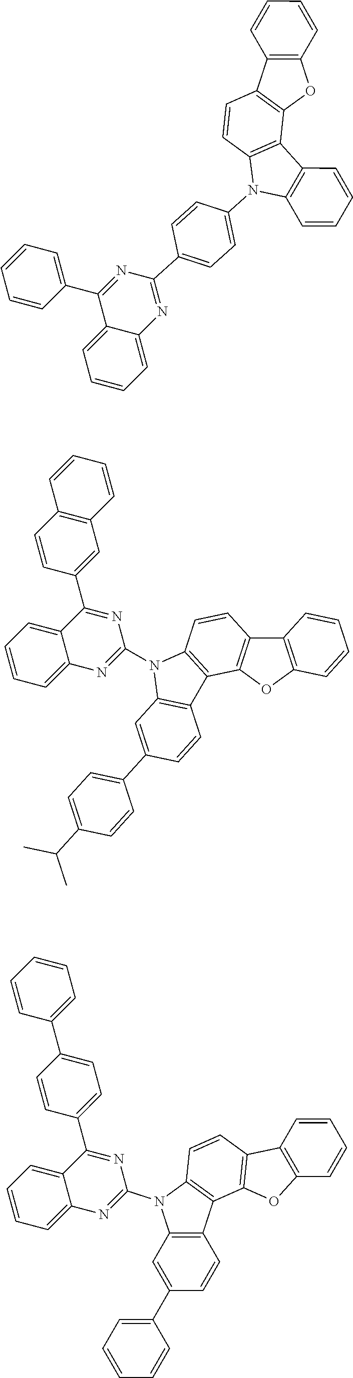 [SMILES: c1ccc(-c2nc(-c3ccc(-n4c5ccccc5c5c6oc7ccccc7c6ccc54)cc3)nc3ccccc23)cc1 ; CC(C)c1ccc(-c2ccc3c4c5oc6ccccc6c5ccc4n(-c4nc(-c5ccc6ccccc6c5)c5ccccc5n4)c3c2)cc1 ; c1ccc(-c2ccc(-c3nc(-n4c5cc(-c6ccccc6)ccc5c5c6oc7ccccc7c6ccc54)nc4ccccc34)cc2)cc1]

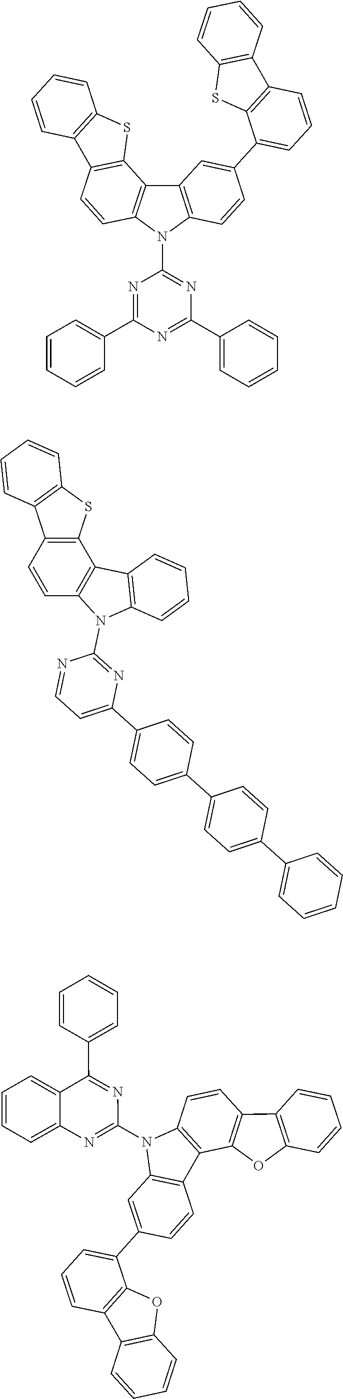 [SMILES: c1ccc(-c2nc(-c3ccccc3)nc(-n3c4ccc(-c5cccc6c5sc5ccccc56)cc4c4c5sc6ccccc6c5ccc43)n2)cc1 ; c1ccc(-c2ccc(-c3ccc(-c4ccnc(-n5c6ccccc6c6c7sc8ccccc8c7ccc65)n4)cc3)cc2)cc1 ; c1ccc(-c2nc(-n3c4cc(-c5cccc6c5oc5ccccc56)ccc4c4c5oc6ccccc6c5ccc43)nc3ccccc23)cc1]

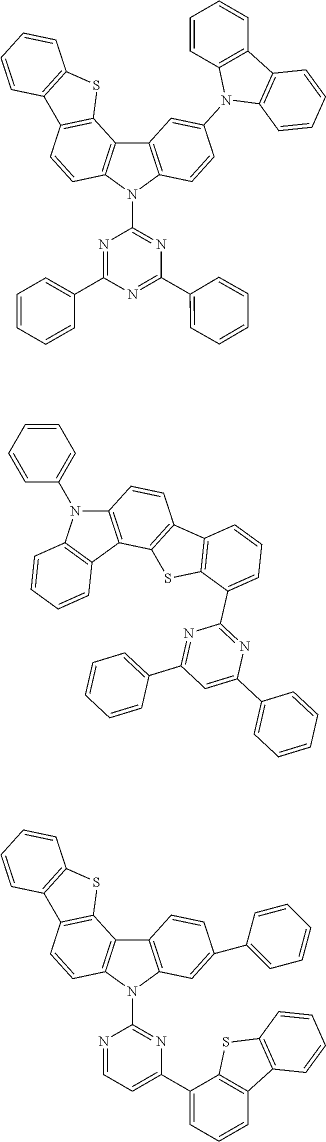 [SMILES: c1ccc(-c2nc(-c3ccccc3)nc(-n3c4ccc(-n5c6ccccc6c6ccccc65)cc4c4c5sc6ccccc6c5ccc43)n2)cc1 ; c1ccc(-c2cc(-c3ccccc3)nc(-c3cccc4c3sc3c4ccc4c3c3ccccc3n4-c3ccccc3)n2)cc1 ; c1ccc(-c2ccc3c4c5sc6ccccc6c5ccc4n(-c4nccc(-c5cccc6c5sc5ccccc56)n4)c3c2)cc1]

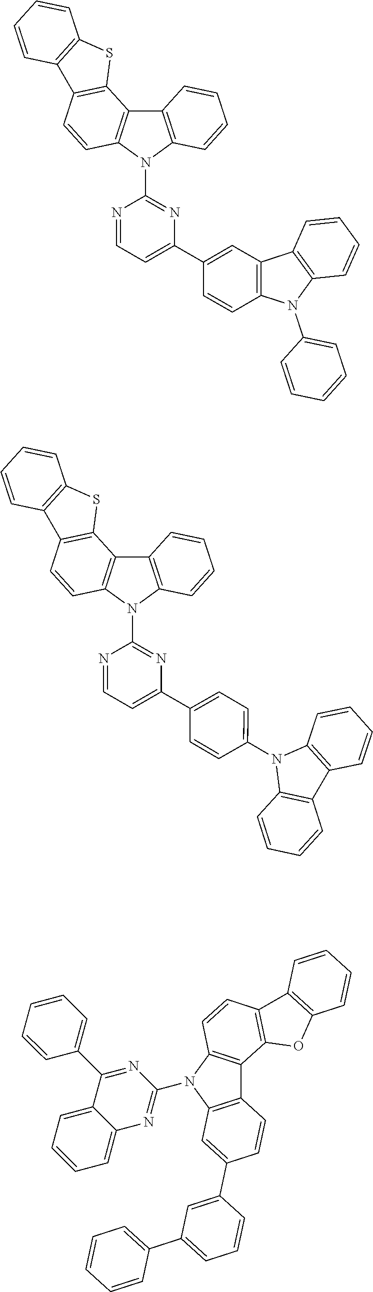 [SMILES: c1ccc(-n2c3ccccc3c3cc(-c4ccnc(-n5c6ccccc6c6c7sc8ccccc8c7ccc65)n4)ccc32)cc1 ; c1ccc2c(c1)sc1c2ccc2c1c1ccccc1n2-c1nccc(-c2ccc(-n3c4ccccc4c4ccccc43)cc2)n1 ; c1ccc(-c2cccc(-c3ccc4c5c6oc7ccccc7c6ccc5n(-c5nc(-c6ccccc6)c6ccccc6n5)c4c3)c2)cc1]

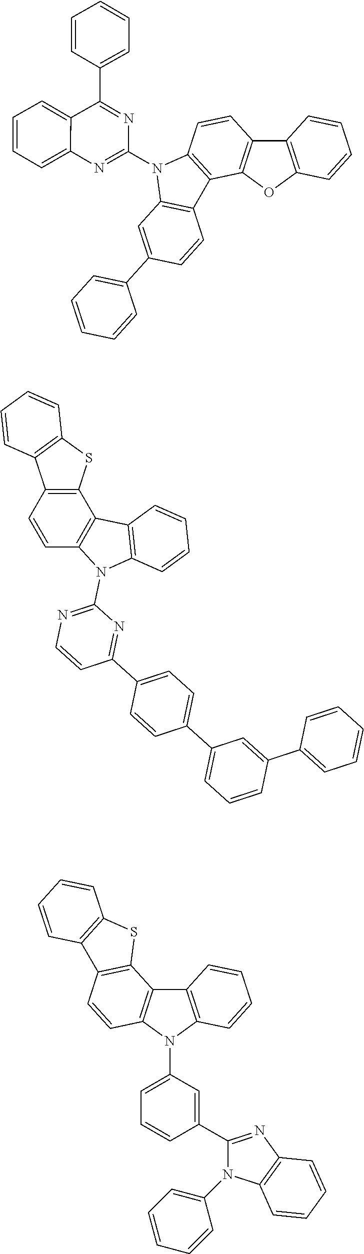 [SMILES: c1ccc(-c2ccc3c4c5oc6ccccc6c5ccc4n(-c4nc(-c5ccccc5)c5ccccc5n4)c3c2)cc1 ; c1ccc(-c2cccc(-c3ccc(-c4ccnc(-n5c6ccccc6c6c7sc8ccccc8c7ccc65)n4)cc3)c2)cc1 ; c1ccc(-n2c(-c3cccc(-n4c5ccccc5c5c6sc7ccccc7c6ccc54)c3)nc3ccccc32)cc1]

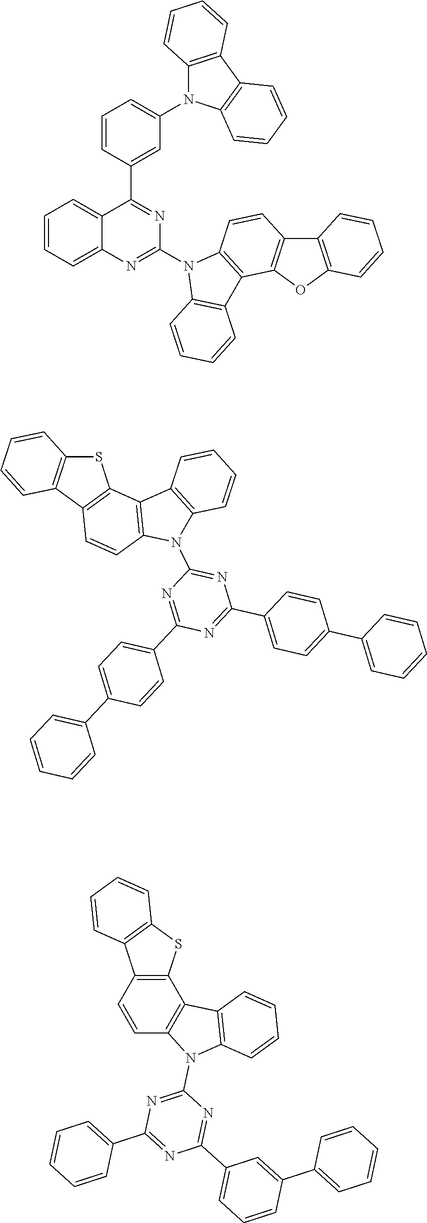 [SMILES: c1cc(-c2nc(-n3c4ccccc4c4c5oc6ccccc6c5ccc43)nc3ccccc23)cc(-n2c3ccccc3c3ccccc32)c1 ; c1ccc(-c2ccc(-c3nc(-c4ccc(-c5ccccc5)cc4)nc(-n4c5ccccc5c5c6sc7ccccc7c6ccc54)n3)cc2)cc1 ; c1ccc(-c2cccc(-c3nc(-c4ccccc4)nc(-n4c5ccccc5c5c6sc7ccccc7c6ccc54)n3)c2)cc1]

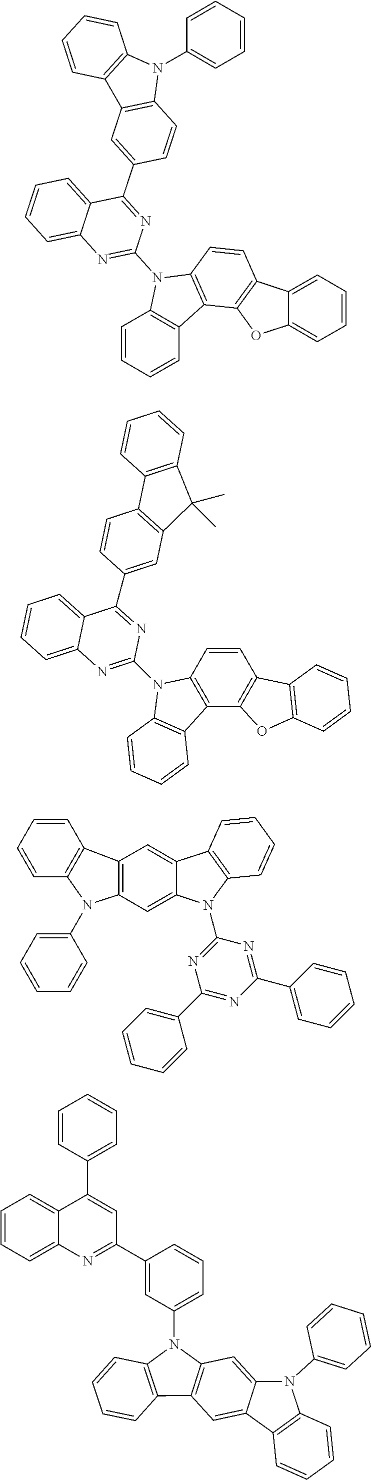

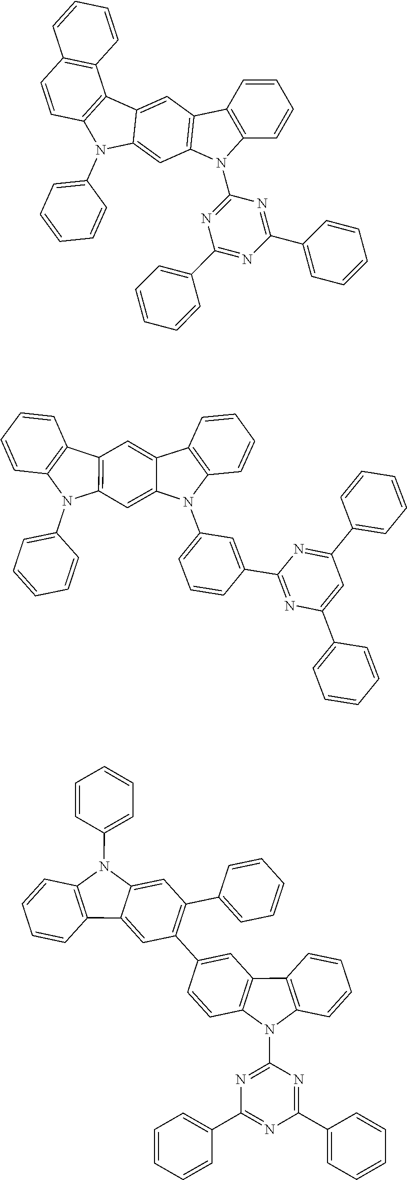 [SMILES: c1ccc(-c2nc(-c3ccccc3)nc(-n3c4ccccc4c4cc5c6c7ccccc7ccc6n(-c6ccccc6)c5cc43)n2)cc1 ; c1ccc(-c2cc(-c3ccccc3)nc(-c3cccc(-n4c5ccccc5c5cc6c7ccccc7n(-c7ccccc7)c6cc54)c3)n2)cc1 ; c1ccc(-c2nc(-c3ccccc3)nc(-n3c4ccccc4c4cc(-c5cc6c7ccccc7n(-c7ccccc7)c6cc5-c5ccccc5)ccc43)n2)cc1]

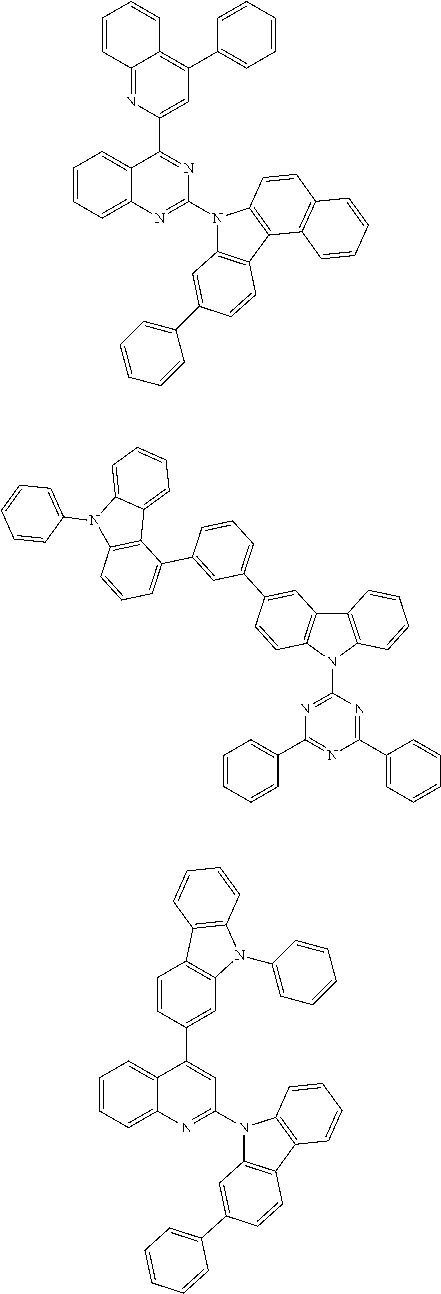 [SMILES: c1ccc(-c2ccc3c4c5ccccc5ccc4n(-c4nc(-c5cc(-c6ccccc6)c6ccccc6n5)c5ccccc5n4)c3c2)cc1 ; c1ccc(-c2nc(-c3ccccc3)nc(-n3c4ccccc4c4cc(-c5cccc(-c6cccc7c6c6ccccc6n7-c6ccccc6)c5)ccc43)n2)cc1 ; c1ccc(-c2ccc3c4ccccc4n(-c4cc(-c5ccc6c7ccccc7n(-c7ccccc7)c6c5)c5ccccc5n4)c3c2)cc1]

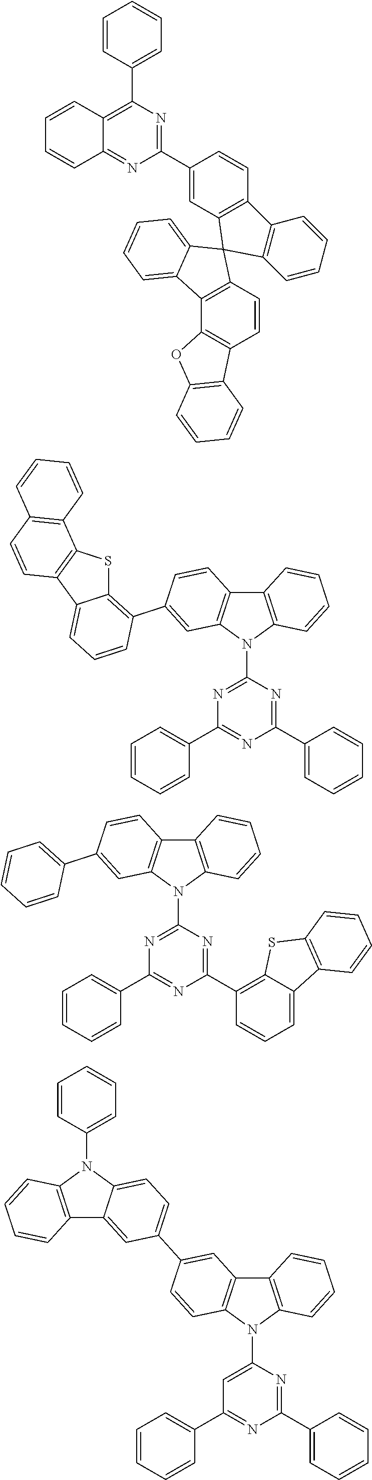

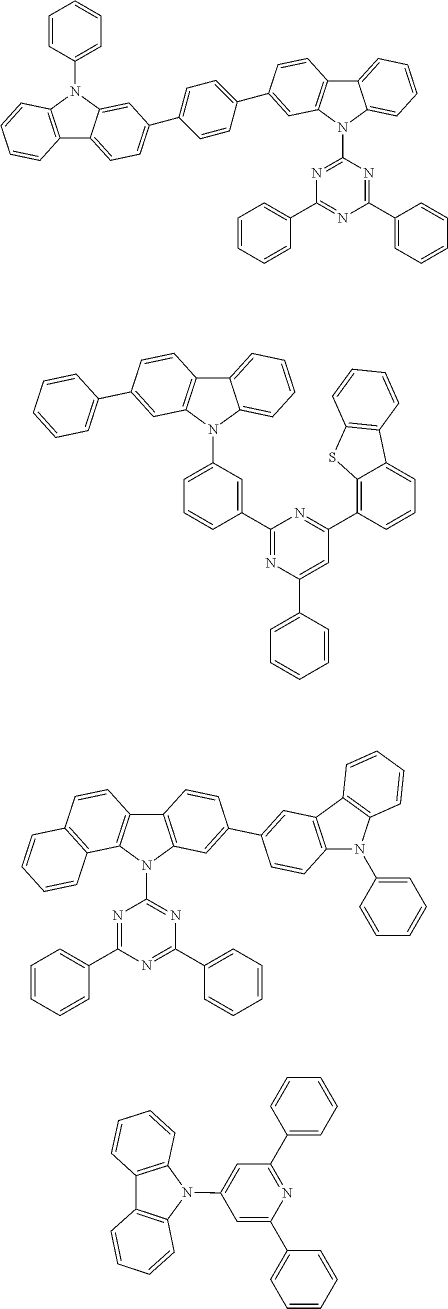 [SMILES: c1ccc(-c2nc(-c3ccccc3)nc(-n3c4ccccc4c4ccc(-c5ccc(-c6ccc7c8ccccc8n(-c8ccccc8)c7c6)cc5)cc43)n2)cc1 ; c1ccc(-c2ccc3c4ccccc4n(-c4cccc(-c5nc(-c6ccccc6)cc(-c6cccc7c6sc6ccccc67)n5)c4)c3c2)cc1 ; c1ccc(-c2nc(-c3ccccc3)nc(-n3c4cc(-c5ccc6c(c5)c5ccccc5n6-c5ccccc5)ccc4c4ccc5ccccc5c43)n2)cc1 ; c1ccc(-c2cc(-n3c4ccccc4c4ccccc43)cc(-c3ccccc3)n2)cc1]

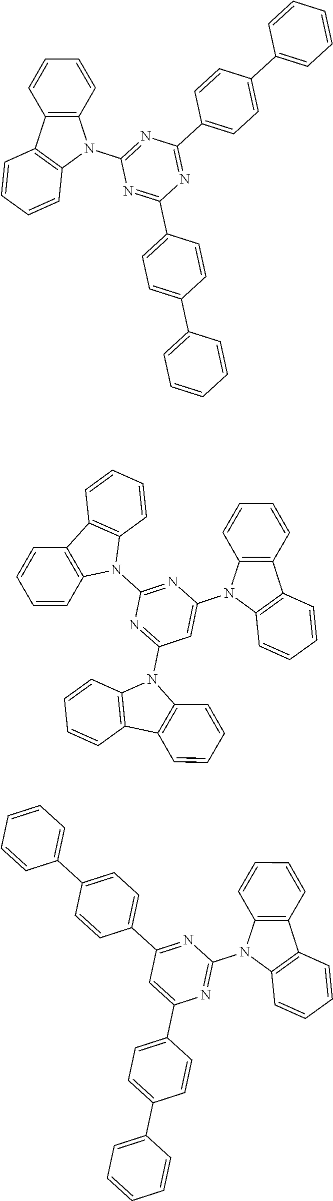 [SMILES: c1ccc(-c2ccc(-c3nc(-c4ccc(-c5ccccc5)cc4)nc(-n4c5ccccc5c5ccccc54)n3)cc2)cc1 ; c1ccc2c(c1)c1ccccc1n2-c1cc(-n2c3ccccc3c3ccccc32)nc(-n2c3ccccc3c3ccccc32)n1 ; c1ccc(-c2ccc(-c3cc(-c4ccc(-c5ccccc5)cc4)nc(-n4c5ccccc5c5ccccc54)n3)cc2)cc1]

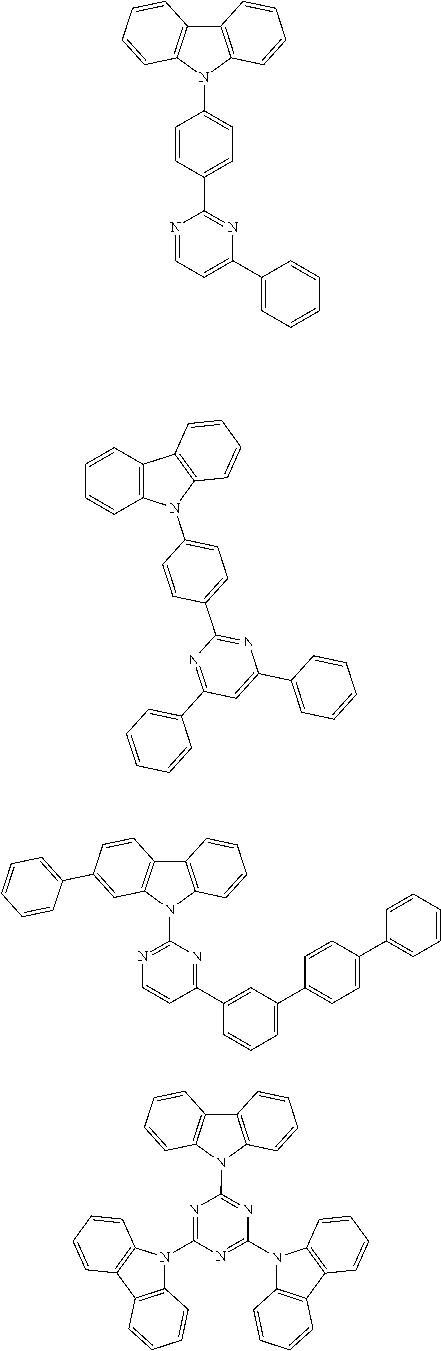

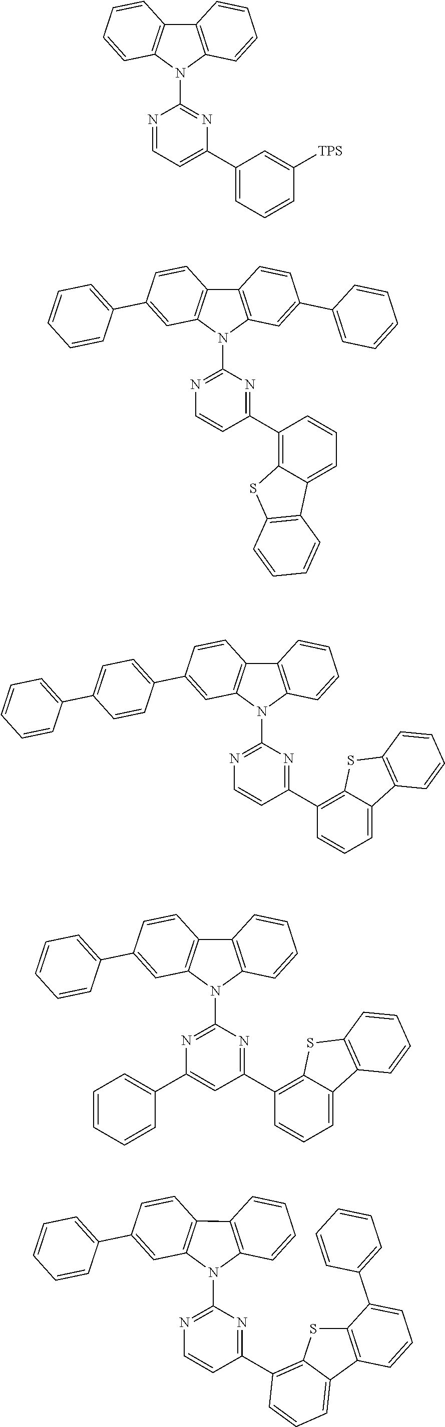

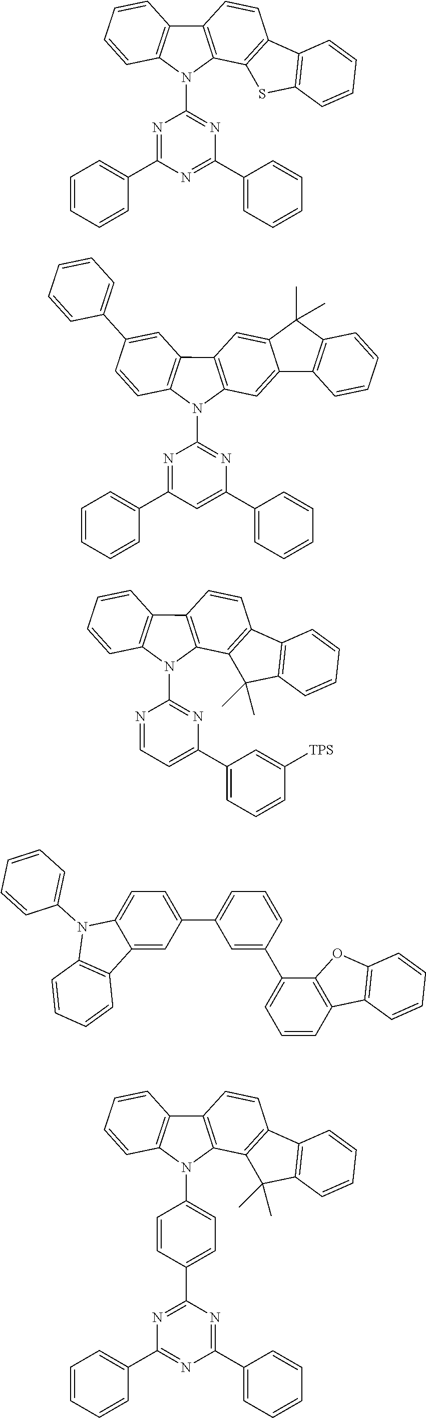

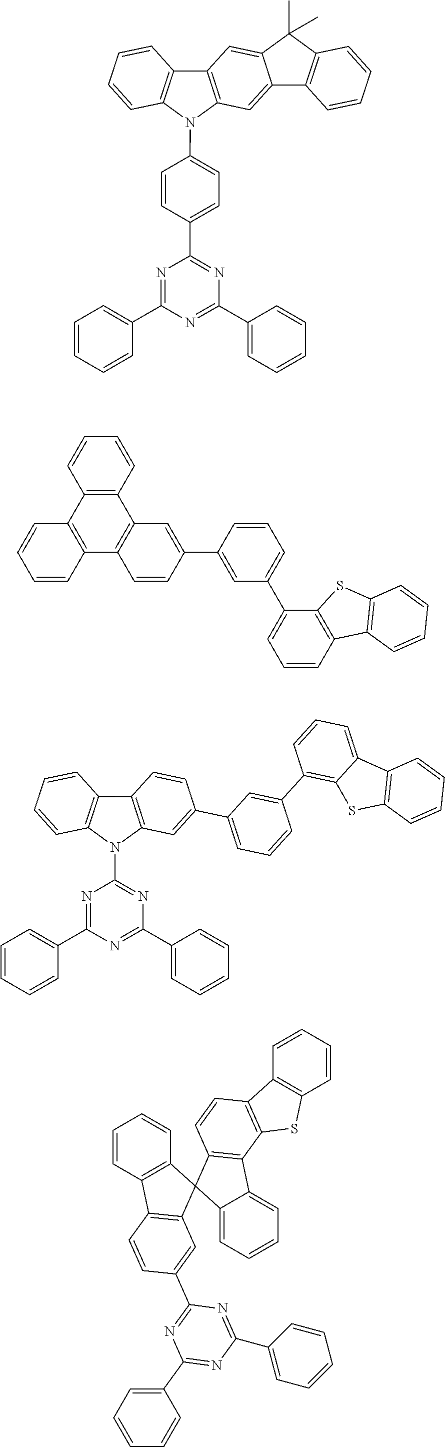 [SMILES: CC1(C)c2ccccc2-c2cc3c(cc21)c1ccccc1n3-c1ccc(-c2nc(-c3ccccc3)nc(-c3ccccc3)n2)cc1 ; c1cc(-c2ccc3c4ccccc4c4ccccc4c3c2)cc(-c2cccc3c2sc2ccccc23)c1 ; c1ccc(-c2nc(-c3ccccc3)nc(-n3c4ccccc4c4ccc(-c5cccc(-c6cccc7c6sc6ccccc67)c5)cc43)n2)cc1 ; c1ccc(-c2nc(-c3ccccc3)nc(-c3ccc4c(c3)C3(c5ccccc5-4)c4ccccc4-c4c3ccc3c4sc4ccccc43)n2)cc1]

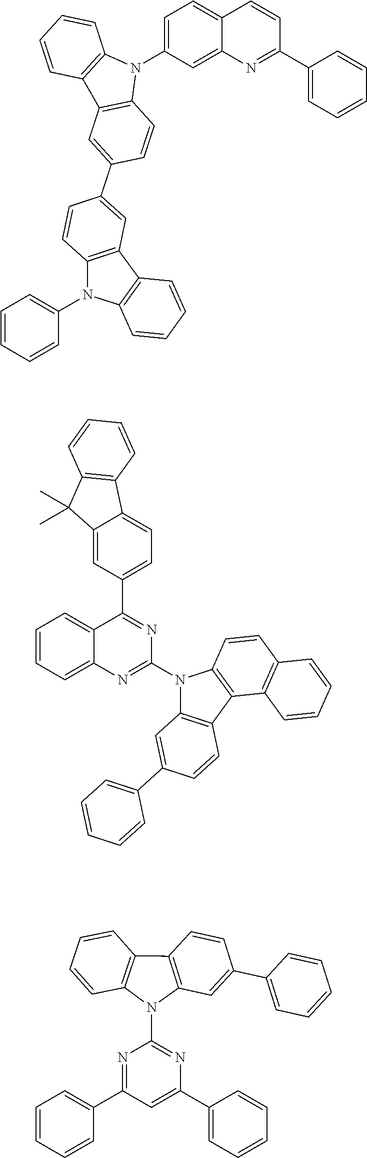 [SMILES: c1ccc(-c2ccc3ccc(-n4c5ccccc5c5cc(-c6ccc7c(c6)c6ccccc6n7-c6ccccc6)ccc54)cc3n2)cc1 ; CC1(C)c2ccccc2-c2ccc(-c3nc(-n4c5cc(-c6ccccc6)ccc5c5c6ccccc6ccc54)nc4ccccc34)cc21 ; c1ccc(-c2ccc3c4ccccc4n(-c4nc(-c5ccccc5)cc(-c5ccccc5)n4)c3c2)cc1]

In another embodiment of the present invention, a material used for an organic electroluminescent device is provided. The material comprises the organic electroluminescent compound according to the present invention as a host material or a hole transport material.

In addition, the organic electroluminescent device according to the present invention comprises a first electrode; a second electrode; and at least one organic layer between said first and second electrodes. Said organic layer comprises a light emitting layer. Said light emitting layer comprises the organic electroluminescent composition according to the present invention and the phosphorescent dopant material. Said organic electroluminescent composition is used as a host material.

The organic electroluminescent device according to the present invention may further comprise, in addition to the organic electroluminescent compound represented by formula 1, at least one compound selected from the group consisting of arylamine-based compounds and styrylarylamine-based compounds.

In the organic electroluminescent device according to the present invention, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal. The organic layer may further comprise at least one additional light-emitting layer and a charge generating layer.

In addition, the organic electroluminescent device according to the present invention may emit white light by further comprising at least one light-emitting layer which comprises a blue electroluminescent compound, a red electroluminescent compound or a green electroluminescent compound known in the field, besides the compound according to the present invention. Also, if needed, a yellow or orange light-emitting layer can be comprised in the device.

According to the present invention, at least one layer (hereinafter, “a surface layer”) may be preferably placed on an inner surface(s) of one or both electrode(s); selected from a chalcogenide layer, a metal halide layer and a metal oxide layer. Specifically, a chalcogenide(includes oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, said chalcogenide includes SiO_{X} (1≦X≦2), AlO_{X} (1≦X≦1.5), SiON, SiAlON, etc.; said metal halide includes LiF, MgF₂, CaF₂, a rare earth metal fluoride, etc.; and said metal oxide includes Cs₂O, Li₂O, MgO, SrO, BaO, CaO, etc.

Preferably, in the organic electroluminescent device according to the present invention, a mixed region of an electron transport compound and an reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge generating layer to prepare an electroluminescent device having two or more electroluminescent layers and emitting white light.

In order to form each layer of the organic electroluminescent device according to the present invention, dry film-forming methods such as vacuum evaporation, sputtering, plasma and ion plating methods, or wet film-forming methods such as spin coating, dip coating, flow coating methods can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

Hereinafter, the compound, the preparation method of the compound, and the luminescent properties of the device will be explained in detail with reference to the following examples.

### EXAMPLE 1

### Preparation of Compound C-9

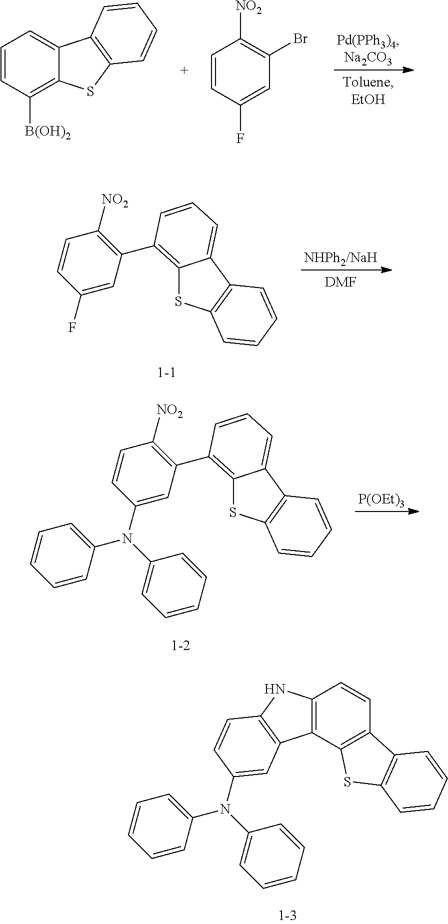

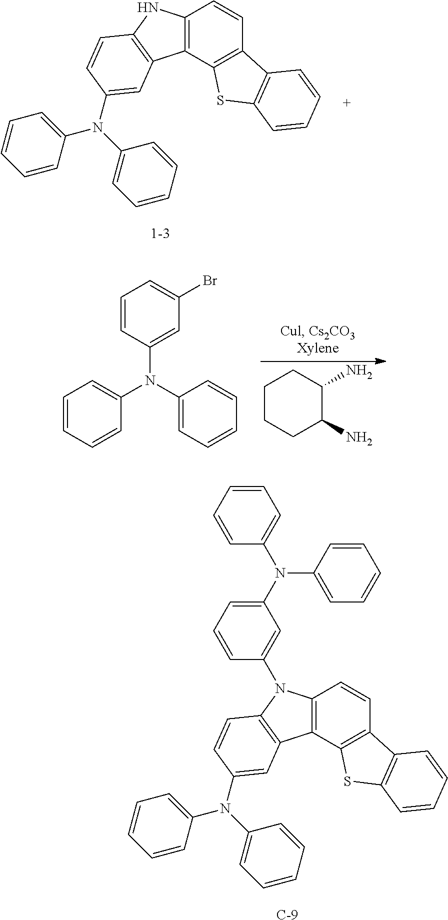

Preparation of Compound 1-3

After adding 4-bromo-2-fluoronitrobenzene 50 g (230 mmol), 4-dibenzothiophene-boronic acid 63 g (276 mmol), Pd(PPh₃)₄ 5 g (4.6 mmol), and Na₂CO₃ 61 g (575 mmol) to a mixture solvent of toluene 600 mL, and EtOH 200 mL, and adding H₂O 200 mL to the mixture, the mixture was stirred at 120° C. for 2 hours. After completing the reaction, the mixture was washed with distilled water, and extracted with ethyl acetate (EA). Then, the organic layer was dried with MgSO₄, and the solvent was removed with a rotary evaporator. Then, the remaining product was purified with a column to obtain compound 1-1, 65 g (87%).

After dissolving diphenylamine 34 g (200 mmol) in dimethylformamide (DMF) 800 mL, NaH 13.2 g (60% in mineral oil, 220 mmol) was slowly added to the mixture. After stirring the mixture for 15 minutes, compound 1-1, 24 g dissolved in DMF 200 mL was slowly added dropwise to the solution. The reaction solution was stirred at room temperature for 4 hours, and quenched with methanol. Then, the obtained product was worked up with EA/H₂O, and purified with a column to obtain compound 1-2, 85 g (90%).

After mixing compound 1-2 85 g (180 mmol), P(OEt)₃ 450 mL, and 1,2-dichlorobenzene 450 mL, the mixture was stirred at 150° C. for 8 hours. After the reaction, solvent was removed, and the remaining product was purified with a column to obtain compound 1-3, 38 g (48%).

Preparation of Compound C-9

After adding compound 1-3 7 g (16 mmol), triphenylamine-3-bromide 5.7 g (17.5 mmol), CuI 1.5 g (8 mmol), trans-diaminocyclohexane 3.5 mL (32 mmol), and cesium carbonate 10 g (32 mmol) to xylene 100 mL, the mixture was refluxed for 3 hours. After cooling the reaction mixture to room temperature, the obtained solid was filtered and washed with methylenechloride (MC). The remaining solution was distilled under reduced pressure, and purified with a column to obtain compound C-9, 7.9 g (72%).

MS/FAB found 683.8; calculated 683.24

### EXAMPLE 2

### Preparation of Compound C-8

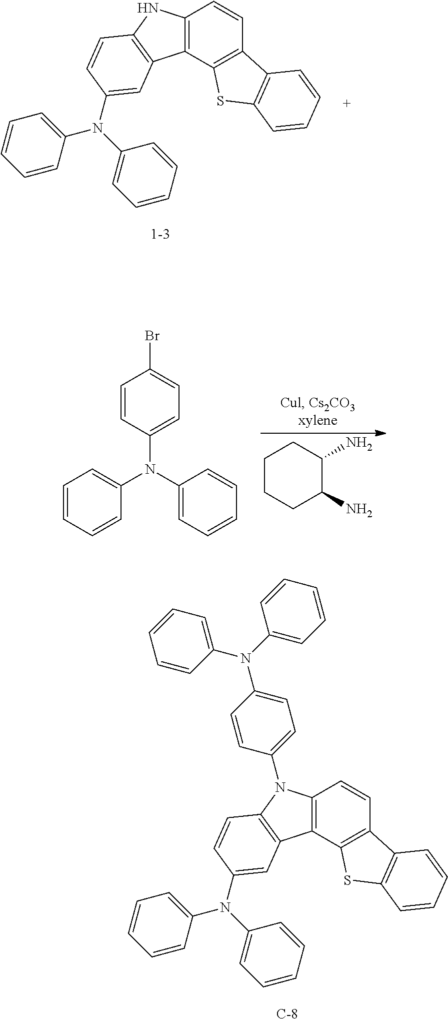

After adding compound 1-3 8.6 g (19.6 mmol), triphenylamine-4-bromide 7 g (21.6 mmol), CuI 1.9 g (10 mmol), trans-diaminocyclohexane 4.5 mL (39 mmol), and cesium carbonate 12.8 g (39 mmol) to xylene 100 mL, the mixture was refluxed for 3 hours. After cooling the reaction mixture to room temperature, the obtained solid was filtered and washed with MC. The remaining solution was distilled under reduced pressure, and purified with a column to obtain compound C-8, 8.9 g (66%).

MS/FAB found 683.8; calculated 683.24

### EXAMPLE 3

### Preparation of Compound C-118

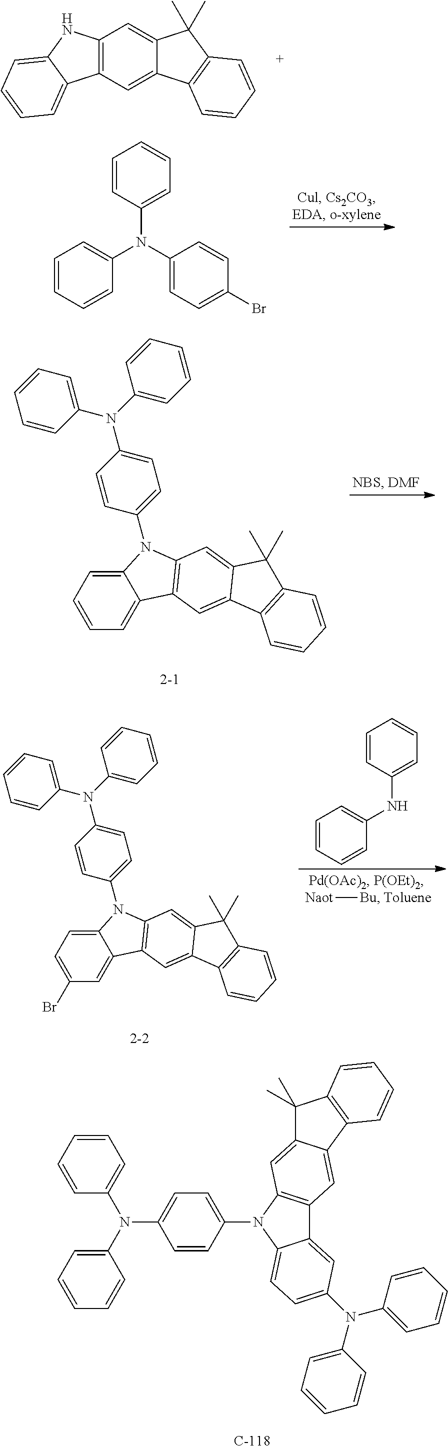

After adding o-xylene 200 mL to a mixture of 7,7-dimethyl-5,7-dihydroindeno[2,1-b]carbazole 7 g (0.024 mol), 4-bromo-N,N-diphenylaniline 8.8 g (0.027 mol), CuI 2.3 g (0.012 mol), ethylenediamine 1.6 mL (0.024 mol), and Cs₂CO₃ 24 g (0.081 mol), the mixture was stirred at 150° C. for 4 hours. After completing the reaction, the mixture was washed with distilled water, and extracted with EA. Then, the organic layer was dried with MgSO₄, and solvent was removed. Then, the remaining product was purified with a column to obtain compound 2-1, 6.4 g (50%).

After adding compound 2-1, 6.4 g (0.012 mol) to DMF 1.4 L, the mixture was stirred at 0° C. for 10 minutes. Then, N-bromosuccinimide (NBS) 2.0 g (0.011 mol) was added to DMF 100 mL to be dissolved, and slowly added to the mixture. The mixture was stirred at 0° C. for 6 hours. After completing the reaction, the mixture was neutralized with distilled water, and extracted with EA. Then, the organic layer was dried with MgSO₄, and the solvent was removed. Then, the remaining product was purified with a column to obtain compound 2-2, 7.2 g (98%).

After adding toluene 150 mL to a mixture of compound 2-2 7.2 g (0.011 mol), diphenylamine 2.2 g (0.013 mol), Pd(OAc)₂ 800 mg (0.3 mmol), P(t-Bu)₃ 0.5 mL (0.001 mol), and NaOt-Bu 3.4 g (0.033 mol), the mixture was stirred at 120° C. for 3 hours. After the reaction, the mixture was washed with distilled water, and extracted with EA. Then, the organic layer was dried with MgSO₄, and the solvent was removed. Then, the remaining product was purified with a column to obtain compound C-118, 5.7 g (69%).

MS/FAB found 693.9; calculated 693.31

### DEVICE EXAMPLE 1

### Production of an OLED Device Using the Organic Electroluminescent Compound According to the Present Invention

An OLED device was produced using the light emitting material according to the present invention. A transparent electrode indium tin oxide (ITO) thin film (15 Ω/sq) on a glass substrate for an organic light-emitting diode (OLED) device (Samsung Corning, Republic of Korea) was subjected to an ultrasonic washing with trichloroethylene, acetone, ethanol and distilled water, sequentially, and then was stored in isopropanol. Then, the ITO substrate was mounted on a substrate holder of a vacuum vapor depositing apparatus. N¹,N^{1′}-([1,1′-biphenyl]-4,4′-diyl)bis(N¹-(naphthalen-1-yl)-N⁴,N⁴-diphenylbenzen-1,4-diamine) was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a hole injection layer having a thickness of 60 nm on the ITO substrate. Then, organic electroluminescent compound C-118 according to the present invention was introduced into another cell of said vacuum vapor depositing apparatus, and was evaporated by applying an electric current to the cell, thereby forming a hole transport layer having a thickness of 20 nm on the hole injection layer. Thereafter, 9-(3-(4,6-biphenyl-1,3,5-triazin-2-yl)phenyl)-9′-phenyl-9H,9′H-3,3′-bicarbazole was introduced into one cell of the vacuum vapor depositing apparatus, as a host material, and compound D-1 was introduced into another cell as a dopant. The two materials were evaporated at different rates and were deposited in a doping amount of 15 wt % based on the total amount of the host and dopant to form a light-emitting layer having a thickness of 30 nm on the hole transport layer. Then, 2-(4-(9,10-di(naphthalen-2-yl)anthracen-2-yl)phenyl)-1-phenyl-1H-benzo[d]imidazole was introduced into one cell and lithium quinolate was introduced into another cell. The two materials were evaporated at the same rate and were deposited in a doping amount of 50 wt % each to form an electron transport layer having a thickness of 30 nm on the light-emitting layer. Then, after depositing lithium quinolate as an electron injection layer having a thickness of 2 nm on the electron transport layer, an Al cathode having a thickness of 150 nm was deposited by another vacuum vapor deposition apparatus on the electron injection layer. Thus, an OLED device was produced. All the materials used for producing the OLED device were purified by vacuum sublimation at 10⁻⁶ torr prior to use.

The produced OLED device showed a green emission having a luminance of 11084 cd/m² and a current density of 26.1 mA/cm².

### DEVICE EXAMPLE 2

### Production of an OLED Device Using the Organic Electroluminescent Compound According to the Present Invention

An OLED device was produced in the same manner as in Device Example 1, except for depositing the hole transport layer using compound C-8 having a thickness of 20 nm; introducing 9-(4-([1,1′-biphenyl]-3-yl)quinazolin-2-yl)-9′-phenyl-9H,9′H-3,3′-bicarbazole into a cell of a vacuum vapor depositing apparatus; introducing compound D-50 as a dopant into another cell; and evaporating the two materials at different rates in a doping amount of 3 wt % based on the total amount of the host and dopant to form a light-emitting layer having a thickness of 30 nm on the hole transport layer.

The produced OLED device showed a red emission having a luminance of 1188 cd/m² and a current density of 8.0 mA/cm².

### DEVICE EXAMPLE 3

### Production of an OLED Device Using the Organic Electroluminescent Compound According to the Present Invention

An OLED device was produced in the same manner as in Device Example 1, except for depositing the hole transport layer using compound C-9 having a thickness of 20 nm; introducing 3-([1,1′-biphenyl]-4-yl)-5-(4-phenylquinazolin-2-yl)-5H-benzofuro[3,2-c]carbazole into a cell of a vacuum vapor depositing apparatus; introducing compound D-37 as a dopant into another cell; and evaporating the two materials at different rates in a doping amount of 3 wt % based on the total amount of the host and dopant to form a light-emitting layer having a thickness of 30 nm on the hole transport layer.

The produced OLED device showed a red emission having a luminance of 2610 cd/m² and a current density of 16.5 mA/cm².

### COMPARATIVE EXAMPLE 1

### Production of an OLED Device Using Conventional Light Emitting Material

An OLED device was produced in the same manner as in Device Example 1, except for evaporating N,N′-di(4-biphenyl)-N,N′-di(4-biphenyl)-4,4′-diaminobiphenyl as a hole transport material to form a hole transport layer having a thickness of 20 nm; using 4,4′-N,N′-dicarbazole-biphenyl as a host material, compound D-15 as a dopant to form a light-emitting layer having a thickness of 30 nm on the hole transport layer; and depositing aluminum(III)bis(2-methyl-8-quinolinato)4-phenylphenolate to form a hole blocking layer having a thickness of 10 nm.

The produced OLED device showed a green emission having a luminance of 1550 cd/m² and a current density of 4.50 mA/cm².

### COMPARATIVE EXAMPLE 2

### Production of an OLED Device Using Conventional Light Emitting Material

An OLED device was produced in the same manner as in Comparative Example 1, except for using compound D-50 as a dopant.

The produced OLED device showed a red emission having a luminance of 2240 cd/m² and a current density of 48.7 mA/cm².

It is verified that the organic electroluminescent compounds of the present invention have superior luminous characteristics over conventional materials. In addition, the devices using the organic electroluminescent compounds according to the present invention have superior luminous characteristics.

1. An organic electroluminescent compound represented by the following formula 1: 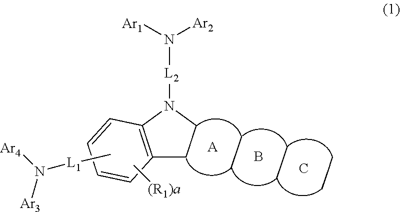
wherein
ring A represents an aromatic ring of 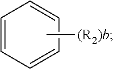
ring B represents a pentacyclic ring of 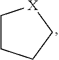
where X represents —O—, —S—, —N(R₄)—, —C(R₅)(R₆)— or —Si(R₇)(R₈)—;
ring C represents an aromatic ring of 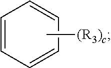
L₁ represents a single bond, a substituted or unsubstituted 5- to 30-membered heteroarylene, or a substituted or unsubstituted (C6-C30)arylene;
L₂ represents a substituted or unsubstituted 5- to 30-membered heteroarylene, or a substituted or unsubstituted (C6-C30)arylene;
Ar₁ to Ar₄ each independently represent a substituted or unsubstituted 5- to 30-membered heteroaryl, or a substituted or unsubstituted (C6-C30)aryl;
R₁ to R₃ each independently represent hydrogen, deuterium, a halogen, a substituted or unsubstituted (C1-C30)alkyl, a substituted or unsubstituted (C6-C30)aryl, a substituted or unsubstituted 5- to 30-membered heteroaryl, —NR₁₁R₁₂ or —SiR₁₃R₁₄R₁₅; or are linked to an adjacent substituent(s) to form a mono- or polycyclic, 3- to 30-membered alicyclic or aromatic ring whose carbon atom(s) may be replaced with at least one hetero atom selected from the group consisting of nitrogen, oxygen and sulfur;
R₄ to R₈, and R₁₁ to R₁₅ each independently represent hydrogen, deuterium, a halogen, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic, 3- to 30-membered alicyclic or aromatic ring;
a represents an integer of 1 to 3; where a is an integer of 2 or more, each of the substituent may be same or different;
b represents 1 or 2; where b is 2, each of the substituent may be same or different;
c represents an integer of 1 to 4; where c is an integer of 2 or more, each of the substituent may be same or different; and
the heteroaryl(ene) contains at least one hetero atom selected from B, N, O, S, P(═O), Si and P.

2. The compound according to claim 1, wherein the compound represented by formula 1 is represented by one selected from formulae 2 to 4: 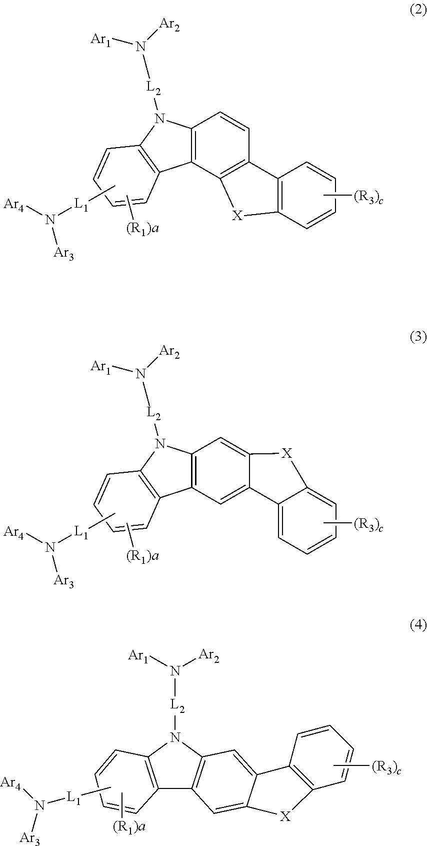
wherein X, L₁, L₂, Ar₁ to Ar₄, R₁, R₃, a and c are as defined in claim 1.

3. The compound according to claim 1, wherein the substituents of the substituted alkyl, the substituted aryl(ene), and the substituted heteroaryl(ene) in L₁, L₂, Ar₁ to Ar₄, R₁ to R₈, and R₁₁ to R₁₅ each independently are at least one selected from the group consisting of deuterium, a halogen, a cyano, a carboxyl, a nitro, a hydroxyl, a (C1-C30)alkyl, a halo(C1-C30)alkyl, a (C6-C30)aryl, a 5- to 30-membered heteroaryl, a 5- to 30-membered heteroaryl substituted with a (C6-C30)aryl, a (C6-C30)aryl substituted with a 5- to 30-membered heteroaryl, a (C3-C30)cycloalkyl, a 3- to 7-membered heterocycloalkyl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, a (C2-C30)alkenyl, a (C2-C30)alkynyl, a mono- or di-(C1-C30)alkylamino, a mono- or di-(C6-C30)arylamino, a (C1-C30)alkyl(C6-C30)arylamino, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

4. The compound according to claim 1, wherein X represents —O—, —S—, —N(R₄)— or —C(R₅)(R₆)—, where R₄ represents a substituted or unsubstituted (C6-C15)aryl, and R₅ and R₆ each independently represent a substituted or unsubstituted (C1-C10)alkyl, or a substituted or unsubstituted (C6-C15)aryl;
L₁ represents a single bond, or a substituted or unsubstituted (C6-C15)arylene;
L₂ represents a substituted or unsubstituted (C6-C15)arylene;
Ar₁ to Ar₄ each independently represent a substituted or unsubstituted 5- to 15-membered heteroaryl, or a substituted or unsubstituted (C6-C20)aryl; and
R₁ to R₃ each independently represent hydrogen, a substituted or unsubstituted (C1-C10)alkyl, a substituted or unsubstituted (C6-C15)aryl, a substituted or unsubstituted 5- to 15-membered heteroaryl, —NR₁₁R₁₂ or —SiR₁₃R₁₄R₁₅, where R₁₁ and R₁₂ each independently represent a substituted or unsubstituted (C6-C15)aryl, and R₁₃ to R₁₅ each independently represent a substituted or unsubstituted (C1-C10)alkyl.

5. The compound according to claim 1, wherein X represents —O—, —S—, —N(R₄)— or —C(R₅)(R₆)—, where R₄ represents an unsubstituted (C6-C15)aryl, and R₅ and R₆ each independently represent an unsubstituted (C1-C10)alkyl, or an unsubstituted (C6-C15)aryl;
L₁ represents a single bond, an unsubstituted (C6-C15)arylene, or a (C6-C15)arylene substituted with a (C1-C6)alkyl or a (C6-C12)aryl;
L₂ represents an unsubstituted (C6-C15)arylene, or a (C6-C15)arylene substituted with a (C1-C6)alkyl or a (C6-C12)aryl;
Ar₁ to Ar₄ each independently represent an unsubstituted 5- to 15-membered heteroaryl, a 5- to 15-membered heteroaryl substituted with a (C6-C15)aryl, an unsubstituted (C6-C20)aryl, or a (C6-C20)aryl substituted with a (C1-C6)alkyl or a (C6-C15)aryl; and
R₁ to R₃ each independently represent hydrogen, an unsubstituted (C1-C10)alkyl, an unsubstituted (C6-C15)aryl, a (C6-C15)aryl substituted with a (C6-C15)aryl, an unsubstituted 5- to 15-membered heteroaryl, a 5- to 15-membered heteroaryl substituted with a (C1-C6)alkyl, —NR₁₁R₁₂ or —SiR₁₃R₁₄R₁₅, where R₁₁ and R₁₂ each independently represent an unsubstituted (C6-C15)aryl, and R₁₃ to R₁₅ each independently represent an unsubstituted (C1-C10)alkyl.

6. The compound according to claim 1, wherein the compound represented by formula 1 is selected from the group consisting of:

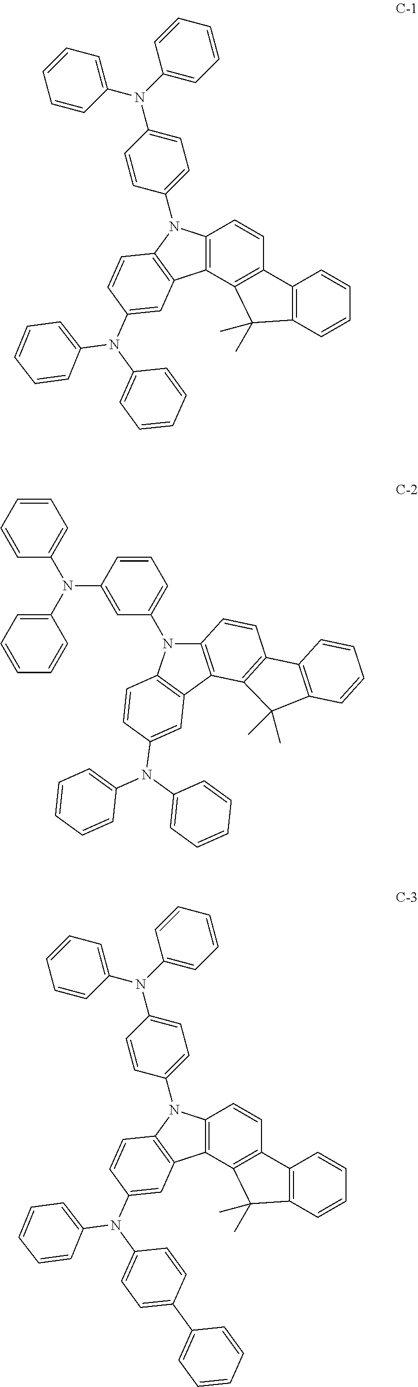 [SMILES: CC1(C)c2ccccc2-c2ccc3c(c21)c1cc(N(c2ccccc2)c2ccccc2)ccc1n3-c1ccc(N(c2ccccc2)c2ccccc2)cc1 ; CC1(C)c2ccccc2-c2ccc3c(c21)c1cc(N(c2ccccc2)c2ccccc2)ccc1n3-c1cccc(N(c2ccccc2)c2ccccc2)c1 ; CC1(C)c2ccccc2-c2ccc3c(c21)c1cc(N(c2ccccc2)c2ccc(-c4ccccc4)cc2)ccc1n3-c1ccc(N(c2ccccc2)c2ccccc2)cc1]

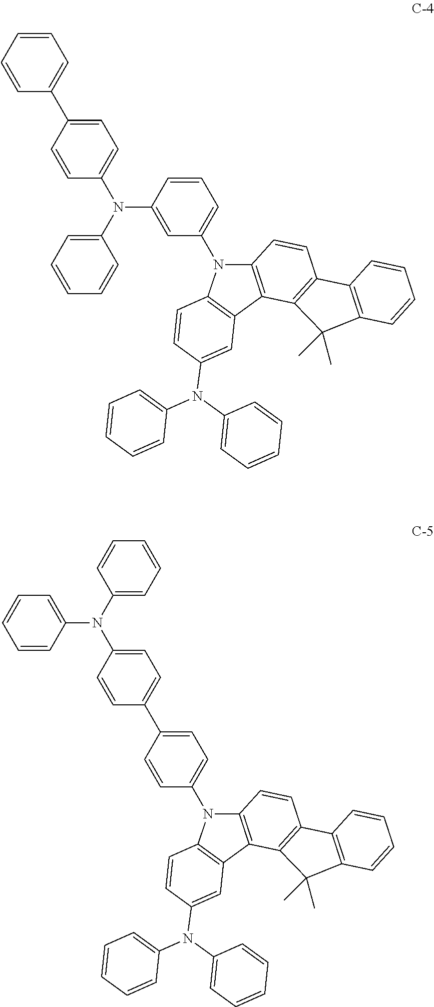 [SMILES: CC1(C)c2ccccc2-c2ccc3c(c21)c1cc(N(c2ccccc2)c2ccccc2)ccc1n3-c1cccc(N(c2ccccc2)c2ccc(-c3ccccc3)cc2)c1 ; CC1(C)c2ccccc2-c2ccc3c(c21)c1cc(N(c2ccccc2)c2ccccc2)ccc1n3-c1ccc(-c2ccc(N(c3ccccc3)c3ccccc3)cc2)cc1]

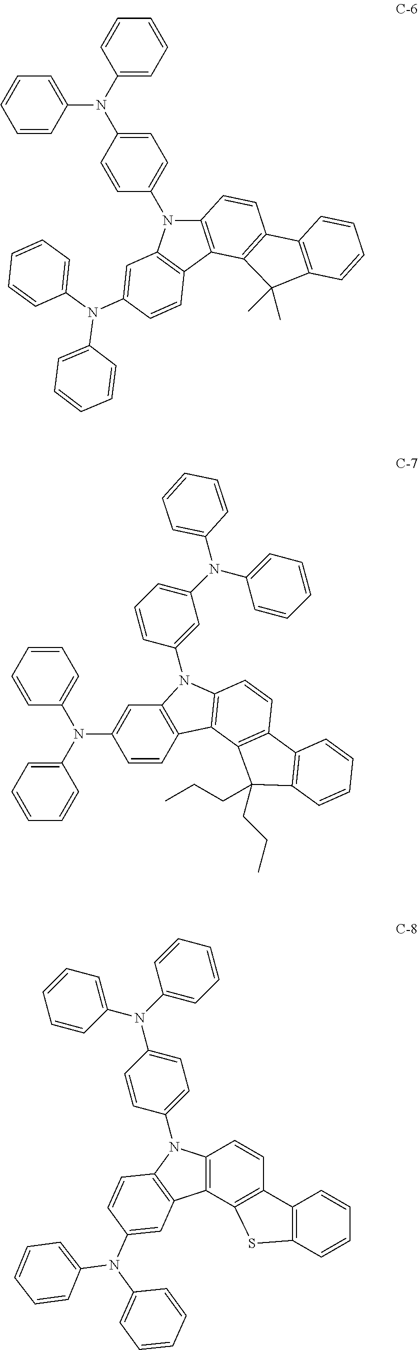 [SMILES: CC1(C)c2ccccc2-c2ccc3c(c21)c1ccc(N(c2ccccc2)c2ccccc2)cc1n3-c1ccc(N(c2ccccc2)c2ccccc2)cc1 ; CCCC1(CCC)c2ccccc2-c2ccc3c(c21)c1ccc(N(c2ccccc2)c2ccccc2)cc1n3-c1cccc(N(c2ccccc2)c2ccccc2)c1 ; c1ccc(N(c2ccccc2)c2ccc(-n3c4ccc(N(c5ccccc5)c5ccccc5)cc4c4c5sc6ccccc6c5ccc43)cc2)cc1]

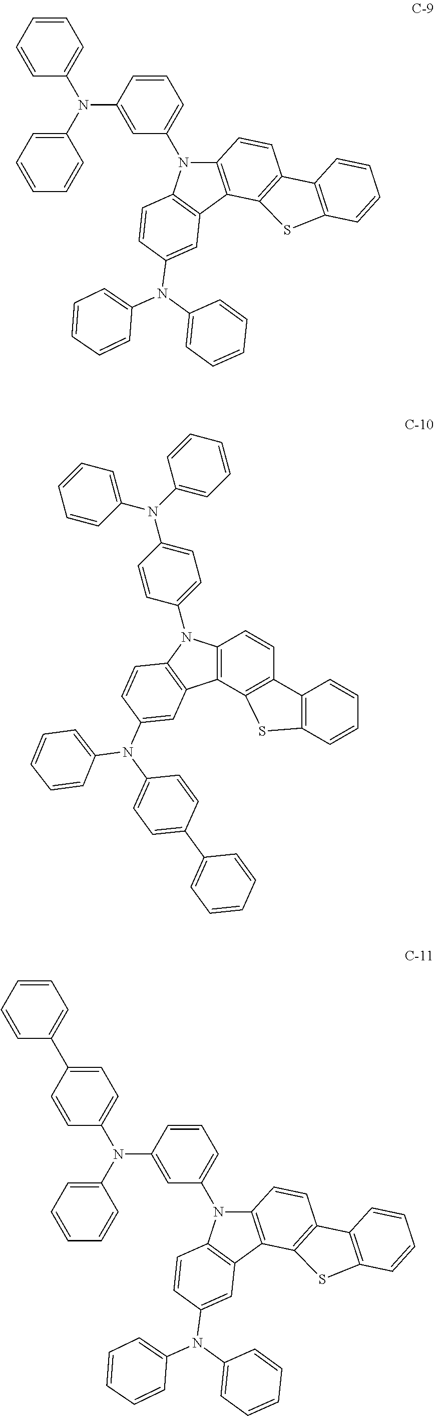 [SMILES: c1ccc(N(c2ccccc2)c2cccc(-n3c4ccc(N(c5ccccc5)c5ccccc5)cc4c4c5sc6ccccc6c5ccc43)c2)cc1 ; c1ccc(-c2ccc(N(c3ccccc3)c3ccc4c(c3)c3c5sc6ccccc6c5ccc3n4-c3ccc(N(c4ccccc4)c4ccccc4)cc3)cc2)cc1 ; c1ccc(-c2ccc(N(c3ccccc3)c3cccc(-n4c5ccc(N(c6ccccc6)c6ccccc6)cc5c5c6sc7ccccc7c6ccc54)c3)cc2)cc1]

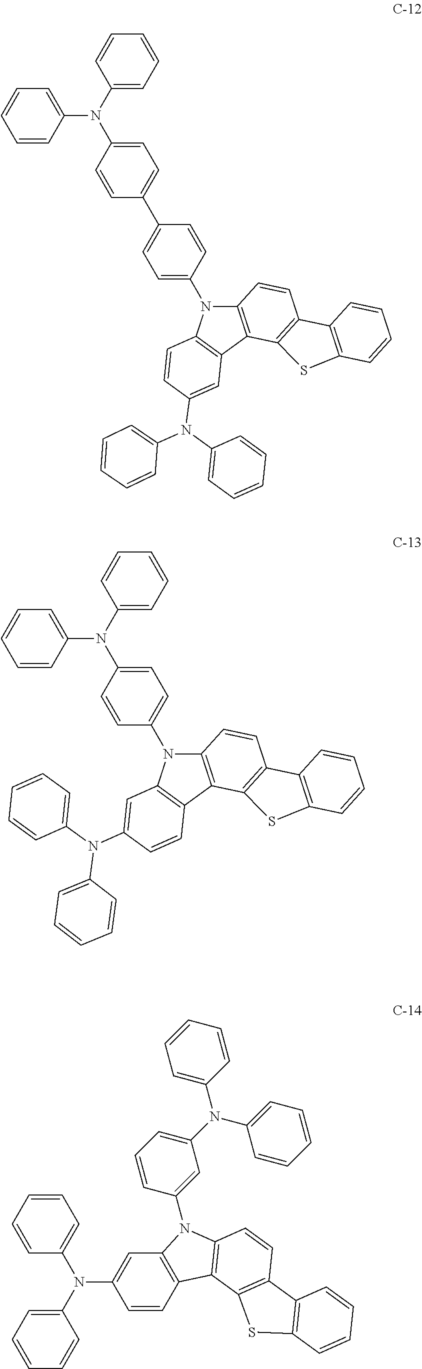 [SMILES: c1ccc(N(c2ccccc2)c2ccc(-c3ccc(-n4c5ccc(N(c6ccccc6)c6ccccc6)cc5c5c6sc7ccccc7c6ccc54)cc3)cc2)cc1 ; c1ccc(N(c2ccccc2)c2ccc(-n3c4cc(N(c5ccccc5)c5ccccc5)ccc4c4c5sc6ccccc6c5ccc43)cc2)cc1 ; c1ccc(N(c2ccccc2)c2cccc(-n3c4cc(N(c5ccccc5)c5ccccc5)ccc4c4c5sc6ccccc6c5ccc43)c2)cc1]

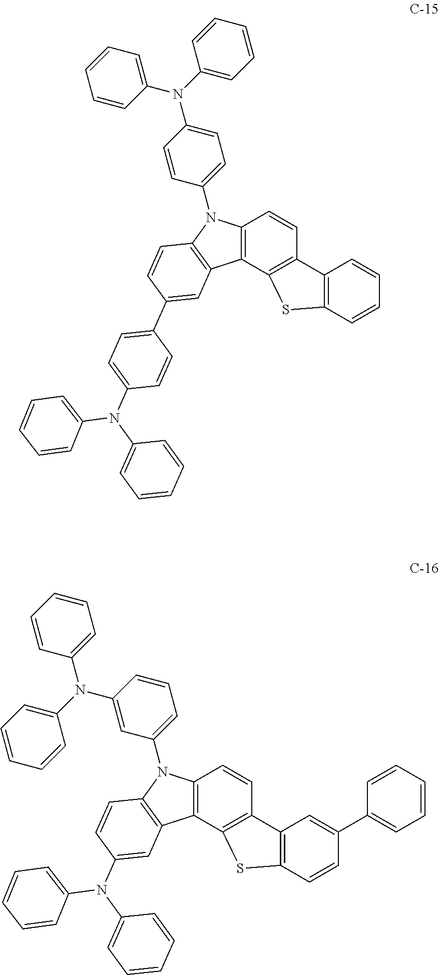 [SMILES: c1ccc(N(c2ccccc2)c2ccc(-c3ccc4c(c3)c3c5sc6ccccc6c5ccc3n4-c3ccc(N(c4ccccc4)c4ccccc4)cc3)cc2)cc1 ; c1ccc(-c2ccc3sc4c(ccc5c4c4cc(N(c6ccccc6)c6ccccc6)ccc4n5-c4cccc(N(c5ccccc5)c5ccccc5)c4)c3c2)cc1]

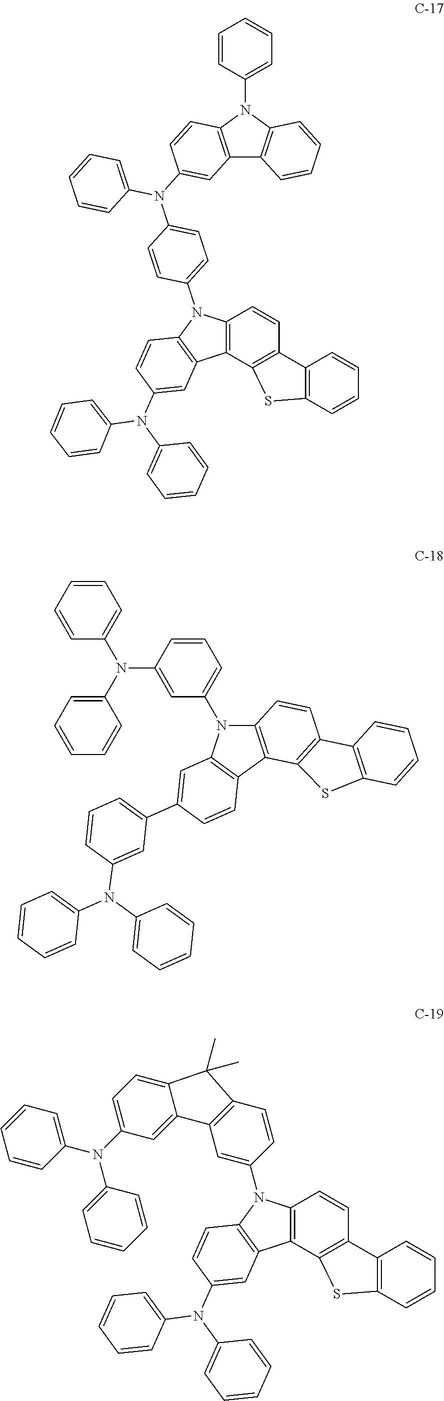 [SMILES: c1ccc(N(c2ccc(-n3c4ccc(N(c5ccccc5)c5ccccc5)cc4c4c5sc6ccccc6c5ccc43)cc2)c2ccc3c(c2)c2ccccc2n3-c2ccccc2)cc1 ; c1ccc(N(c2ccccc2)c2cccc(-c3ccc4c5c6sc7ccccc7c6ccc5n(-c5cccc(N(c6ccccc6)c6ccccc6)c5)c4c3)c2)cc1 ; CC1(C)c2ccc(N(c3ccccc3)c3ccccc3)cc2-c2cc(-n3c4ccc(N(c5ccccc5)c5ccccc5)cc4c4c5sc6ccccc6c5ccc43)ccc21]

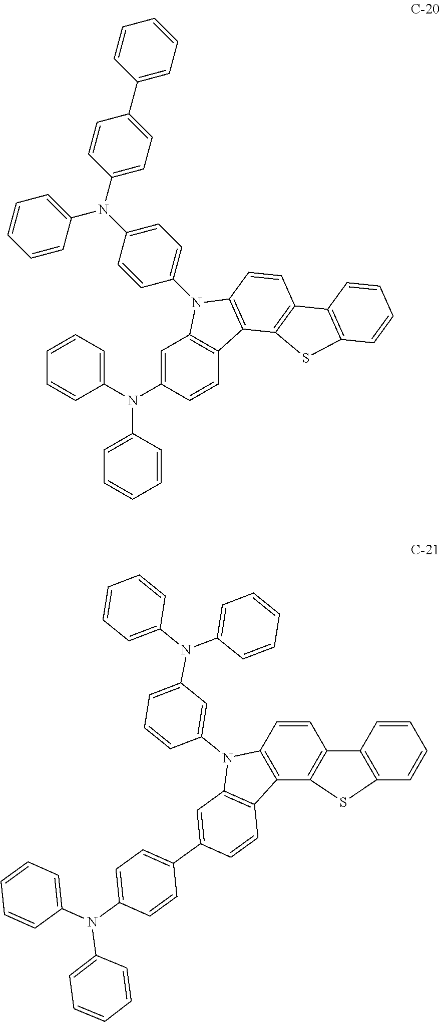 [SMILES: c1ccc(-c2ccc(N(c3ccccc3)c3ccc(-n4c5cc(N(c6ccccc6)c6ccccc6)ccc5c5c6sc7ccccc7c6ccc54)cc3)cc2)cc1 ; c1ccc(N(c2ccccc2)c2ccc(-c3ccc4c5c6sc7ccccc7c6ccc5n(-c5cccc(N(c6ccccc6)c6ccccc6)c5)c4c3)cc2)cc1]

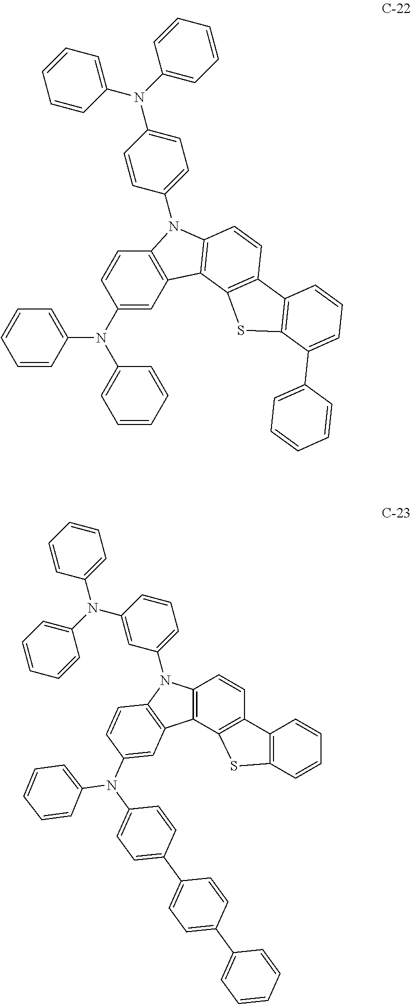 [SMILES: c1ccc(-c2cccc3c2sc2c3ccc3c2c2cc(N(c4ccccc4)c4ccccc4)ccc2n3-c2ccc(N(c3ccccc3)c3ccccc3)cc2)cc1 ; c1ccc(-c2ccc(-c3ccc(N(c4ccccc4)c4ccc5c(c4)c4c6sc7ccccc7c6ccc4n5-c4cccc(N(c5ccccc5)c5ccccc5)c4)cc3)cc2)cc1]

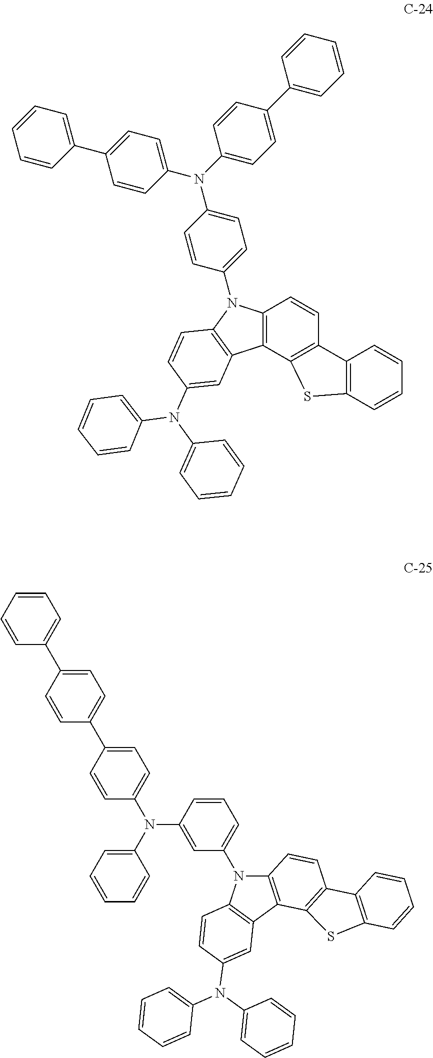 [SMILES: c1ccc(-c2ccc(N(c3ccc(-c4ccccc4)cc3)c3ccc(-n4c5ccc(N(c6ccccc6)c6ccccc6)cc5c5c6sc7ccccc7c6ccc54)cc3)cc2)cc1 ; c1ccc(-c2ccc(-c3ccc(N(c4ccccc4)c4cccc(-n5c6ccc(N(c7ccccc7)c7ccccc7)cc6c6c7sc8ccccc8c7ccc65)c4)cc3)cc2)cc1]

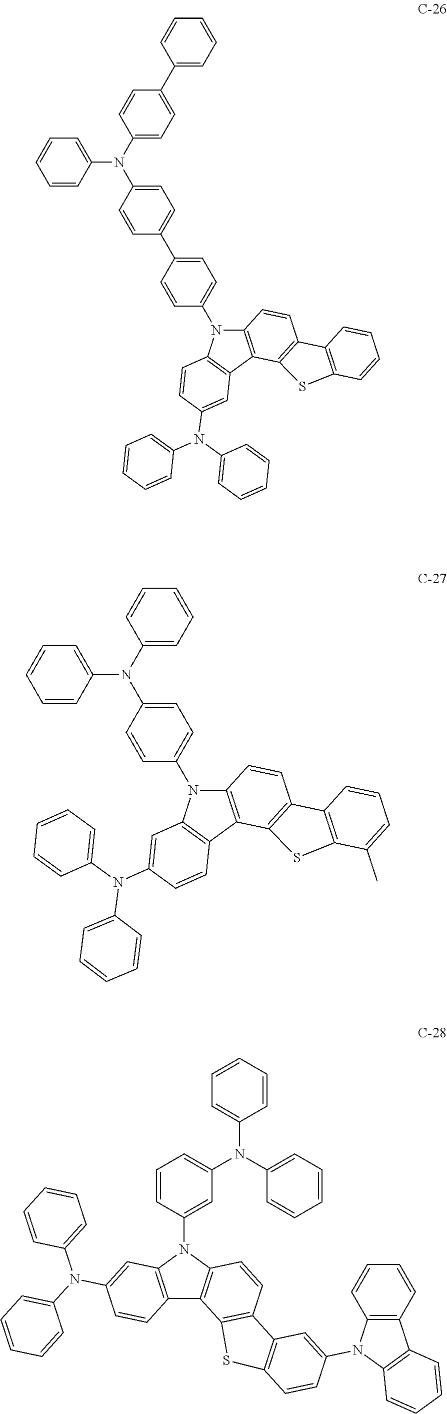 [SMILES: c1ccc(-c2ccc(N(c3ccccc3)c3ccc(-c4ccc(-n5c6ccc(N(c7ccccc7)c7ccccc7)cc6c6c7sc8ccccc8c7ccc65)cc4)cc3)cc2)cc1 ; Cc1cccc2c1sc1c2ccc2c1c1ccc(N(c3ccccc3)c3ccccc3)cc1n2-c1ccc(N(c2ccccc2)c2ccccc2)cc1 ; c1ccc(N(c2ccccc2)c2cccc(-n3c4cc(N(c5ccccc5)c5ccccc5)ccc4c4c5sc6ccc(-n7c8ccccc8c8ccccc87)cc6c5ccc43)c2)cc1]

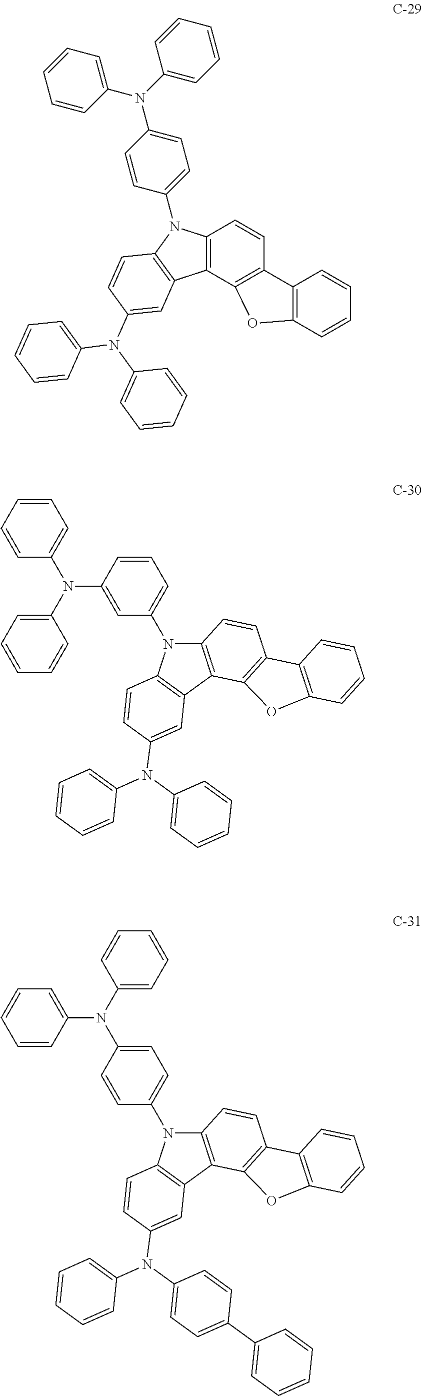 [SMILES: c1ccc(N(c2ccccc2)c2ccc(-n3c4ccc(N(c5ccccc5)c5ccccc5)cc4c4c5oc6ccccc6c5ccc43)cc2)cc1 ; c1ccc(N(c2ccccc2)c2cccc(-n3c4ccc(N(c5ccccc5)c5ccccc5)cc4c4c5oc6ccccc6c5ccc43)c2)cc1 ; c1ccc(-c2ccc(N(c3ccccc3)c3ccc4c(c3)c3c5oc6ccccc6c5ccc3n4-c3ccc(N(c4ccccc4)c4ccccc4)cc3)cc2)cc1]

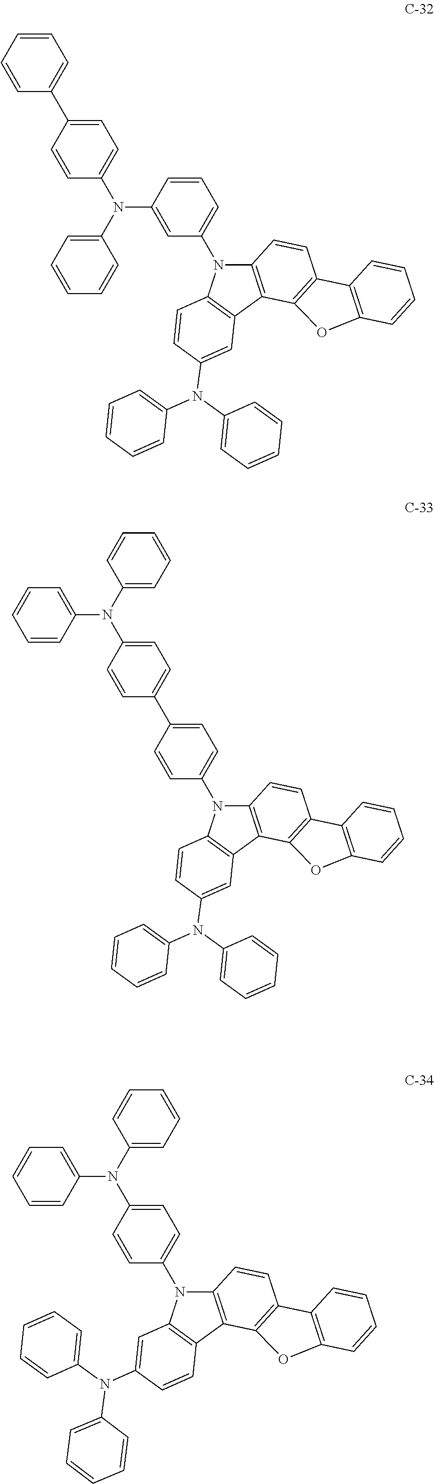 [SMILES: c1ccc(-c2ccc(N(c3ccccc3)c3cccc(-n4c5ccc(N(c6ccccc6)c6ccccc6)cc5c5c6oc7ccccc7c6ccc54)c3)cc2)cc1 ; c1ccc(N(c2ccccc2)c2ccc(-c3ccc(-n4c5ccc(N(c6ccccc6)c6ccccc6)cc5c5c6oc7ccccc7c6ccc54)cc3)cc2)cc1 ; c1ccc(N(c2ccccc2)c2ccc(-n3c4cc(N(c5ccccc5)c5ccccc5)ccc4c4c5oc6ccccc6c5ccc43)cc2)cc1]

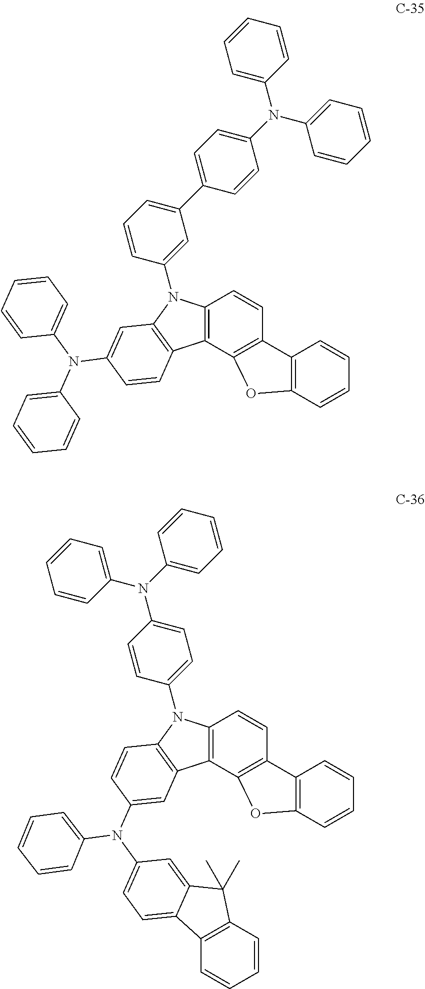 [SMILES: c1ccc(N(c2ccccc2)c2ccc(-c3cccc(-n4c5cc(N(c6ccccc6)c6ccccc6)ccc5c5c6oc7ccccc7c6ccc54)c3)cc2)cc1 ; CC1(C)c2ccccc2-c2ccc(N(c3ccccc3)c3ccc4c(c3)c3c5oc6ccccc6c5ccc3n4-c3ccc(N(c4ccccc4)c4ccccc4)cc3)cc21]

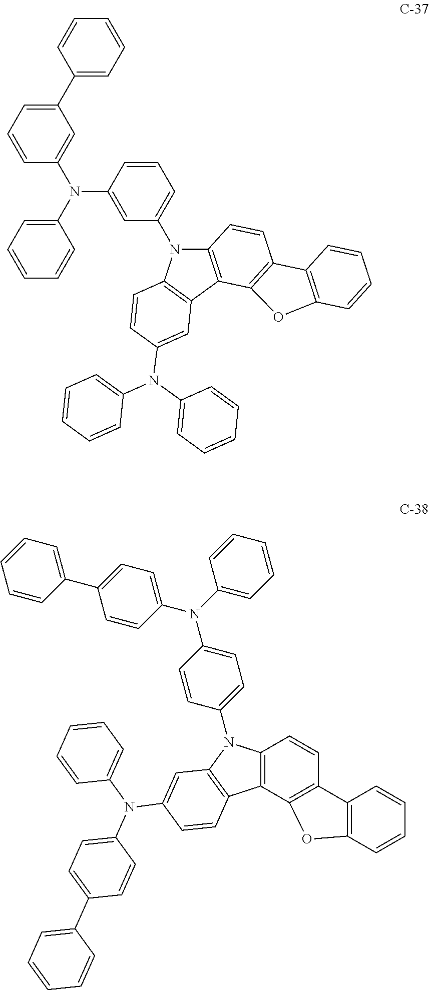 [SMILES: c1ccc(-c2cccc(N(c3ccccc3)c3cccc(-n4c5ccc(N(c6ccccc6)c6ccccc6)cc5c5c6oc7ccccc7c6ccc54)c3)c2)cc1 ; c1ccc(-c2ccc(N(c3ccccc3)c3ccc(-n4c5cc(N(c6ccccc6)c6ccc(-c7ccccc7)cc6)ccc5c5c6oc7ccccc7c6ccc54)cc3)cc2)cc1]

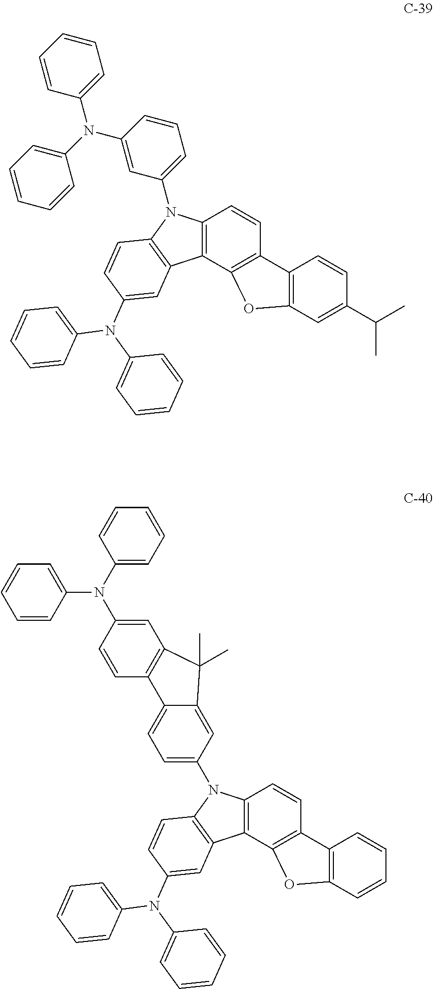 [SMILES: CC(C)c1ccc2c(c1)oc1c2ccc2c1c1cc(N(c3ccccc3)c3ccccc3)ccc1n2-c1cccc(N(c2ccccc2)c2ccccc2)c1 ; CC1(C)c2cc(N(c3ccccc3)c3ccccc3)ccc2-c2ccc(-n3c4ccc(N(c5ccccc5)c5ccccc5)cc4c4c5oc6ccccc6c5ccc43)cc21]

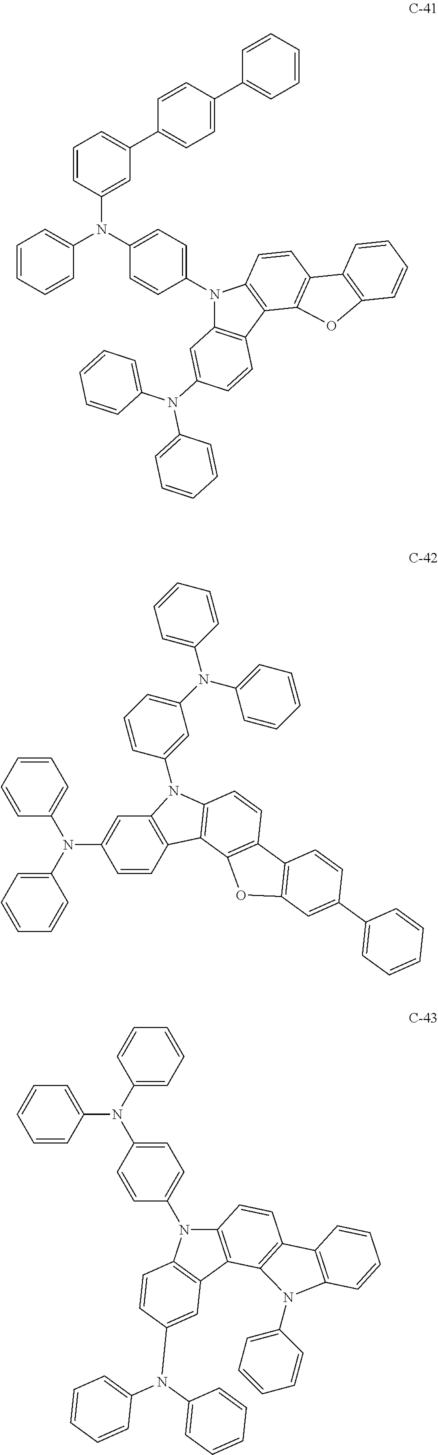 [SMILES: c1ccc(-c2ccc(-c3cccc(N(c4ccccc4)c4ccc(-n5c6cc(N(c7ccccc7)c7ccccc7)ccc6c6c7oc8ccccc8c7ccc65)cc4)c3)cc2)cc1 ; c1ccc(-c2ccc3c(c2)oc2c3ccc3c2c2ccc(N(c4ccccc4)c4ccccc4)cc2n3-c2cccc(N(c3ccccc3)c3ccccc3)c2)cc1 ; c1ccc(N(c2ccccc2)c2ccc(-n3c4ccc(N(c5ccccc5)c5ccccc5)cc4c4c3ccc3c5ccccc5n(-c5ccccc5)c34)cc2)cc1]

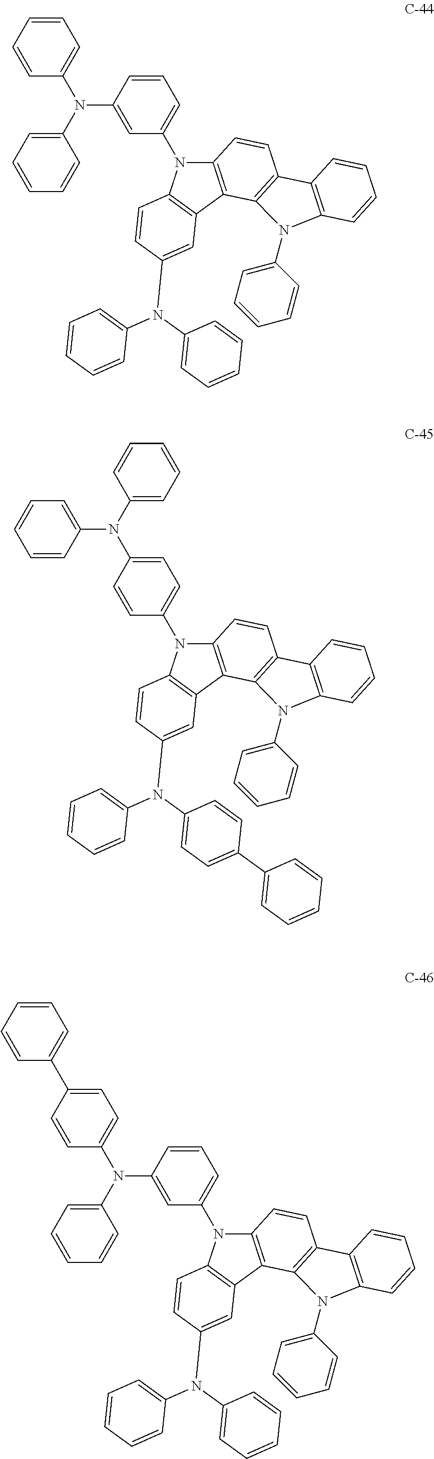 [SMILES: c1ccc(N(c2ccccc2)c2cccc(-n3c4ccc(N(c5ccccc5)c5ccccc5)cc4c4c3ccc3c5ccccc5n(-c5ccccc5)c34)c2)cc1 ; c1ccc(-c2ccc(N(c3ccccc3)c3ccc4c(c3)c3c(ccc5c6ccccc6n(-c6ccccc6)c53)n4-c3ccc(N(c4ccccc4)c4ccccc4)cc3)cc2)cc1 ; c1ccc(-c2ccc(N(c3ccccc3)c3cccc(-n4c5ccc(N(c6ccccc6)c6ccccc6)cc5c5c4ccc4c6ccccc6n(-c6ccccc6)c45)c3)cc2)cc1]

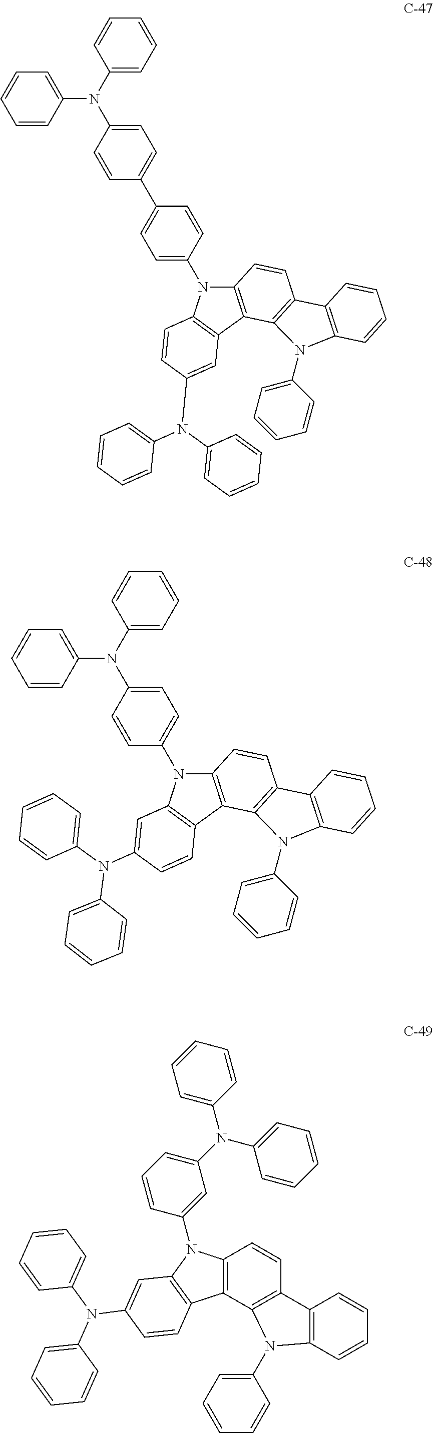 [SMILES: c1ccc(N(c2ccccc2)c2ccc(-c3ccc(-n4c5ccc(N(c6ccccc6)c6ccccc6)cc5c5c4ccc4c6ccccc6n(-c6ccccc6)c45)cc3)cc2)cc1 ; c1ccc(N(c2ccccc2)c2ccc(-n3c4cc(N(c5ccccc5)c5ccccc5)ccc4c4c3ccc3c5ccccc5n(-c5ccccc5)c34)cc2)cc1 ; c1ccc(N(c2ccccc2)c2cccc(-n3c4cc(N(c5ccccc5)c5ccccc5)ccc4c4c3ccc3c5ccccc5n(-c5ccccc5)c34)c2)cc1]

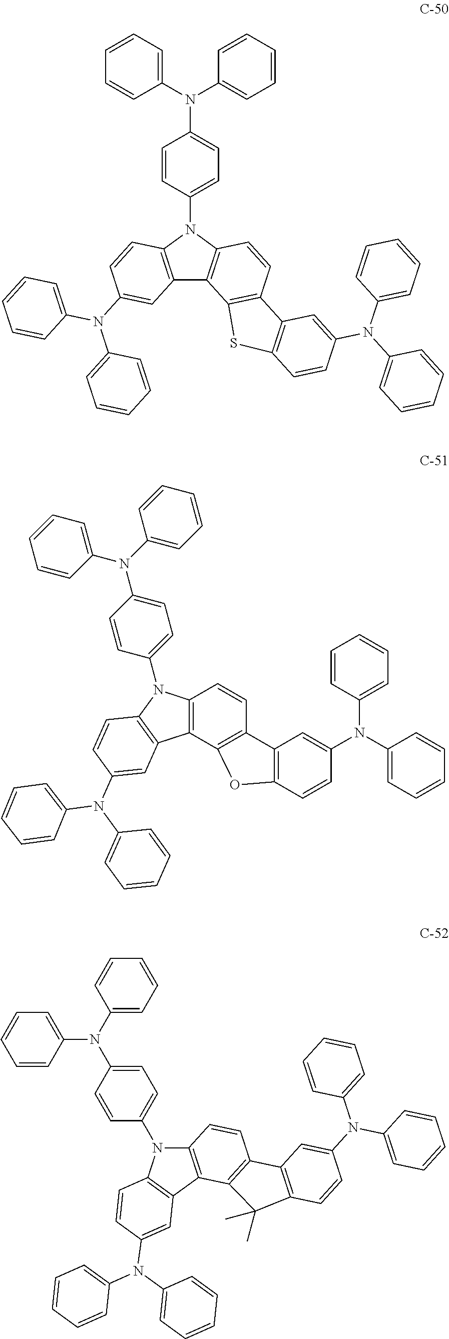 [SMILES: c1ccc(N(c2ccccc2)c2ccc(-n3c4ccc(N(c5ccccc5)c5ccccc5)cc4c4c5sc6ccc(N(c7ccccc7)c7ccccc7)cc6c5ccc43)cc2)cc1 ; c1ccc(N(c2ccccc2)c2ccc(-n3c4ccc(N(c5ccccc5)c5ccccc5)cc4c4c5oc6ccc(N(c7ccccc7)c7ccccc7)cc6c5ccc43)cc2)cc1 ; CC1(C)c2ccc(N(c3ccccc3)c3ccccc3)cc2-c2ccc3c(c21)c1cc(N(c2ccccc2)c2ccccc2)ccc1n3-c1ccc(N(c2ccccc2)c2ccccc2)cc1]

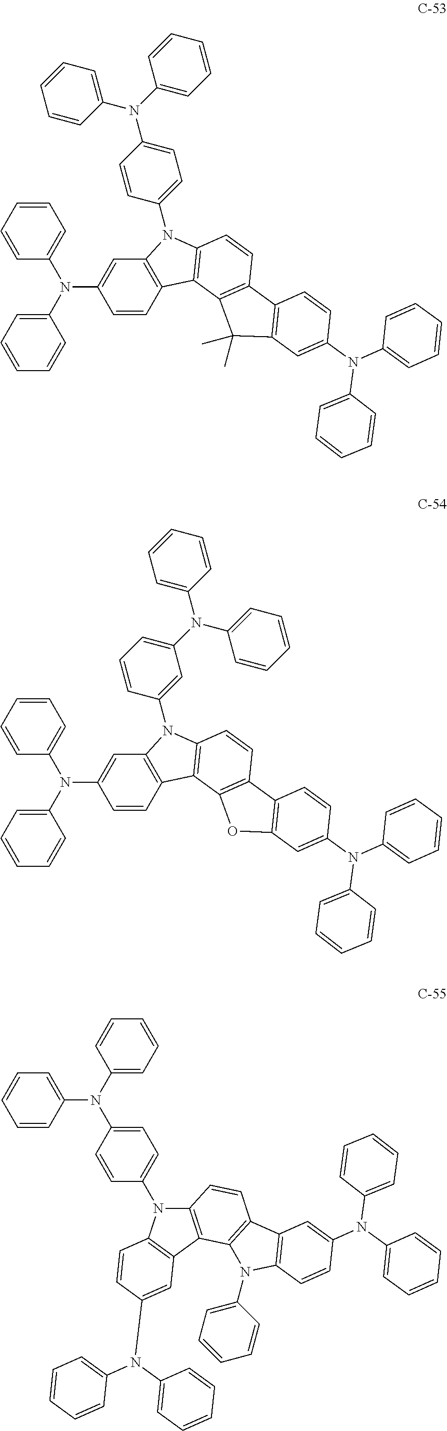 [SMILES: CC1(C)c2cc(N(c3ccccc3)c3ccccc3)ccc2-c2ccc3c(c21)c1ccc(N(c2ccccc2)c2ccccc2)cc1n3-c1ccc(N(c2ccccc2)c2ccccc2)cc1 ; c1ccc(N(c2ccccc2)c2cccc(-n3c4cc(N(c5ccccc5)c5ccccc5)ccc4c4c5oc6cc(N(c7ccccc7)c7ccccc7)ccc6c5ccc43)c2)cc1 ; c1ccc(N(c2ccccc2)c2ccc(-n3c4ccc(N(c5ccccc5)c5ccccc5)cc4c4c3ccc3c5cc(N(c6ccccc6)c6ccccc6)ccc5n(-c5ccccc5)c34)cc2)cc1]

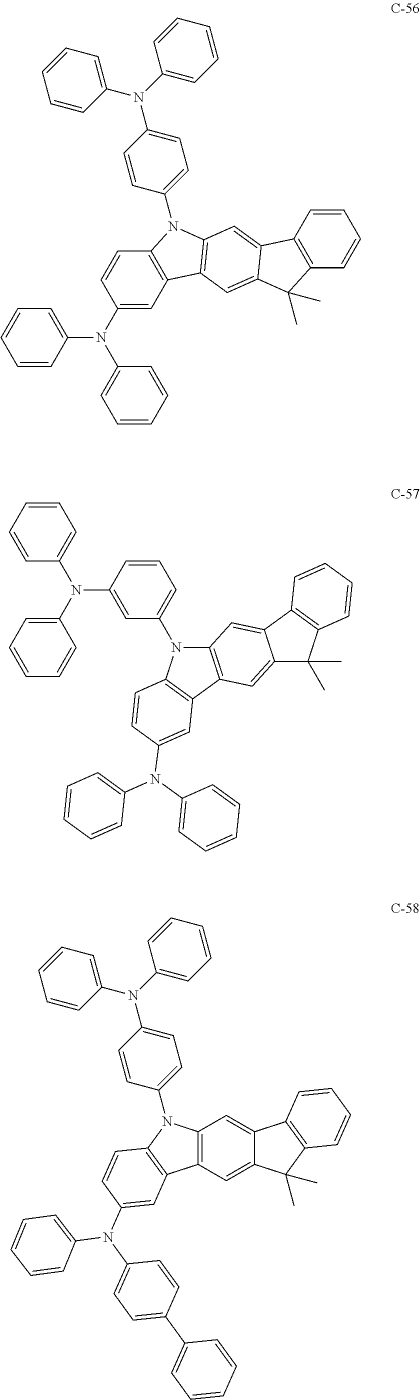 [SMILES: CC1(C)c2ccccc2-c2cc3c(cc21)c1cc(N(c2ccccc2)c2ccccc2)ccc1n3-c1ccc(N(c2ccccc2)c2ccccc2)cc1 ; CC1(C)c2ccccc2-c2cc3c(cc21)c1cc(N(c2ccccc2)c2ccccc2)ccc1n3-c1cccc(N(c2ccccc2)c2ccccc2)c1 ; CC1(C)c2ccccc2-c2cc3c(cc21)c1cc(N(c2ccccc2)c2ccc(-c4ccccc4)cc2)ccc1n3-c1ccc(N(c2ccccc2)c2ccccc2)cc1]

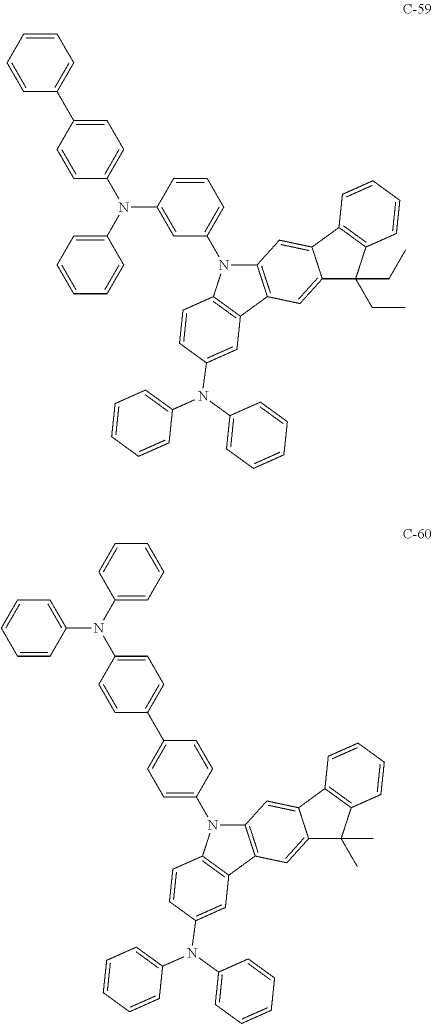 [SMILES: CCC1(CC)c2ccccc2-c2cc3c(cc21)c1cc(N(c2ccccc2)c2ccccc2)ccc1n3-c1cccc(N(c2ccccc2)c2ccc(-c3ccccc3)cc2)c1 ; CC1(C)c2ccccc2-c2cc3c(cc21)c1cc(N(c2ccccc2)c2ccccc2)ccc1n3-c1ccc(-c2ccc(N(c3ccccc3)c3ccccc3)cc2)cc1]

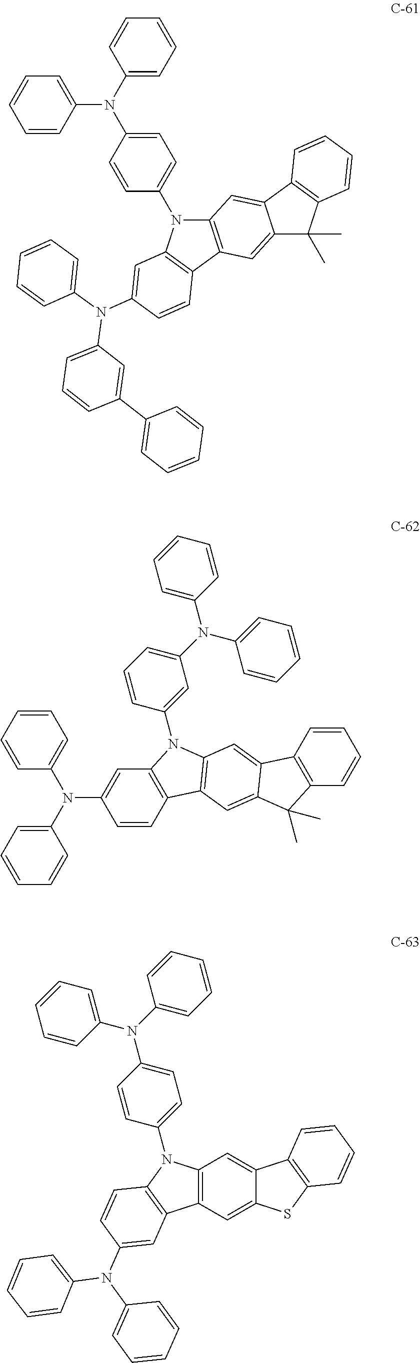 [SMILES: CC1(C)c2ccccc2-c2cc3c(cc21)c1ccc(N(c2ccccc2)c2cccc(-c4ccccc4)c2)cc1n3-c1ccc(N(c2ccccc2)c2ccccc2)cc1 ; CC1(C)c2ccccc2-c2cc3c(cc21)c1ccc(N(c2ccccc2)c2ccccc2)cc1n3-c1cccc(N(c2ccccc2)c2ccccc2)c1 ; c1ccc(N(c2ccccc2)c2ccc(-n3c4ccc(N(c5ccccc5)c5ccccc5)cc4c4cc5sc6ccccc6c5cc43)cc2)cc1]

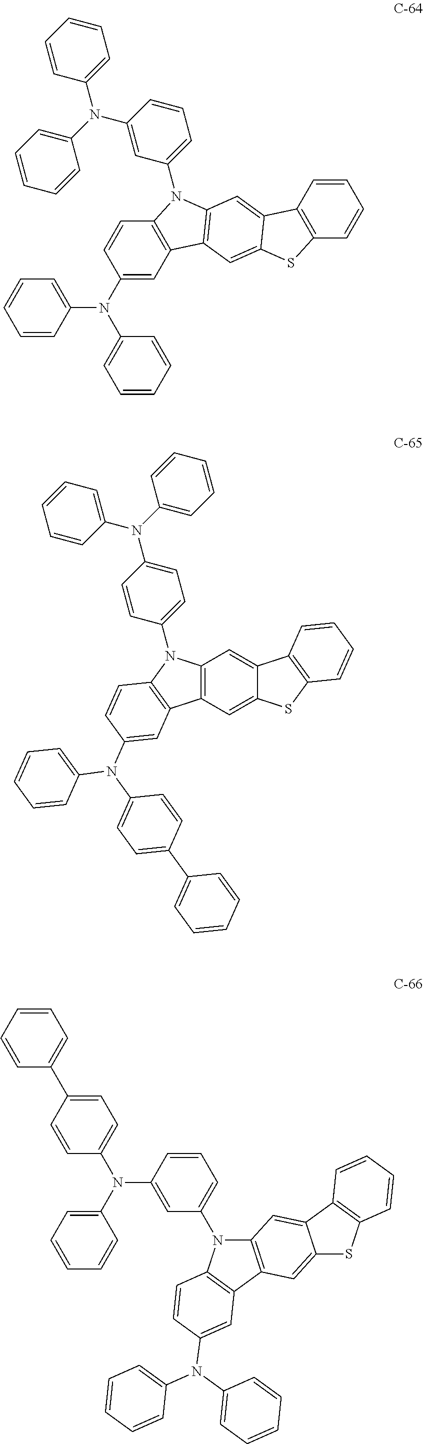 [SMILES: c1ccc(N(c2ccccc2)c2cccc(-n3c4ccc(N(c5ccccc5)c5ccccc5)cc4c4cc5sc6ccccc6c5cc43)c2)cc1 ; c1ccc(-c2ccc(N(c3ccccc3)c3ccc4c(c3)c3cc5sc6ccccc6c5cc3n4-c3ccc(N(c4ccccc4)c4ccccc4)cc3)cc2)cc1 ; c1ccc(-c2ccc(N(c3ccccc3)c3cccc(-n4c5ccc(N(c6ccccc6)c6ccccc6)cc5c5cc6sc7ccccc7c6cc54)c3)cc2)cc1]

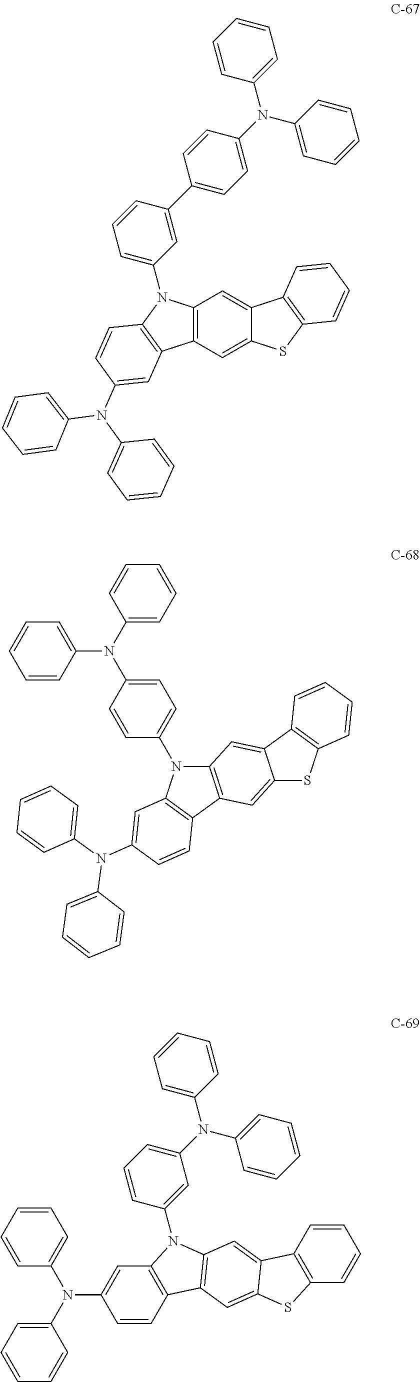 [SMILES: c1ccc(N(c2ccccc2)c2ccc(-c3cccc(-n4c5ccc(N(c6ccccc6)c6ccccc6)cc5c5cc6sc7ccccc7c6cc54)c3)cc2)cc1 ; c1ccc(N(c2ccccc2)c2ccc(-n3c4cc(N(c5ccccc5)c5ccccc5)ccc4c4cc5sc6ccccc6c5cc43)cc2)cc1 ; c1ccc(N(c2ccccc2)c2cccc(-n3c4cc(N(c5ccccc5)c5ccccc5)ccc4c4cc5sc6ccccc6c5cc43)c2)cc1]

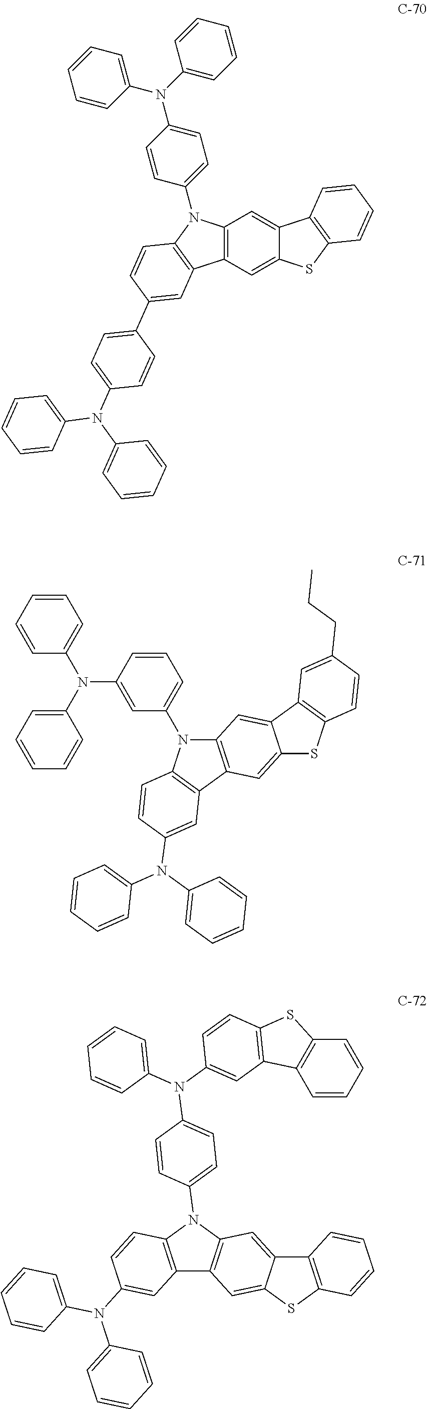 [SMILES: c1ccc(N(c2ccccc2)c2ccc(-c3ccc4c(c3)c3cc5sc6ccccc6c5cc3n4-c3ccc(N(c4ccccc4)c4ccccc4)cc3)cc2)cc1 ; CCCc1ccc2sc3cc4c5cc(N(c6ccccc6)c6ccccc6)ccc5n(-c5cccc(N(c6ccccc6)c6ccccc6)c5)c4cc3c2c1 ; c1ccc(N(c2ccc(-n3c4ccc(N(c5ccccc5)c5ccccc5)cc4c4cc5sc6ccccc6c5cc43)cc2)c2ccc3sc4ccccc4c3c2)cc1]

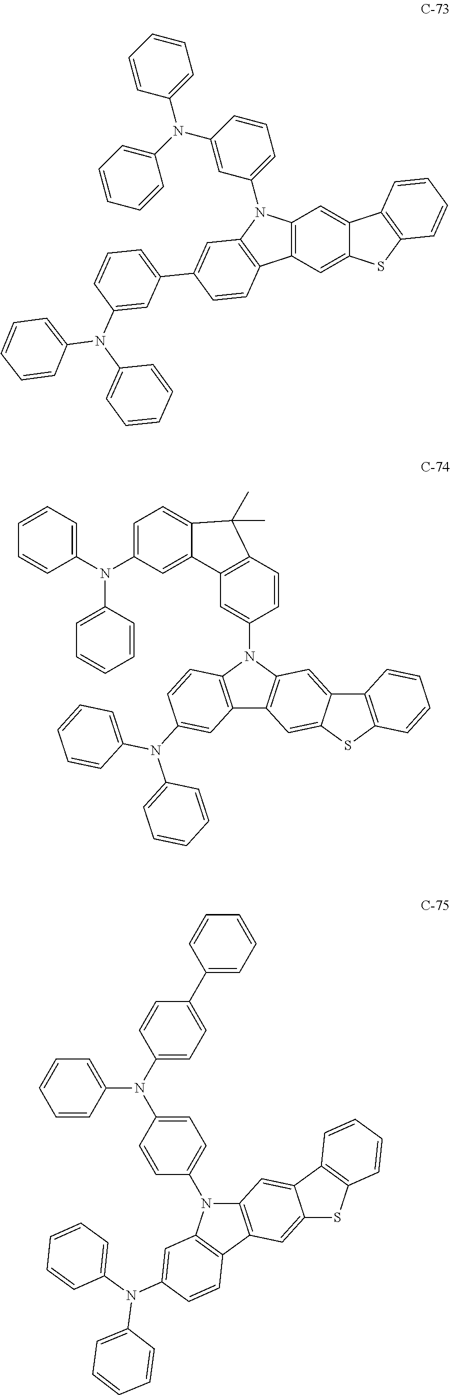 [SMILES: c1ccc(N(c2ccccc2)c2cccc(-c3ccc4c5cc6sc7ccccc7c6cc5n(-c5cccc(N(c6ccccc6)c6ccccc6)c5)c4c3)c2)cc1 ; CC1(C)c2ccc(N(c3ccccc3)c3ccccc3)cc2-c2cc(-n3c4ccc(N(c5ccccc5)c5ccccc5)cc4c4cc5sc6ccccc6c5cc43)ccc21 ; c1ccc(-c2ccc(N(c3ccccc3)c3ccc(-n4c5cc(N(c6ccccc6)c6ccccc6)ccc5c5cc6sc7ccccc7c6cc54)cc3)cc2)cc1]

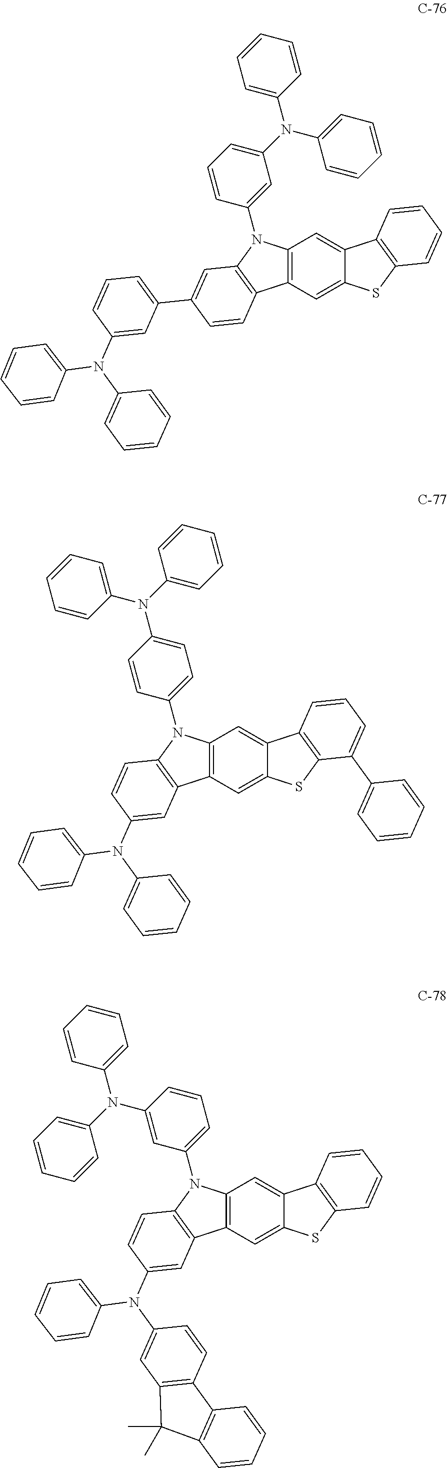 [SMILES: c1ccc(N(c2ccccc2)c2cccc(-c3ccc4c5cc6sc7ccccc7c6cc5n(-c5cccc(N(c6ccccc6)c6ccccc6)c5)c4c3)c2)cc1 ; c1ccc(-c2cccc3c2sc2cc4c5cc(N(c6ccccc6)c6ccccc6)ccc5n(-c5ccc(N(c6ccccc6)c6ccccc6)cc5)c4cc23)cc1 ; CC1(C)c2ccccc2-c2ccc(N(c3ccccc3)c3ccc4c(c3)c3cc5sc6ccccc6c5cc3n4-c3cccc(N(c4ccccc4)c4ccccc4)c3)cc21]

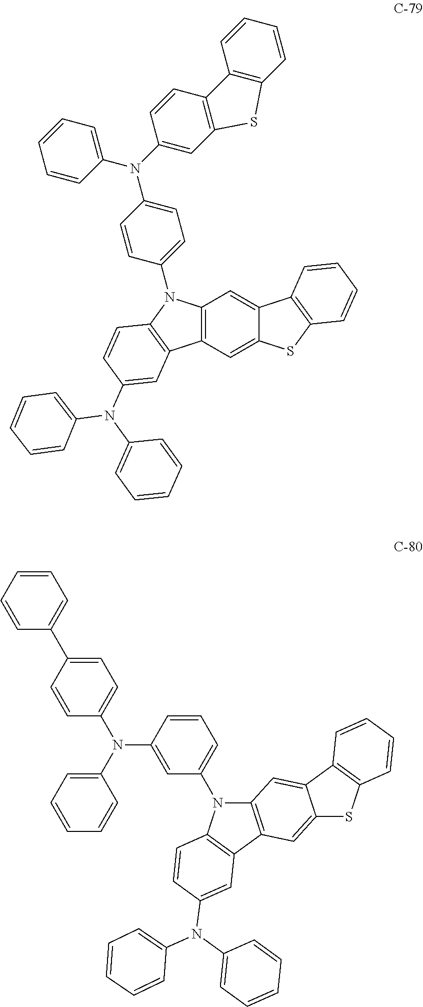 [SMILES: c1ccc(N(c2ccc(-n3c4ccc(N(c5ccccc5)c5ccccc5)cc4c4cc5sc6ccccc6c5cc43)cc2)c2ccc3c(c2)sc2ccccc23)cc1 ; c1ccc(-c2ccc(N(c3ccccc3)c3cccc(-n4c5ccc(N(c6ccccc6)c6ccccc6)cc5c5cc6sc7ccccc7c6cc54)c3)cc2)cc1]

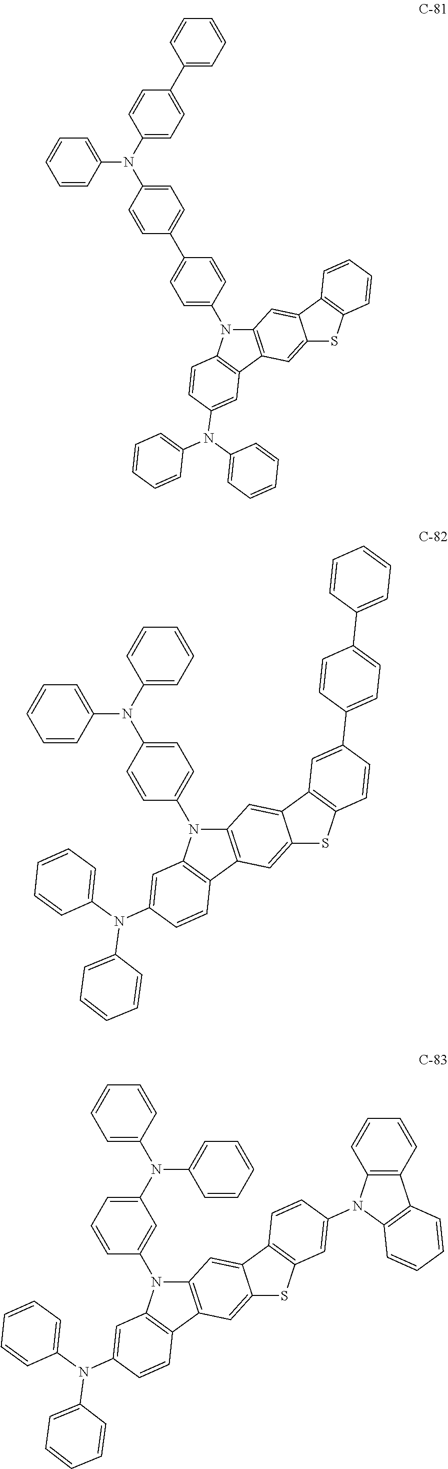 [SMILES: c1ccc(-c2ccc(N(c3ccccc3)c3ccc(-c4ccc(-n5c6ccc(N(c7ccccc7)c7ccccc7)cc6c6cc7sc8ccccc8c7cc65)cc4)cc3)cc2)cc1 ; c1ccc(-c2ccc(-c3ccc4sc5cc6c7ccc(N(c8ccccc8)c8ccccc8)cc7n(-c7ccc(N(c8ccccc8)c8ccccc8)cc7)c6cc5c4c3)cc2)cc1 ; c1ccc(N(c2ccccc2)c2cccc(-n3c4cc(N(c5ccccc5)c5ccccc5)ccc4c4cc5sc6cc(-n7c8ccccc8c8ccccc87)ccc6c5cc43)c2)cc1]

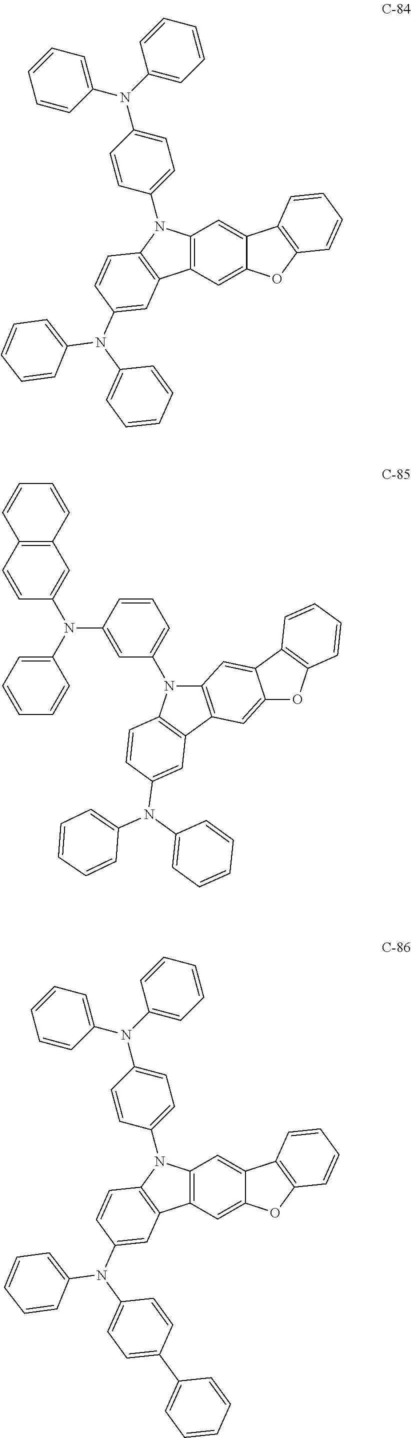 [SMILES: c1ccc(N(c2ccccc2)c2ccc(-n3c4ccc(N(c5ccccc5)c5ccccc5)cc4c4cc5oc6ccccc6c5cc43)cc2)cc1 ; c1ccc(N(c2cccc(-n3c4ccc(N(c5ccccc5)c5ccccc5)cc4c4cc5oc6ccccc6c5cc43)c2)c2ccc3ccccc3c2)cc1 ; c1ccc(-c2ccc(N(c3ccccc3)c3ccc4c(c3)c3cc5oc6ccccc6c5cc3n4-c3ccc(N(c4ccccc4)c4ccccc4)cc3)cc2)cc1]

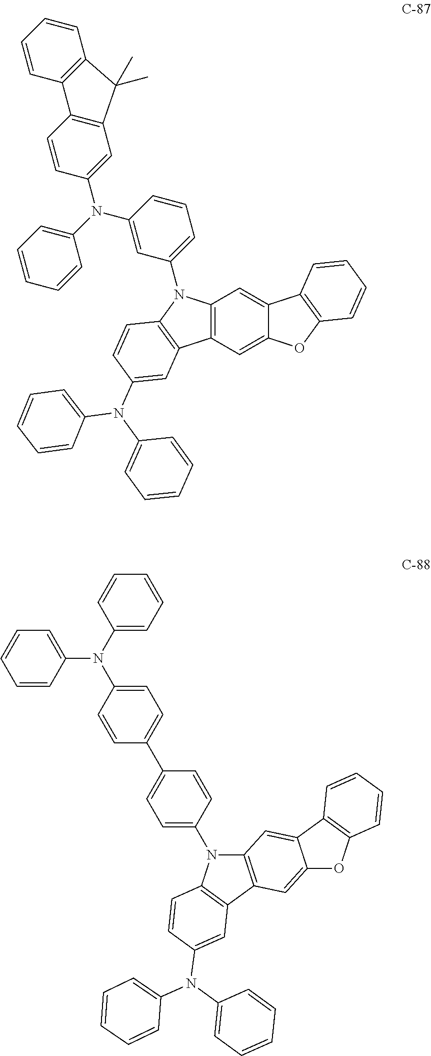 [SMILES: CC1(C)c2ccccc2-c2ccc(N(c3ccccc3)c3cccc(-n4c5ccc(N(c6ccccc6)c6ccccc6)cc5c5cc6oc7ccccc7c6cc54)c3)cc21 ; c1ccc(N(c2ccccc2)c2ccc(-c3ccc(-n4c5ccc(N(c6ccccc6)c6ccccc6)cc5c5cc6oc7ccccc7c6cc54)cc3)cc2)cc1]

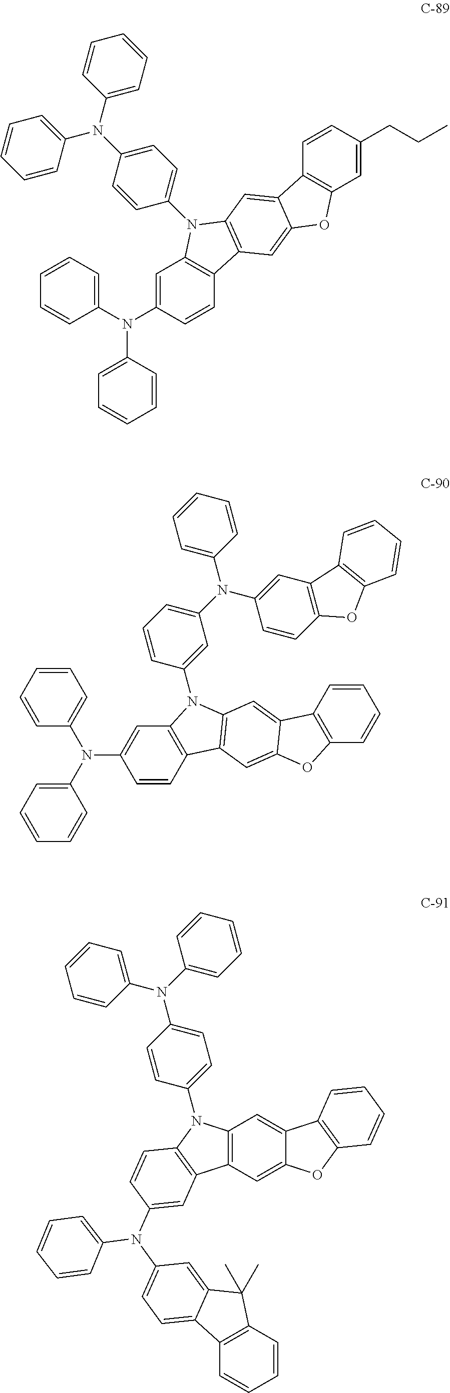 [SMILES: CCCc1ccc2c(c1)oc1cc3c4ccc(N(c5ccccc5)c5ccccc5)cc4n(-c4ccc(N(c5ccccc5)c5ccccc5)cc4)c3cc12 ; c1ccc(N(c2cccc(-n3c4cc(N(c5ccccc5)c5ccccc5)ccc4c4cc5oc6ccccc6c5cc43)c2)c2ccc3oc4ccccc4c3c2)cc1 ; CC1(C)c2ccccc2-c2ccc(N(c3ccccc3)c3ccc4c(c3)c3cc5oc6ccccc6c5cc3n4-c3ccc(N(c4ccccc4)c4ccccc4)cc3)cc21]

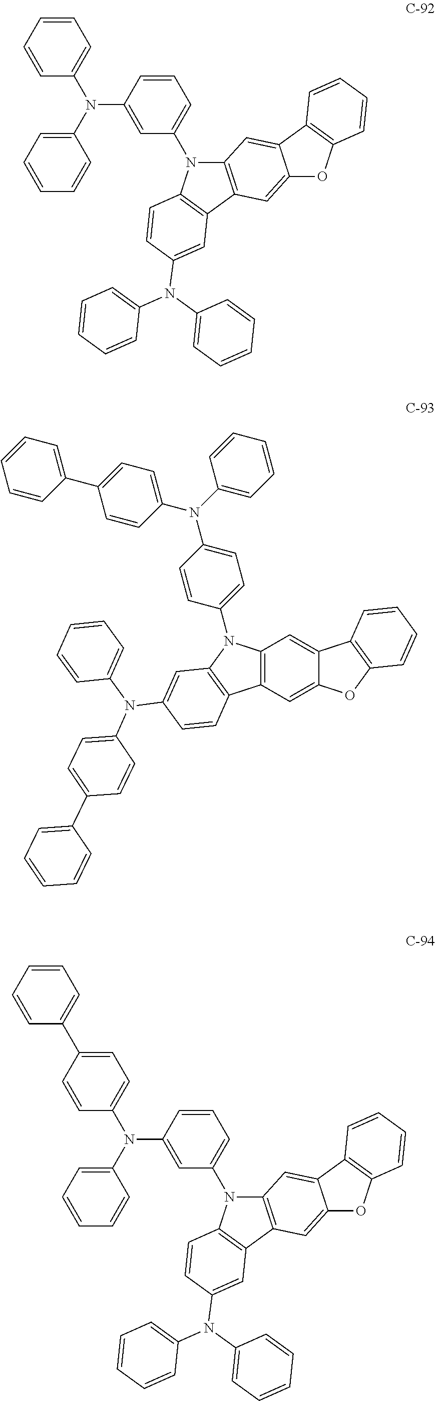 [SMILES: c1ccc(N(c2ccccc2)c2cccc(-n3c4ccc(N(c5ccccc5)c5ccccc5)cc4c4cc5oc6ccccc6c5cc43)c2)cc1 ; c1ccc(-c2ccc(N(c3ccccc3)c3ccc(-n4c5cc(N(c6ccccc6)c6ccc(-c7ccccc7)cc6)ccc5c5cc6oc7ccccc7c6cc54)cc3)cc2)cc1 ; c1ccc(-c2ccc(N(c3ccccc3)c3cccc(-n4c5ccc(N(c6ccccc6)c6ccccc6)cc5c5cc6oc7ccccc7c6cc54)c3)cc2)cc1]

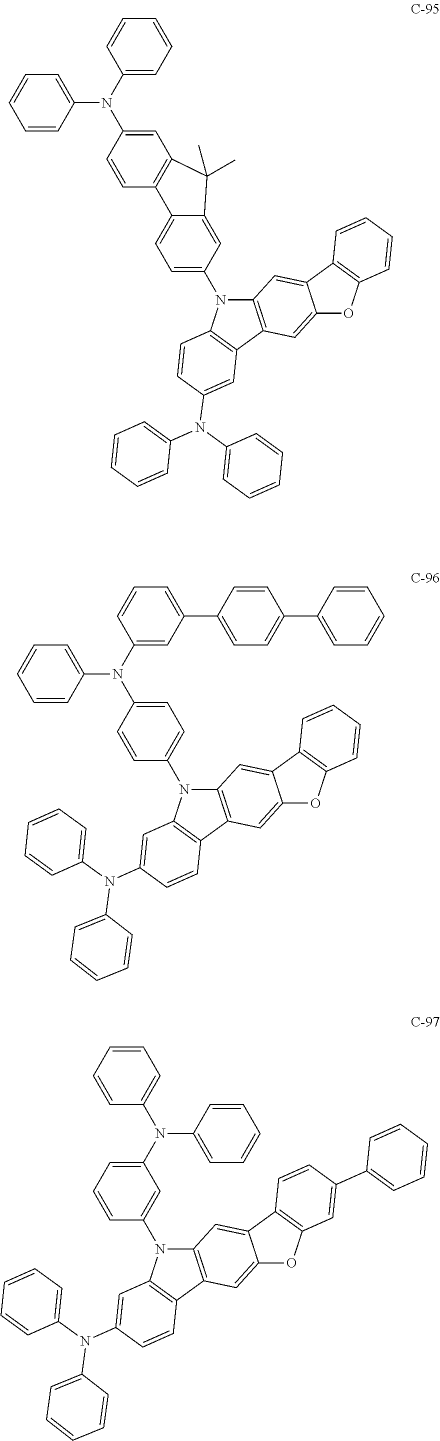 [SMILES: CC1(C)c2cc(N(c3ccccc3)c3ccccc3)ccc2-c2ccc(-n3c4ccc(N(c5ccccc5)c5ccccc5)cc4c4cc5oc6ccccc6c5cc43)cc21 ; c1ccc(-c2ccc(-c3cccc(N(c4ccccc4)c4ccc(-n5c6cc(N(c7ccccc7)c7ccccc7)ccc6c6cc7oc8ccccc8c7cc65)cc4)c3)cc2)cc1 ; c1ccc(-c2ccc3c(c2)oc2cc4c5ccc(N(c6ccccc6)c6ccccc6)cc5n(-c5cccc(N(c6ccccc6)c6ccccc6)c5)c4cc23)cc1]

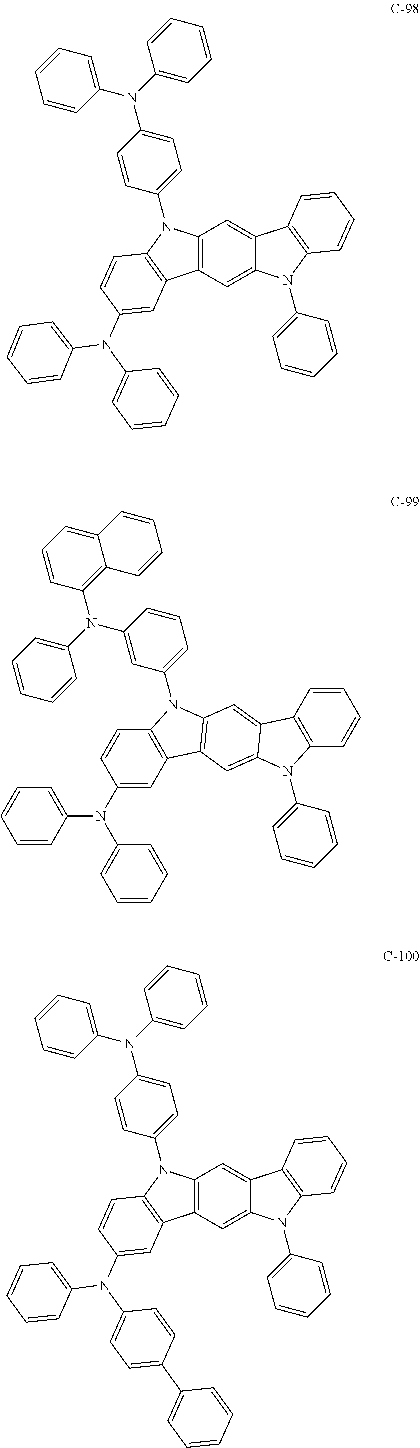 [SMILES: c1ccc(N(c2ccccc2)c2ccc(-n3c4ccc(N(c5ccccc5)c5ccccc5)cc4c4cc5c(cc43)c3ccccc3n5-c3ccccc3)cc2)cc1 ; c1ccc(N(c2ccccc2)c2ccc3c(c2)c2cc4c(cc2n3-c2cccc(N(c3ccccc3)c3cccc5ccccc35)c2)c2ccccc2n4-c2ccccc2)cc1 ; c1ccc(-c2ccc(N(c3ccccc3)c3ccc4c(c3)c3cc5c(cc3n4-c3ccc(N(c4ccccc4)c4ccccc4)cc3)c3ccccc3n5-c3ccccc3)cc2)cc1]

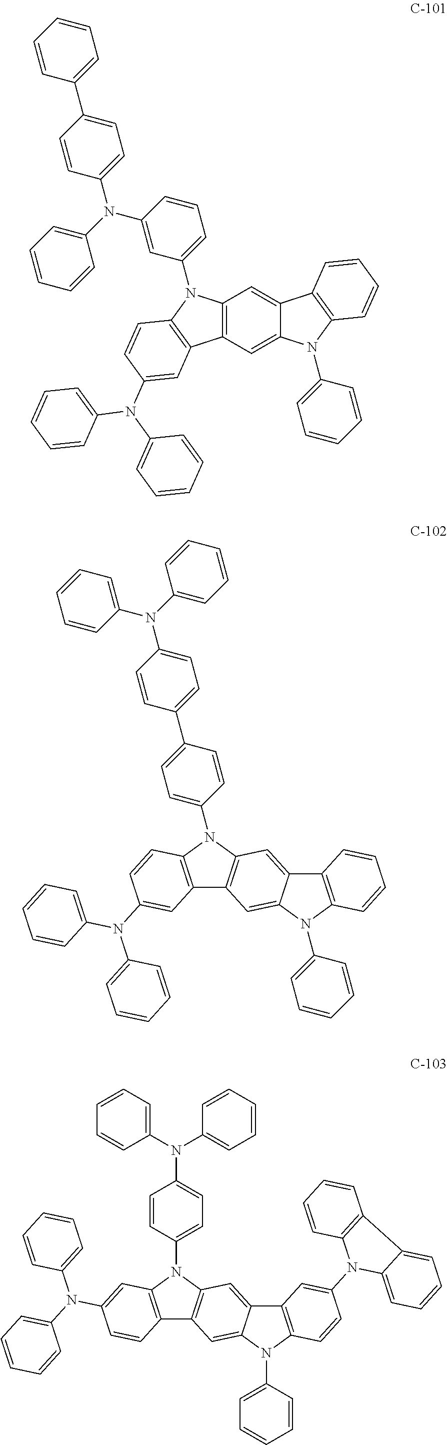 [SMILES: c1ccc(-c2ccc(N(c3ccccc3)c3cccc(-n4c5ccc(N(c6ccccc6)c6ccccc6)cc5c5cc6c(cc54)c4ccccc4n6-c4ccccc4)c3)cc2)cc1 ; c1ccc(N(c2ccccc2)c2ccc(-c3ccc(-n4c5ccc(N(c6ccccc6)c6ccccc6)cc5c5cc6c(cc54)c4ccccc4n6-c4ccccc4)cc3)cc2)cc1 ; c1ccc(N(c2ccccc2)c2ccc(-n3c4cc(N(c5ccccc5)c5ccccc5)ccc4c4cc5c(cc43)c3cc(-n4c6ccccc6c6ccccc64)ccc3n5-c3ccccc3)cc2)cc1]

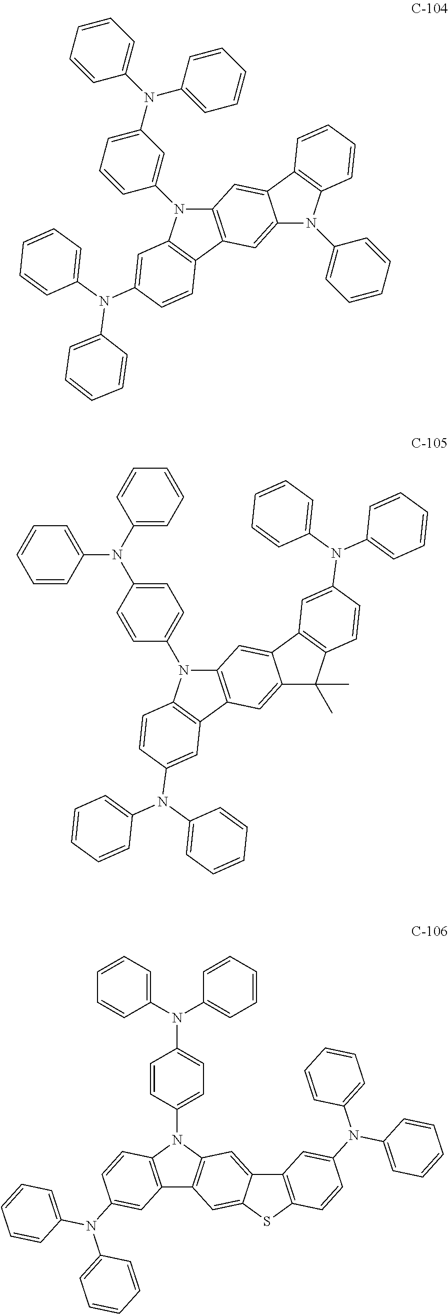 [SMILES: c1ccc(N(c2ccccc2)c2cccc(-n3c4cc(N(c5ccccc5)c5ccccc5)ccc4c4cc5c(cc43)c3ccccc3n5-c3ccccc3)c2)cc1 ; CC1(C)c2ccc(N(c3ccccc3)c3ccccc3)cc2-c2cc3c(cc21)c1cc(N(c2ccccc2)c2ccccc2)ccc1n3-c1ccc(N(c2ccccc2)c2ccccc2)cc1 ; c1ccc(N(c2ccccc2)c2ccc(-n3c4ccc(N(c5ccccc5)c5ccccc5)cc4c4cc5sc6ccc(N(c7ccccc7)c7ccccc7)cc6c5cc43)cc2)cc1]

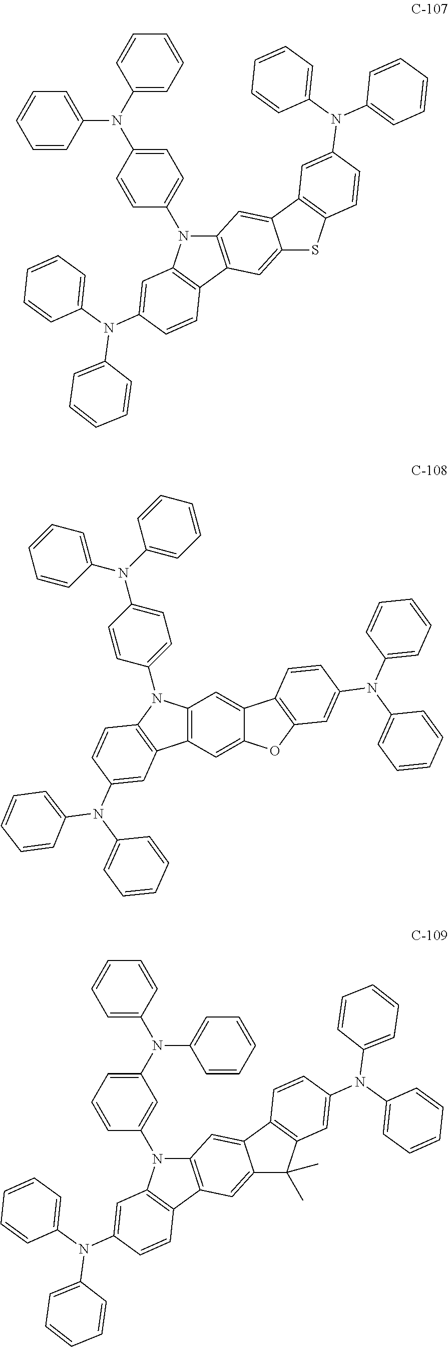 [SMILES: c1ccc(N(c2ccccc2)c2ccc(-n3c4cc(N(c5ccccc5)c5ccccc5)ccc4c4cc5sc6ccc(N(c7ccccc7)c7ccccc7)cc6c5cc43)cc2)cc1 ; c1ccc(N(c2ccccc2)c2ccc(-n3c4ccc(N(c5ccccc5)c5ccccc5)cc4c4cc5oc6cc(N(c7ccccc7)c7ccccc7)ccc6c5cc43)cc2)cc1 ; CC1(C)c2cc(N(c3ccccc3)c3ccccc3)ccc2-c2cc3c(cc21)c1ccc(N(c2ccccc2)c2ccccc2)cc1n3-c1cccc(N(c2ccccc2)c2ccccc2)c1]

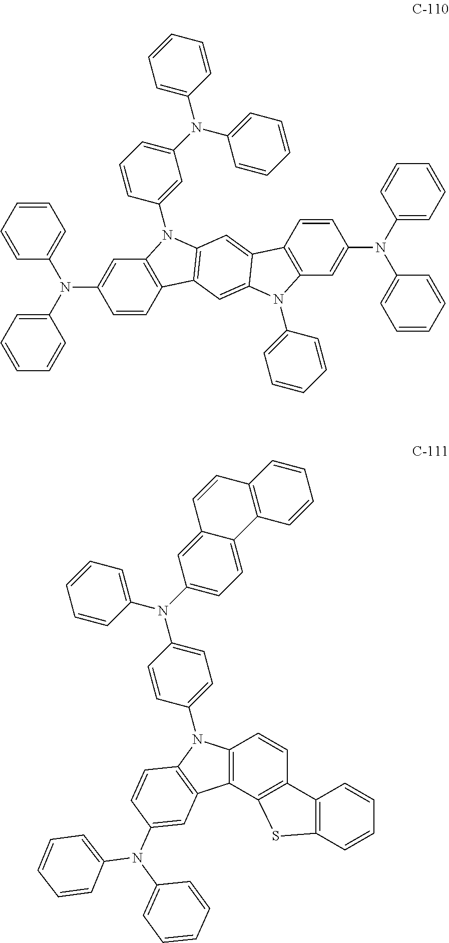 [SMILES: c1ccc(N(c2ccccc2)c2cccc(-n3c4cc(N(c5ccccc5)c5ccccc5)ccc4c4cc5c(cc43)c3ccc(N(c4ccccc4)c4ccccc4)cc3n5-c3ccccc3)c2)cc1 ; c1ccc(N(c2ccc(-n3c4ccc(N(c5ccccc5)c5ccccc5)cc4c4c5sc6ccccc6c5ccc43)cc2)c2ccc3c(ccc4ccccc43)c2)cc1]

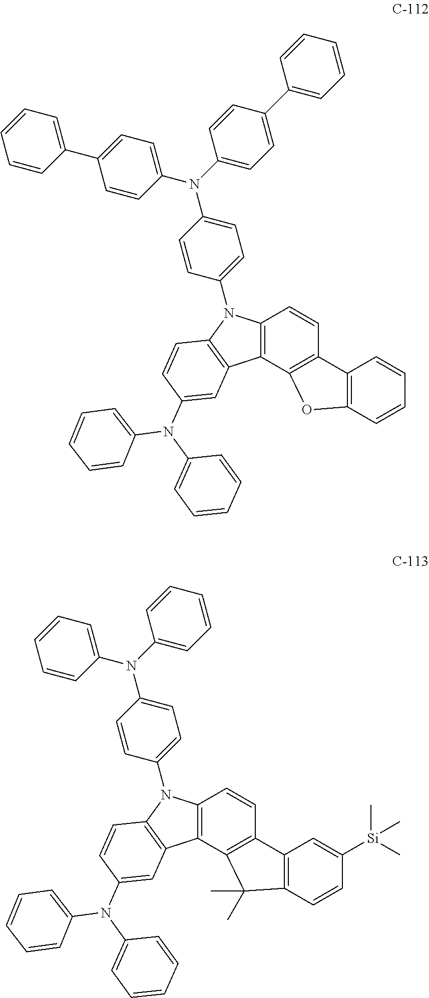 [SMILES: c1ccc(-c2ccc(N(c3ccc(-c4ccccc4)cc3)c3ccc(-n4c5ccc(N(c6ccccc6)c6ccccc6)cc5c5c6oc7ccccc7c6ccc54)cc3)cc2)cc1 ; CC1(C)c2ccc([Si](C)(C)C)cc2-c2ccc3c(c21)c1cc(N(c2ccccc2)c2ccccc2)ccc1n3-c1ccc(N(c2ccccc2)c2ccccc2)cc1]

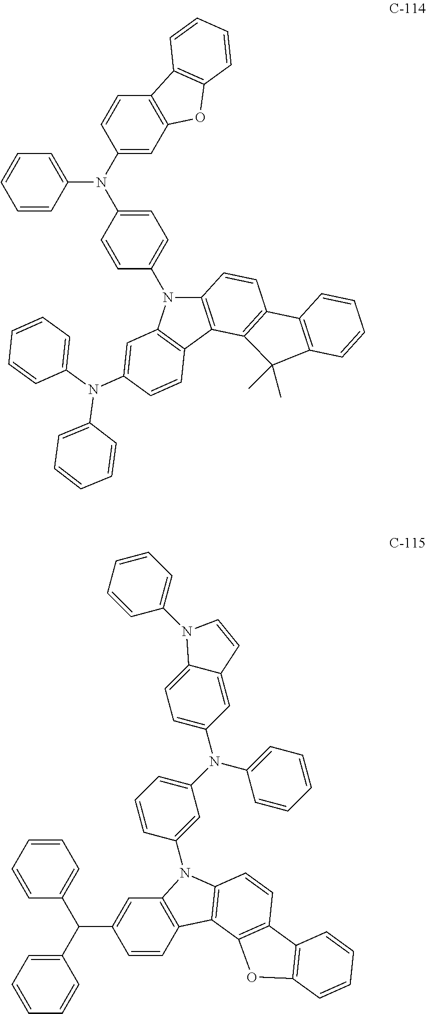 [SMILES: CC1(C)c2ccccc2-c2ccc3c(c21)c1ccc(N(c2ccccc2)c2ccccc2)cc1n3-c1ccc(N(c2ccccc2)c2ccc3c(c2)oc2ccccc23)cc1 ; c1ccc(C(c2ccccc2)c2ccc3c4c5oc6ccccc6c5ccc4n(-c4cccc(N(c5ccccc5)c5ccc6c(ccn6-c6ccccc6)c5)c4)c3c2)cc1]

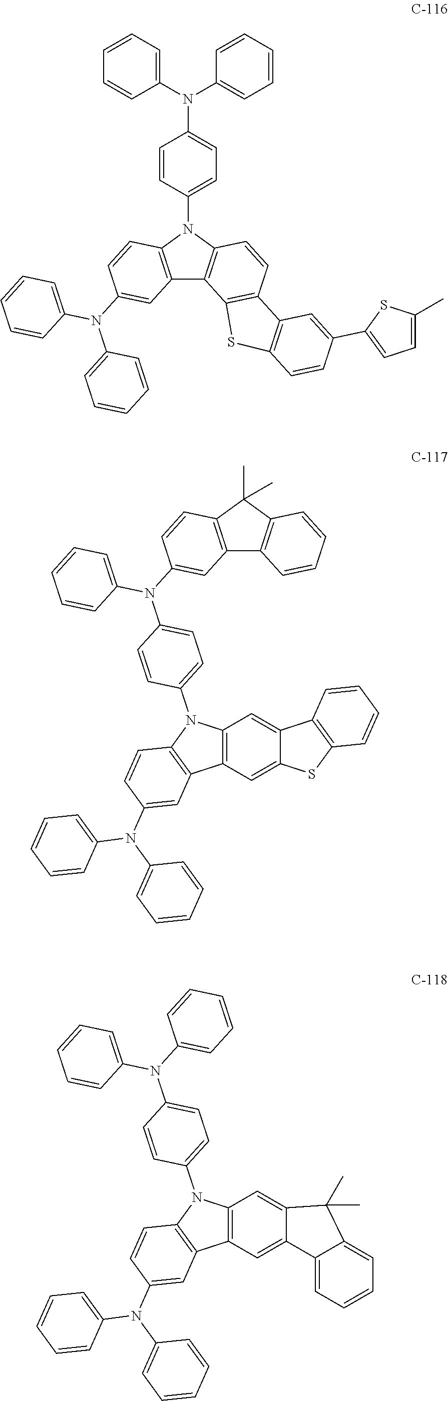 [SMILES: Cc1ccc(-c2ccc3sc4c(ccc5c4c4cc(N(c6ccccc6)c6ccccc6)ccc4n5-c4ccc(N(c5ccccc5)c5ccccc5)cc4)c3c2)s1 ; CC1(C)c2ccccc2-c2cc(N(c3ccccc3)c3ccc(-n4c5ccc(N(c6ccccc6)c6ccccc6)cc5c5cc6sc7ccccc7c6cc54)cc3)ccc21 ; CC1(C)c2ccccc2-c2cc3c4cc(N(c5ccccc5)c5ccccc5)ccc4n(-c4ccc(N(c5ccccc5)c5ccccc5)cc4)c3cc21]

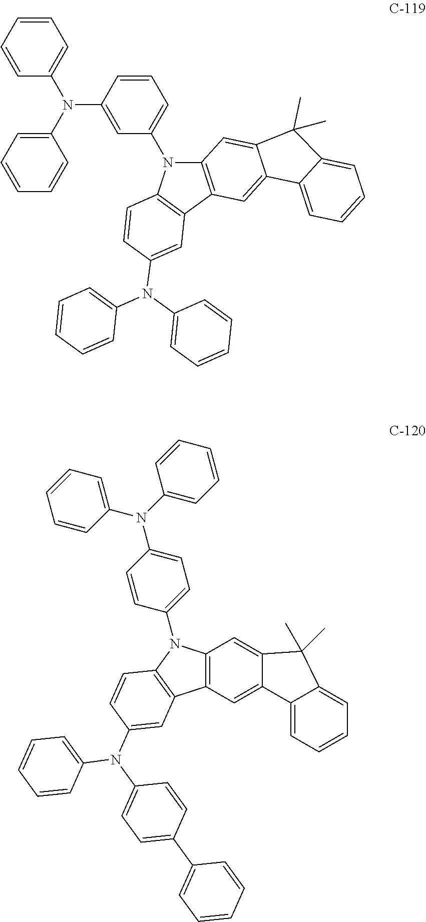 [SMILES: CC1(C)c2ccccc2-c2cc3c4cc(N(c5ccccc5)c5ccccc5)ccc4n(-c4cccc(N(c5ccccc5)c5ccccc5)c4)c3cc21 ; CC1(C)c2ccccc2-c2cc3c4cc(N(c5ccccc5)c5ccc(-c6ccccc6)cc5)ccc4n(-c4ccc(N(c5ccccc5)c5ccccc5)cc4)c3cc21]

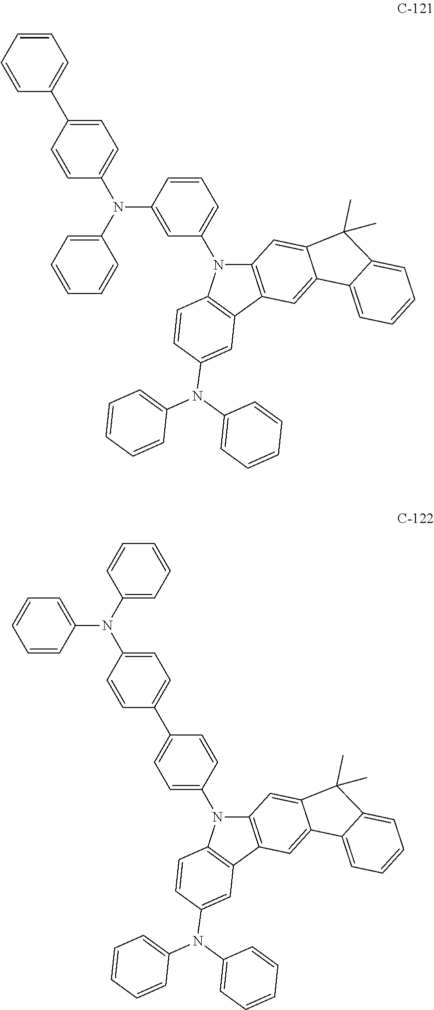 [SMILES: CC1(C)c2ccccc2-c2cc3c4cc(N(c5ccccc5)c5ccccc5)ccc4n(-c4cccc(N(c5ccccc5)c5ccc(-c6ccccc6)cc5)c4)c3cc21 ; CC1(C)c2ccccc2-c2cc3c4cc(N(c5ccccc5)c5ccccc5)ccc4n(-c4ccc(-c5ccc(N(c6ccccc6)c6ccccc6)cc5)cc4)c3cc21]

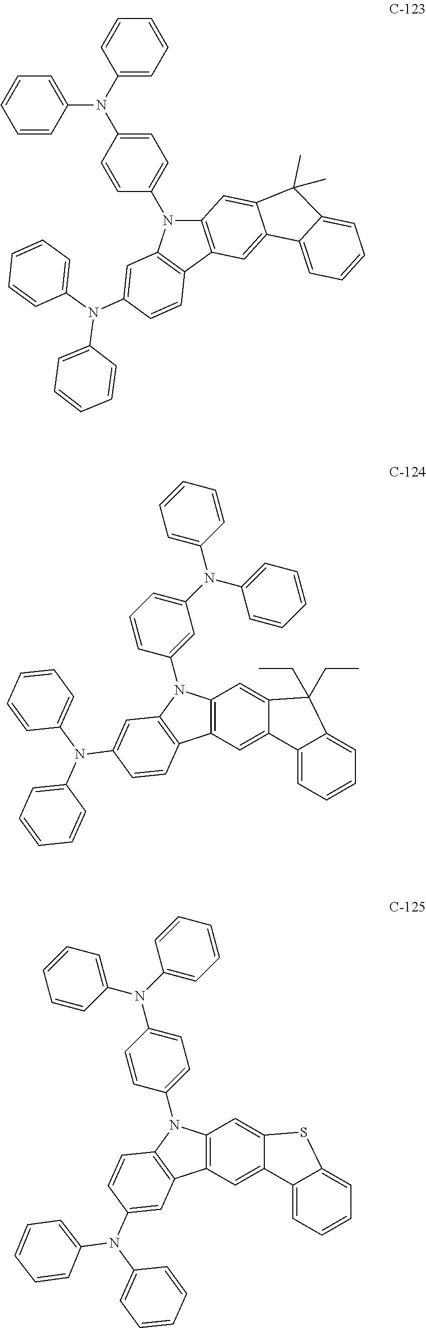 [SMILES: CC1(C)c2ccccc2-c2cc3c4ccc(N(c5ccccc5)c5ccccc5)cc4n(-c4ccc(N(c5ccccc5)c5ccccc5)cc4)c3cc21 ; CCC1(CC)c2ccccc2-c2cc3c4ccc(N(c5ccccc5)c5ccccc5)cc4n(-c4cccc(N(c5ccccc5)c5ccccc5)c4)c3cc21 ; c1ccc(N(c2ccccc2)c2ccc(-n3c4ccc(N(c5ccccc5)c5ccccc5)cc4c4cc5c(cc43)sc3ccccc35)cc2)cc1]

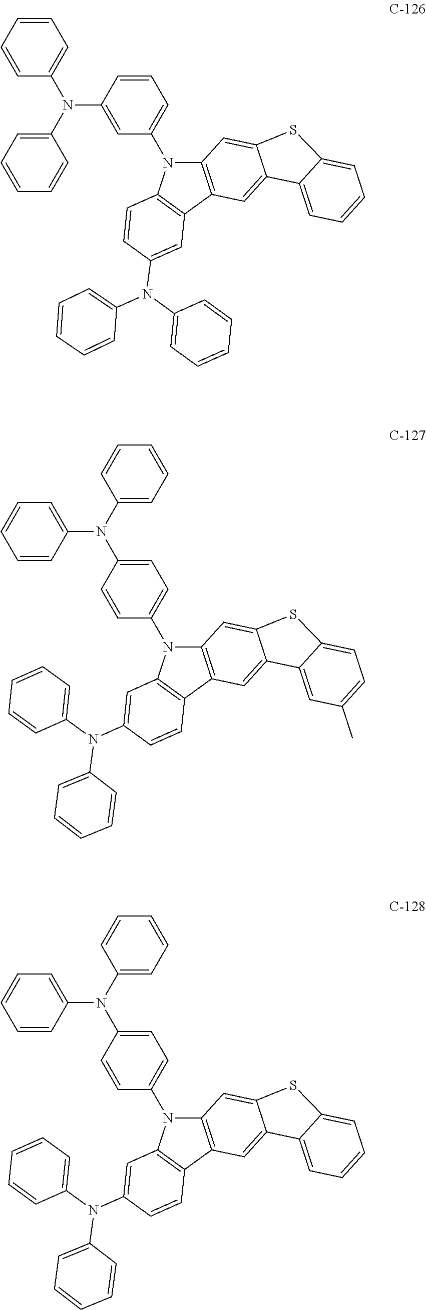 [SMILES: c1ccc(N(c2ccccc2)c2cccc(-n3c4ccc(N(c5ccccc5)c5ccccc5)cc4c4cc5c(cc43)sc3ccccc35)c2)cc1 ; Cc1ccc2sc3cc4c(cc3c2c1)c1ccc(N(c2ccccc2)c2ccccc2)cc1n4-c1ccc(N(c2ccccc2)c2ccccc2)cc1 ; c1ccc(N(c2ccccc2)c2ccc(-n3c4cc(N(c5ccccc5)c5ccccc5)ccc4c4cc5c(cc43)sc3ccccc35)cc2)cc1]

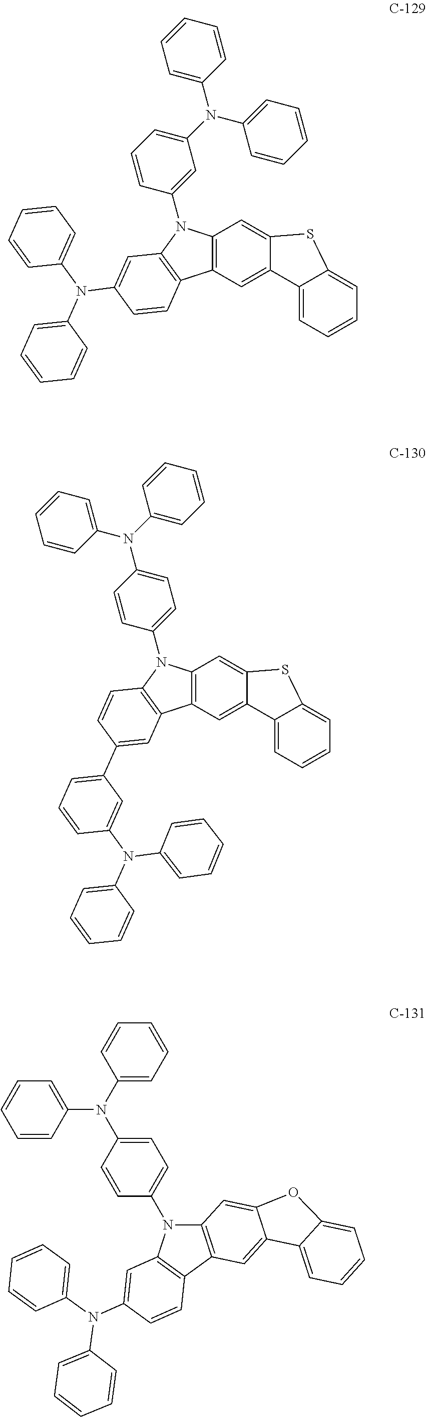 [SMILES: c1ccc(N(c2ccccc2)c2cccc(-n3c4cc(N(c5ccccc5)c5ccccc5)ccc4c4cc5c(cc43)sc3ccccc35)c2)cc1 ; c1ccc(N(c2ccccc2)c2ccc(-n3c4ccc(-c5cccc(N(c6ccccc6)c6ccccc6)c5)cc4c4cc5c(cc43)sc3ccccc35)cc2)cc1 ; c1ccc(N(c2ccccc2)c2ccc(-n3c4cc(N(c5ccccc5)c5ccccc5)ccc4c4cc5c(cc43)oc3ccccc35)cc2)cc1]

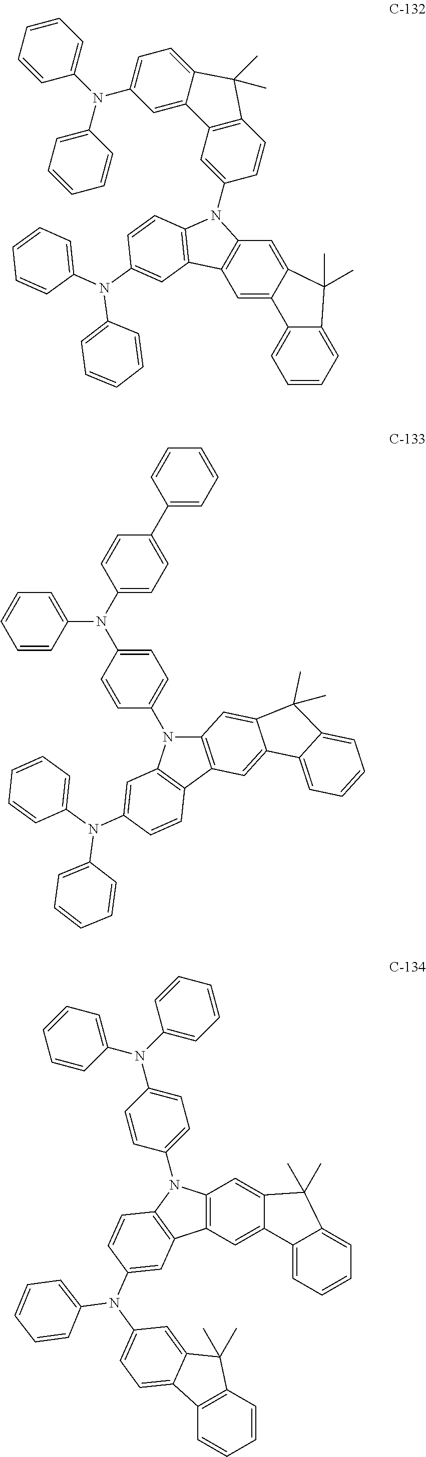 [SMILES: CC1(C)c2ccc(N(c3ccccc3)c3ccccc3)cc2-c2cc(-n3c4ccc(N(c5ccccc5)c5ccccc5)cc4c4cc5c(cc43)C(C)(C)c3ccccc3-5)ccc21 ; CC1(C)c2ccccc2-c2cc3c4ccc(N(c5ccccc5)c5ccccc5)cc4n(-c4ccc(N(c5ccccc5)c5ccc(-c6ccccc6)cc5)cc4)c3cc21 ; CC1(C)c2ccccc2-c2ccc(N(c3ccccc3)c3ccc4c(c3)c3cc5c(cc3n4-c3ccc(N(c4ccccc4)c4ccccc4)cc3)C(C)(C)c3ccccc3-5)cc21]

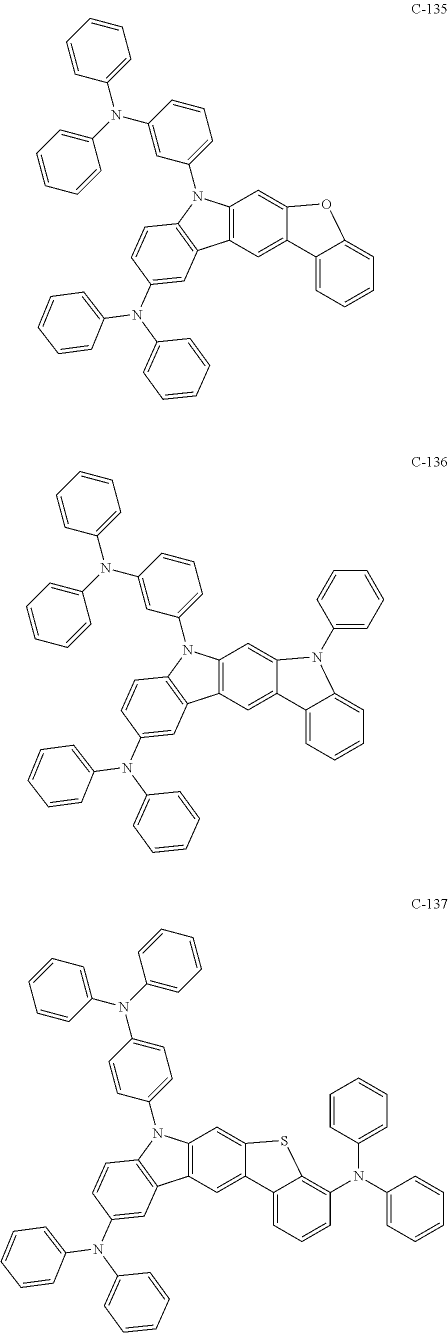 [SMILES: c1ccc(N(c2ccccc2)c2cccc(-n3c4ccc(N(c5ccccc5)c5ccccc5)cc4c4cc5c(cc43)oc3ccccc35)c2)cc1 ; c1ccc(N(c2ccccc2)c2cccc(-n3c4ccc(N(c5ccccc5)c5ccccc5)cc4c4cc5c6ccccc6n(-c6ccccc6)c5cc43)c2)cc1 ; c1ccc(N(c2ccccc2)c2ccc(-n3c4ccc(N(c5ccccc5)c5ccccc5)cc4c4cc5c(cc43)sc3c(N(c4ccccc4)c4ccccc4)cccc35)cc2)cc1]

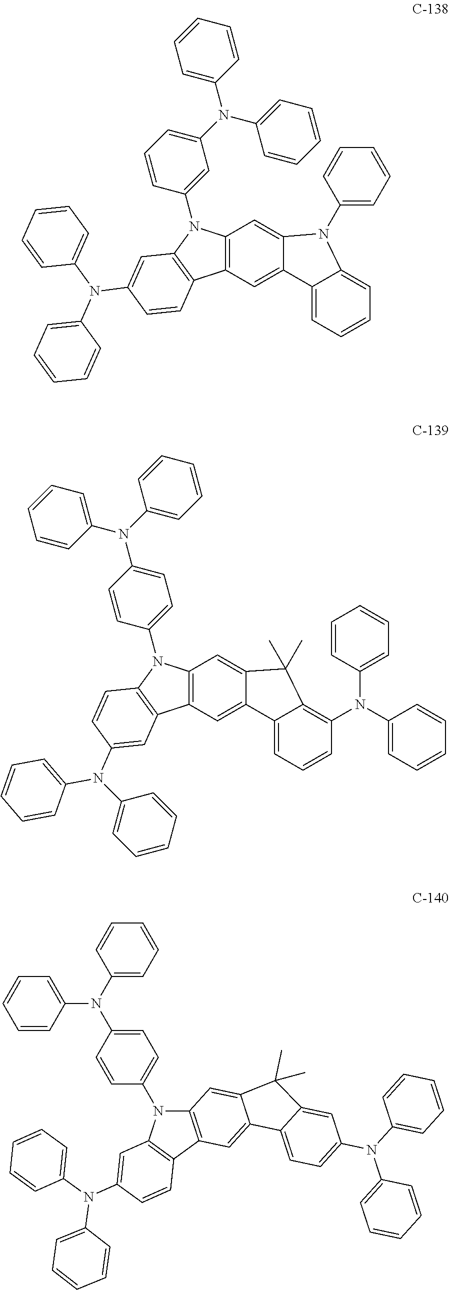 [SMILES: c1ccc(N(c2ccccc2)c2cccc(-n3c4cc(N(c5ccccc5)c5ccccc5)ccc4c4cc5c6ccccc6n(-c6ccccc6)c5cc43)c2)cc1 ; CC1(C)c2cc3c(cc2-c2cccc(N(c4ccccc4)c4ccccc4)c21)c1cc(N(c2ccccc2)c2ccccc2)ccc1n3-c1ccc(N(c2ccccc2)c2ccccc2)cc1 ; CC1(C)c2cc(N(c3ccccc3)c3ccccc3)ccc2-c2cc3c4ccc(N(c5ccccc5)c5ccccc5)cc4n(-c4ccc(N(c5ccccc5)c5ccccc5)cc4)c3cc21]

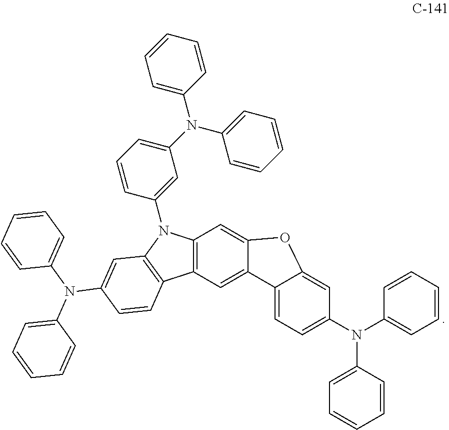 [SMILES: c1ccc(N(c2ccccc2)c2cccc(-n3c4cc(N(c5ccccc5)c5ccccc5)ccc4c4cc5c(cc43)oc3cc(N(c4ccccc4)c4ccccc4)ccc35)c2)cc1]

7. An organic electroluminescent device comprising the organic electroluminescent compound according to claim 1.
